(19) United States
(12) Patent Application Publication
(10) Pub. No.: US 2023/0204579 A1
(43) Pub. Date: Jun. 29, 2023

(54) Immune Cell Biomarkers of Cardiovascular Disease

(71) Applicant: La Jolla Institute for Immunology, La Jolla, CA (US)

(72) Inventor: Klaus Ley, La Jolla, CA (US)

(21) Appl. No.: 17/923,325

(22) PCT Filed: May 5, 2021
(86) PCT No.: PCT/US21/30946

§ 371 Date: Nov. 4, 2022

Related U.S. Application Data
(60) Provisional application No. 63/020,146, filed on May 5, 2020.

(51) Int. Cl. G01N 33/569; G01N 33/68

(52) U.S. Cl. CPC G01N 33/56972; G01N 33/6893; G01N 2333/705; G01N 2800/32; G01N 2800/50

(57) ABSTRACT

The present invention includes methods to determine a risk of a cardiovascular disease (CVD) or a subclinical CVD (sCVD) in a subject, by detecting a subset of intermediate monocyte cell populations in the immune cells, wherein a percentage of the subsets as compared to a total intermediate monocyte cell population present in the isolated immune cells is determined; comparing a proportion of the subsets subset of intermediate monocyte cell populations determined with a statistical sample representative of a proportion of equivalent subsets in a total intermediate monocyte cell populations from a subject that does not have a cardiovascular disease; and determining that the subject has an increased risk for cardiovascular disease where the subject has a decrease in the certain subsets, has an increase in another subset, or both, as compared to the statistical sample.

### CROSS-REFERENCE TO RELATED APPLICATIONS

This application claims priority to U.S. Provisional Application Ser. No. 63/020,146, filed May 5, 2020, the entire contents of which are incorporated herein by reference.

### STATEMENT OF FEDERALLY FUNDED RESEARCH

This invention was made with government support under grant/contract numbers HL 136275, HL 148094, and HL145241, awarded by the National Institutes of Health. The government has certain rights in this invention.

### TECHNICAL FIELD OF THE INVENTION

The present invention relates in general to the field of cardiovascular disease, and more particularly, to novel uses for immune cell populations and immune cell biomarkers of cardiovascular disease.

### BACKGROUND OF THE INVENTION

Without limiting the scope of the invention, its background is described in connection with an evaluation of risk of cardiovascular disease.

One such evaluation of risk for cardiovascular disease (CVD) is taught in U.S. Pat. No. 10,996,231, issued to Schaffer, et al., entitled, “Ceramides for evaluating risk of cardiovascular disease”, by detecting the presence of two very long chain ceramides, specifically ceramide 24:0, and optionally ceramide 22:0 from a biological sample by protein precipitation in a solution comprising isopropanol and chloroform, and determining the risk of cardiovascular disease.

Another such evaluation of risk for cardiovascular disease is taught in U.S. Pat. No. 10,996,229, issued to Wienhues-Thelen, et al., entitled “Use of IGFBP-7 in the assessment of heart failure”, which is said to disclose a method for assessing heart failure in vitro by measuring in a sample the concentration of the marker IGFBP-7, and of assessing heart failure by comparing the concentration determined in for IGFBP-7 compared to those established in a reference population. Also disclosed is the use of IGFBP-7 as a marker protein in the assessment of heart failure, a marker combination comprising IGFBP-7 and a kit for measuring IGFBP-7.

Despite these improvements, a need remains for improved methods of CVD or subclinical CVD detection, including the detection of the immune aspects of CVD or subclinical CVD.

### SUMMARY OF THE INVENTION

In one embodiment, the present invention includes a method to determine a risk of a cardiovascular disease (CVD) or a subclinical CVD (sCVD) in a subject, the method comprising: (a) obtaining a sample from the subject; (b) isolating immune cells from the sample; (c) detecting a first (INT1), second (INT2), third (INT3) and fourth (INT4) subset of intermediate monocyte cell populations in the immune cells, wherein a percentage of the INT1, INT2, INT3, and INT4 subsets as compared to a total intermediate monocyte cell population present in the isolated immune cells is determined; (d) comparing a proportion of the INT1, INT2, INT3, and INT4 subsets subset of intermediate monocyte cell populations determined in (c) with a statistical sample representative of a proportion of equivalent INT1, INT2, INT3, and INT4 subsets in a total intermediate monocyte cell populations from a subject that does not have a cardiovascular disease; and (e) determining that the subject has an increased risk for cardiovascular disease where the subject has a decrease in the INT2 and INT3 subsets, has an increase in the INT4 subset, or both, as compared to the statistical sample. In one aspect, if the subject is at increased risk based on the comparing step, then further treating the subject for cardiovascular disease. In another aspect, the intermediate monocyte cell population is detected in the isolated immune cells by gating for CD3−CD19−CD14+CD16+CD56− immune cells. In another aspect, the INT2 subset is detected by gating the isolated immune cells for IL6RA+, CD69+, CD86+, CD9+, CD141+, CD142+, CD163+, CCR2+, CCR4+, CCR5+, CD36+, CD38+, CD45RO+, CD206+. In another aspect, the INT3 subset is detected by gating the isolated immune cells for IL6RA, CD141+, CD142+, CD163+, CCR2+, CD36+, CD38+, CD69+, CD86+CD9+, CD154, CCR4+, CD69+, and optionally LAG3+, CD206+, CD137+, CCR4+, CCR5+, CD206, CD45RO+, CD4. In another aspect, the INT4 subset is detected by gating the isolated immune cells for IL6RA_{low}, CD137^{lower than INT 2 and 3}, CD154−, CD163^{low}, CCR2^{low}, CCR4^{low}, CCR5^{low}, CD206^{low}, LAG3−, CD36^{lower than INT2 and 3}, CD38^{low}, and optionally CD45RA, CD9+. In another aspect, the subject is determined to have a decrease in the INT2 subset as compared to the statistical sample, where the proportion of the INT2 subset is 40% of a total CD3−CD19−CD14+CD16+CD56− INT population in the statistical sample and less than 20% of a total CD3−CD19−CD14+CD16+CD56− INT population in the subject. In another aspect, the subject is determined to have a decrease in the INT2 subset as compared to the statistical sample where there is one half of an amount of INT2 cells present in the subject as compared to the statistical sample. In another aspect, the subject is determined to have a decrease in the INT3 subset as compared to the statistical sample, where the proportion of the INT3 subset is 30% of a total CD3−CD19−CD14+CD16+CD56− INT population in the statistical sample and 10% of the total CD3−CD19−CD14+CD16+CD56− INT population in the subject. In another aspect, the subject is determined to have a decrease in the INT3 subset as compared to the statistical sample where there is one-third of an amount of INT3 cells present in the subject as compared to the statistical sample. In another aspect, the subject is determined to have an increase in the INT4 subset as compared to the statistical sample, where the proportion of the INT4 subset is 12% of a total CD3−CD19−CD14+CD16+CD56− INT population in the statistical sample and 40% of a total CD3−CD19−CD14+CD16+CD56− INT population in the subject. In another aspect, the subject is determined to have an increase in the INT4 subset as compared to the statistical sample where there is roughly four times an amount INT4 cells present in the subject as compared to the statistical sample. In another aspect, the sample is a blood, serum, or plasma sample, and biomarkers are detected as a protein or a nucleic acid.

In another embodiment, the present invention includes a method for treating a cardiovascular disease (CVD) or a subclinical CVD (sCVD) in a subject, the method comprising: (a) obtaining a sample from the subject; (b) isolating immune cells from the sample; (c) detecting a first (INT1), second (INT2), third (INT3) and fourth (INT4) subset of intermediate monocyte cell populations in the immune cells, wherein a percentage of the INT1, INT2, INT3, and INT4 subsets as compared to a total intermediate monocyte cell population present in the isolated immune cells is determined; (d) comparing a proportion of the INT1, INT2, INT3, and INT4 subsets subset of intermediate monocyte cell populations determined in (c) with a statistical sample representative of the proportion of equivalent INT1, INT2, INT3, and INT4 subsets in the total intermediate monocyte cell populations from a subject that does not have a cardiovascular disease; (e) determining that the subject has a CVD or subclinical CVD, wherein if the subject has a decrease in the INT2 and INT3 subsets, has an increase in the INT4 subset, or both, as compared to the statistical sample, it is determined that the subject has a CVD or a subclinical CVD; and (g) where the subject has a CVD or a subclinical CVD based on the comparing step, then further providing the subject with a treatment that prevents or treats the cardiovascular disease. In another aspect, the intermediate monocyte cell population is detected in the isolated immune cells by gating for CD3−CD19−CD14+CD16+CD56− immune cells. In another aspect, the INT2 subset is detected by gating the isolated immune cells for IL6RA+, CD69+, CD86+, CD9+, CD141+, CD142+, CD163+, CCR2+, CCR4+, CCR5+, CD36+, CD38+, CD45RO+, CD206+. In another aspect, the INT3 subset is detected by gating the isolated immune cells for IL6RA, CD141+, CD142+, CD163+, CCR2+, CD36+, CD38+, CD69+, CD86+CD9+, CD154, CCR4+, CD69+, and optionally LAG3+, CD206+, CD137+, CCR4+, CCR5+, CD206, CD45RO+, CD4. In another aspect, the e INT4 subset is detected by gating the isolated immune cells for IL6RA_{low}, CD137^{lower than INT 2 and 3}, CD154−, CD163^{low}, CCR2^{low}, CCR4^{low}, CCR5^{low}, CD206^{low}, LAG3−, CD36^{lower than INT2 and 3}, CD38^{low}, and optionally CD45RA, CD9+. In another aspect, the subject is determined to have a decrease in the INT2 subset as compared to the statistical sample, where the proportion of the INT2 subset is 40% of a total CD3−CD19−CD14+CD16+CD56− INT population in the statistical sample and less than 20% of a total CD3−CD19−CD14+CD16+CD56− INT population in the subject. In another aspect, the subject is determined to have a decrease in the INT2 subset as compared to the statistical sample where there is one half of an amount of INT2 cells present in the subject as compared to the statistical sample. In another aspect, the subject is determined to have a decrease in the INT3 subset as compared to the statistical sample, where the proportion of the INT3 subset is 30% of a total CD3−CD19−CD14+CD16+CD56− INT population in the statistical sample and 10% of the total CD3−CD19−CD14+CD16+CD56− INT population in the subject. In another aspect, the subject is determined to have a decrease in the INT3 subset as compared to the statistical sample where there is one-third of an amount of INT3 cells present in the subject as compared to the statistical sample. In another aspect, the subject is determined to have an increase in the INT4 subset as compared to the statistical sample, where the proportion of the INT4 subset is 12% of a total CD3−CD19−CD14+CD16+CD56− INT population in the statistical sample and 40% of a total CD3−CD19−CD14+CD16+CD56− INT population in the subject. In another aspect, the subject is determined to have an increase in the INT4 subset as compared to the statistical sample where there is roughly four times an amount INT4 cells present in the subject as compared to the statistical sample. In another aspect, the sample is a blood, serum, or plasma sample, and biomarkers are detected as a protein or a nucleic acid.

In another embodiment, the present invention includes a method of diagnosing a cardiovascular disease (CVD) or a subclinical CVD (sCVD) in a subject, the method comprising: (a) obtaining a sample from the subject; (b) isolating immune cells from the sample; (c) detecting a first (INT1), second (INT2), third (INT3) and fourth (INT4) subset of intermediate monocyte cell populations in the immune cells, wherein a percentage of the INT1, INT2, INT3, and INT4 subsets as compared to a total intermediate monocyte cell population present in the isolated immune cells is determined; (d) comparing a proportion of the INT1, INT2, INT3, and INT4 subsets subset of intermediate monocyte cell populations determined in (c) with a statistical sample representative of the proportion of equivalent INT1, INT2, INT3, and INT4 subsets in the total intermediate monocyte cell populations from a subject that does not have a cardiovascular disease; and (e) determining that the subject has a cardiovascular disease or subclinical CVD, wherein if the subject has a decrease in the INT2 and INT3 subsets, has an increase in the INT4 subset, or both, as compared to the statistical sample, it is determined that the subject will develop or has a cardiovascular disease. In one aspect, the intermediate monocyte cell population is detected in the isolated immune cells by gating for CD3−CD19−CD14+CD16+CD56− immune cells. In another aspect, the INT2 subset is detected by gating the isolated immune cells for IL6RA+, CD69+, CD86+, CD9+, CD141+, CD142+, CD163+, CCR2+, CCR4+, CCR5+, CD36+, CD38+, CD45RO+, CD206+. In another aspect, the INT3 subset is detected by gating the isolated immune cells for IL6RA, CD141+, CD142+, CD163+, CCR2+, CD36+, CD38+, CD69+, CD86+CD9+, CD154, CCR4+, CD69+, and optionally LAG3+, CD206+, CD137+, CCR4+, CCR5+, CD206, CD45RO+, CD4. In another aspect, the INT4 subset is detected by gating the isolated immune cells for IL6RA_{low}, CD137^{lower than INT 2 and 3}, CD154−, CD163^{low}, CCR2^{low}, CCR4^{low}, CCR5^{low}, CD206^{low}, LAG3−, CD36^{lower than INT2 and 3}, CD38^{low}, and optionally CD45RA, CD9+. In another aspect, the subject is determined to have a decrease in the INT2 subset as compared to the statistical sample, where the proportion of the INT2 subset is 40% of a total CD3−CD19−CD14+CD16+CD56− INT population in the statistical sample and less than 20% of a total CD3−CD19−CD14+CD16+CD56− INT population in the subject. In another aspect, the subject is determined to have a decrease in the INT2 subset as compared to the statistical sample where there is one half of an amount of INT2 cells present in the subject as compared to the statistical sample. In another aspect, the subject is determined to have a decrease in the INT3 subset as compared to the statistical sample, where the proportion of the INT3 subset is 30% of a total CD3−CD19−CD14+CD16+CD56− INT population in the statistical sample and 10% of the total CD3−CD19−CD14+CD16+CD56− INT population in the subject. In another aspect, the subject is determined to have a decrease in the INT3 subset as compared to the statistical sample where there is one-third of an amount of INT3 cells present in the subject as compared to the statistical sample. In another aspect, the subject is determined to have an increase in the INT4 subset as compared to the statistical sample, where the proportion of the INT4 subset is 12% of a total CD3−CD19−CD14+CD16+CD56− INT population in the statistical sample and 40% of a total CD3−CD19−CD14+CD16+CD56− INT population in the subject. In another aspect, the subject is determined to have an increase in the INT4 subset as compared to the statistical sample where there is roughly four times an amount INT4 cells present in the subject as compared to the statistical sample. In another aspect, the sample is a blood, serum, or plasma sample, and biomarkers are detected as a protein or a nucleic acid.

In another embodiment, the present invention includes a method to determine whether a subject needs treatment for a cardiovascular disease (CVD) or a subclinical CVD (sCVD), the method comprising: (a) obtaining a sample from the subject; (b) isolating the immune cells from the sample; (c) measuring the presence of one or more immune cell subpopulations from the isolated immune cells, and an expression level of one or more biomarkers in the one or more immune cell subpopulations; (d) comparing the expression level of the one or more biomarkers in (c) with a statistical sample representative of the biomarkers present in an equivalent immune cell subpopulation of a subject not having a cardiovascular disease; and (e) determining that the subject is to be treated for the cardiovascular disease based on the comparing step of (d), wherein if the expression level of the one or more biomarkers present in the subject sample is higher than a baseline level of the statistical sample, the subject is determined to need treatment for the CVD or subclinical CVD. In another aspect, the method further comprises treating the subject in need of treatment for cardiovascular disease or subclinical CVD. In another aspect, the one or more immune cell subpopulations is selected from CD4 T cells or subpopulations thereof, CD8 T cells or subpopulations thereof, intermediate monocytes or subpopulations thereof, B cells or subpopulations thereof, and Natural Killer cells or subpopulations thereof. In another aspect, the sample is a blood, serum, or plasma sample, and the biomarkers are detected as a protein or a nucleic acid. In another aspect, the immune cell subpopulation consists essentially of T cells and the one or more biomarkers are selected from 1, 2, 3, 4, 5, 6, 7, 8, 9, 10, 15, 20, 25, 30, 40, or 50 biomarkers selected from: SELPLG; IFITM3; MYC; FYB; JUNB; IL32; SELL; CCR7; GIMAP5; TCF7; FAS; BCL6; TNFSF10; STAT3; CD27; BIRC3; IKZF1; PTPRC; CYTIP; IFITM2; CD2; LCK; IL7R; FOXO1; TRAC; CCA; CD69; ICOS; SLC2A3; LEF1; RGS1; IL4R; CD52; HOPX; GZMA; KLRG1; KLRD1; TIGIT; STAT1; KLRC1; LGALS1; KLRC4; CD63; SAMD3; S100A10; KLRK1; IL23R; CCL5; APOBEC3G; CD160; TARP; CD3D; CD8A; KLRD1; ITGA4; CX3CR1; VNN2; GZMA; CH3L2; or GIMAP5, and optionally the biomarkers are selected in the order listed. In another aspect, the immune cell subpopulation consists essentially of classical monocytes and the one or more biomarkers are selected from 1, 2, 3, 4, 5, 6, 7, 8, 9, 10, 15, 20, 25, 30, or 40 biomarkers selected from: CCL4; CCL3; SLC2A3; SOD2; SELPLG; CD14; LAP3; FYB; LYN; CD80; TNFSF10; TNFSF8; CSF3; CSF3R; CD36; NAIP; SELL; CLEC4E; IL8R; PTPRC; VCAN; ADGRE1; MNDA; DUSP1; FYB; DUSP2; LYZ; JUNB; SCREP1; DOCK8; CD300A; LAP3; TNFSF13; SDCP; MDX1; FYN; STAT6; IL1B; NAMPT; STAT3; IL6; IER3; TLR2; CD83; FYB; S100A9; TNFSF13B; CLEC4E; or ICAM1, and optionally the biomarkers are selected in the order listed. In another aspect, the immune cell subpopulation consists essentially of intermediate monocytes and the one or more biomarkers are selected from 1, 2, 3, 4, 5, 6, 7, 8, 9, or 10 biomarkers selected from: PRDM1; TNF; CCL4; LYZ; DUSP1; IL1B; DUSP2; CCL3; IER3; or ICAM1, and optionally the biomarkers are selected in the order listed. In another aspect, the immune cell subpopulation consists essentially of B cells and the one or more biomarkers are selected from 1, 2, 3, 4, 5, 6, 7, 8, or 9 biomarkers selected from: CD74; IGHD; CD79A; LYN; CD83; R3HDM4; CD79B; CXCR5; or SLC2A3, and optionally the biomarkers are selected in the order listed. In another aspect, the cardiovascular disease is selected from at least one of: coronary heart disease (CHD), heart failure (HF), coronary heart disease (CHD), cerebrovascular disease, peripheral arterial disease, atherosclerosis, or heart failure.

In another embodiment, the present invention includes a method to prevent cardiovascular disease (CVD) in a subject with unknown CVD status, the method comprising: (a) obtaining a sample from the subject; (b) isolating immune cells from the sample; (c) measuring a presence of one or more immune cell subpopulations from the isolated immune cells, and determining an expression level of one or more biomarkers in the one or more immune cell subpopulations; (d) comparing the expression level of the one or more biomarkers in (c) with a statistical sample representative of the biomarkers in an equivalent immune cell subpopulation of a subject not having a cardiovascular disease; (e) determining the CVD status of the subject based on the comparing step of (d), wherein if the expression level of the one or more biomarkers present in the subject sample is higher than a baseline level of the statistical sample, the subject is determined to need treatment to prevent CVD; and (f) administering a treatment to prevent CVD in the subject. In one aspect, the treatment to prevent CVD comprises administering a statin. In another aspect, the one or more immune cell subpopulation is selected from CD4 T cells or subpopulations thereof, CD8 T cells or subpopulations thereof, intermediate monocytes or subpopulations thereof, B cells or subpopulations thereof, and Natural Killer cells or subpopulations thereof. In another aspect, the sample is a blood, serum, or plasma sample, and the biomarkers are detected as a protein or a nucleic acid. In another aspect, the immune cell subpopulation consists essentially of T cells and the one or more biomarkers are selected from 1, 2, 3, 4, 5, 6, 7, 8, 9, 10, 15, 20, 25, 30, 40, or 50 biomarkers selected from: SELPLG; IFITM3; MYC; FYB; JUNB; IL32; SELL; CCR7; GIMAP5; TCF7; FAS; BCL6; TNFSF10; STAT3; CD27; BIRC3; IKZF1; PTPRC; CYTIP; IFITM2; CD2; LCK; IL7R; FOXO1; TRAC; CCA; CD69; ICOS; SLC2A3; LEF1; RGS1; IL4R; CD52; HOPX; GZMA; KLRG1; KLRD1; TIGIT; STAT1; KLRC1; LGALS1; KLRC4; CD63; SAMD3; S100A10; KLRK1; IL23R; CCL5; APOBEC3G; CD160; TARP; CD3D; CD8A; KLRD1; ITGA4; CX3CR1; VNN2; GZMA; CH3L2; or GIMAP5, and optionally the biomarkers are selected in the order listed. In another aspect, the immune cell subpopulation consists essentially of classical monocytes and the one or more biomarkers are selected from 1, 2, 3, 4, 5, 6, 7, 8, 9, 10, 15, 20, 25, 30, or 40 biomarkers selected from: CCL4; CCL3; SLC2A3; SOD2; SELPLG; CD14; LAP3; FYB; LYN; CD80; TNFSF10; TNFSF8; CSF3; CSF3R CD36; NAIP; SELL; CLEC4E; IL8R; PTPRC; VCAN; ADGRE1; MNDA; DUSP1; FYB; DUSP2; LYZ; JUNB; SCREP1; DOCK8; CD300A; LAP3; TNFSF13; SDCP; MDX1; FYN; STAT6; IL1B; NAMPT; STAT3; IL6; IER3; TLR2; CD83; FYB; S100A9; TNFSF13B; CLEC4E; or ICAM1, and optionally the biomarkers are selected in the order listed. In another aspect, the immune cell subpopulation consists essentially of intermediate monocytes and the one or more biomarkers are selected from 1, 2, 3, 4, 5, 6, 7, 8, 9, or 10 biomarkers selected from: PRDM1; TNF; CCL4; LYZ; DUSP1; IL1B; DUSP2; CCL3; IER3; or ICAM1, and optionally the biomarkers are selected in the order listed. In another aspect, the immune cell subpopulation consists essentially of B cells and the one or more biomarkers are selected from 1, 2, 3, 4, 5, 6, 7, 8, or 9 biomarkers selected from: CD74; IGHD; CD79A; LYN; CD83; R3HDM4; CD79B; CXCR5; or SLC2A3, and optionally the biomarkers are selected in the order listed. In another aspect, the cardiovascular disease is selected from at least one of: coronary heart disease (CHD), heart failure (HF), coronary heart disease (CHD), cerebrovascular disease, peripheral arterial disease, atherosclerosis, or heart failure.

In another embodiment, the present invention includes a method of treating a subject that has or will develop a cardiovascular disease, the method comprising: (a) obtaining a sample from the subject; (b) isolating immune cells from the sample; (c) measuring a presence of one or more immune cell subpopulations from the isolated immune cells, and an expression level of one or more biomarkers in the one or more immune cell subpopulations; (d) comparing the expression level of the one or more biomarkers in (c) with a statistical sample representative of the biomarkers in an equivalent immune cell subpopulation of a subject not having a cardiovascular disease; and (e) ruling-out the subject as having cardiovascular disease from a diagnostic test for cardiovascular disease, a treatment of cardiovascular disease, or a combination thereof, based on the comparing step of (d), wherein if the expression level of the one or more biomarkers present in the subject sample is lower than a baseline level of the statistical sample, the subject is determined not to have or be likely to develop CVD; or (f) administering a preventive treatment for the cardiovascular disease, a treatment for cardiovascular disease, or a combination thereof, to the subject who is at risk of developing the cardiovascular disease based on the comparing step of (d), wherein if the expression level of the one or more biomarkers present in the subject sample is higher than a baseline level of the statistical sample, the subject is to have or be likely to develop CVD. In one aspect, the one or more immune cell subpopulations is selected from CD4 T cells or subpopulations thereof, CD8 T cells or subpopulations thereof, intermediate monocytes or subpopulations thereof, B cells or subpopulations thereof, and Natural Killer cells or subpopulations thereof. In another aspect, the sample is a blood, serum, or plasma sample, and the biomarkers are detected as a protein or a nucleic acid. In another aspect, the immune cell subpopulation consists essentially of T cells and the one or more biomarkers are selected from 1, 2, 3, 4, 5, 6, 7, 8, 9, 10, 15, 20, 25, 30, 40, or 50 biomarkers selected from: SELPLG; IFITM3; MYC; FYB; JUNB; IL32; SELL; CCR7; GIMAP5; TCF7; FAS; BCL6; TNFSF10; STAT3; CD27; BIRC3; IKZF1; PTPRC; CYTIP; IFITM2; CD2; LCK; IL7R; FOXO1; TRAC; CCA; CD69; ICOS; SLC2A3; LEF1; RGS1; IL4R; CD52; HOPX; GZMA; KLRG1; KLRD1; TIGIT; STAT1; KLRC1; LGALS1; KLRC4; CD63; SAMD3; S100A10; KLRK1; IL23R; CCL5; APOBEC3G; CD160; TARP; CD3D; CD8A; KLRD1; ITGA4; CX3CR1; VNN2; GZMA; CH3L2; or GIMAP5, and optionally the biomarkers are selected in the order listed. In another aspect, the immune cell subpopulation consists essentially of classical monocytes and the one or more biomarkers are selected from 1, 2, 3, 4, 5, 6, 7, 8, 9, 10, 15, 20, 25, 30, or 40 biomarkers selected from: CCL4; CCL3; SLC2A3; SOD2; SELPLG; CD14; LAP3; FYB; LYN; CD80; TNFSF10; TNFSF8; CSF3; CSF3R; CD36; NAIP; SELL; CLEC4E; IL8R; PTPRC; VCAN; ADGRE1; MNDA; DUSP1; FYB; DUSP2; LYZ; JUNB; SCREP1; DOCK8; CD300A; LAP3; TNFSF13; SDCP; MDX1; FYN; STAT6; IL1B; NAMPT; STAT3; IL6; IER3; TLR2; CD83; FYB; S100A9; TNFSF13B; CLEC4E; or ICAM1, and optionally the biomarkers are selected in the order listed. In another aspect, the immune cell subpopulation consists essentially of intermediate monocytes and the one or more biomarkers are selected from 1, 2, 3, 4, 5, 6, 7, 8, 9, or 10 biomarkers selected from: PRDM1; TNF; CCL4; LYZ; DUSP1; IL1B; DUSP2; CCL3; IER3; or ICAM1, and optionally the biomarkers are selected in the order listed. In another aspect, the immune cell subpopulation consists essentially of B cells and the one or more biomarkers are selected from 1, 2, 3, 4, 5, 6, 7, 8, or 9 biomarkers selected from: CD74; IGHD; CD79A; LYN; CD83; R3HDM4; CD79B; CXCR5; or SLC2A3, and optionally the biomarkers are selected in the order listed. In another aspect, the cardiovascular disease is selected from at least one of: coronary heart disease (CHD), heart failure (HF), coronary heart disease (CHD), cerebrovascular disease, peripheral arterial disease, atherosclerosis, or heart failure. In another aspect, the method further comprises referring the subject to a specialist in cardiovascular disease. In another aspect, in the various methods, step (b) is optionally omitted, and the immune cell subpopulations are measured directly from the sample.

### BRIEF DESCRIPTION OF THE DRAWINGS

For a more complete understanding of the features and advantages of the present invention, reference is now made to the detailed description of the invention along with the accompanying figures and in which:

FIG. 1A to IE. Gating scheme (FIG. 1A) and biaxial dot plots (FIGS. 1B to IE) to identify major cell types. PBMCs from 32 WIHS participants were hash-tagged and stained with 40 oligonucleotide-tagged mAbs. (FIG. 1B) B cells were defined as CD19+CD3− and T cells as CD19−CD3+. (FIG. 1C) T cells were identified as CD4 (CD4+CD8−) or CD8 (CD4−CD8+). (FIG. 1D) Monocytes: All CD19−CD3− cells were gated for CD14 and CD16, with CD14+CD16− cells being classical (CM) and CD14+CD16+ being intermediate (INT) monocytes. (FIG. 1E) NK cells: The CD14−CD16+ cells from panel D were gated for CD56 to identify NK cells. Remaining CD56−CD16+ were nonclassical monocytes (NCM).

FIGS. 2A to 2G show antibody-based UMAP clustering of major cell types. The possible cell type was defined by surface marker expression pattern. (FIG. 2A) CD4 T cells: Cluster 1 is effector memory (EM) (CD45RA−CCR7−). Clusters 2 and 4 are naïve (CD45RA+CCR7+, labeled Naive1 and Naive2, respectively). Cluster 3 is Th1 EM (CD45RA−CCR7−CXCR3+CCR4−CCR6−). Cluster 5 is Th2 EM (CD45RA−CCR7−CXCR3−CCR4+CCR6−). Cluster 6 is follicular helper EM T cell (Tfh EM) (CD45RA-CCR7−CXCR5+). Cluster 7 is NKT terminally differentiated EM (NKT EMRA) (CD45RA+CD56+CCR7−). Cluster 8 is EM with some CCR2 and CCR5 expression (CCR2+CCR5+EM). Cluster 9 is Treg (CD25hiCD127low). Cluster 10 is Th17 EM (CD45RA−CCR7−CXCR3−CCR4−CCR6+). Cluster 11 is CXCR3+CXCR5+EM (CD45RA−CCR7−CXCR3+CXCR5+CCR4−CCR6−). (FIG. 2B) CD8 T cells: Clusters 1, 5, 8, 11, 12, 13 and 14 are EMRA (CD45RA+CCR7−). Cluster 1 is labeled EMRA1. Cluster 5 is NKT EMRA (labeled NKT EMRA1) (CD45RA+CCR7−CD56+). Cluster 8 is CD11c+NKT EMRA (labeled NKT EMRA2). Cluster 11 is CD20+EMRA. Cluster 12 is CXCR3+EMRA. Cluster 13 is labeled EMRA2. Cluster 14 is CD16+. Cluster 2 is CD27hi naïve (CD45RA+CCR7+). Cluster 3, 7 and 10 are EM (CD45RA−CCR7−). Cluster 3 is CCR5+EM with CD27 expression. Cluster 7 is CD25+CXCR3+EM. Cluster 10 is CXCR3+CXCR5+EM. Cluster 4 is CD27+[labeled CD8 T (C4)]. Cluster 6 is CD69+NKT (CD9+CD36+CD56+CD69+). Cluster 9 is CCR4+CCR5+CCR6+. (FIG. 2C) Classical monocytes (CM): Cluster 2 is CD9−CD11c+CCR2+. Cluster 3 is HLA-DRlow. Cluster 4 is CD9highCD36high with CXCR3 expression. Cluster 5 is CD45RA+. Cluster 6 is myeloid dendritic cell (DC) (CD2hiCD11blowCD11c+CD206+HLA−DR+). There is a gradient for CD9, CD11c, CD36, CD69, CD86, CD137, CD142, CD152, CD163, CXCR4, CCR4, CCR6, and CCR7, left (cluster 4 and 5) to right. Cluster 1 is labeled as CM (C1) (FIG. 2D) Intermediate monocytes (INT): All clusters are CD86hi. They have a gradient, highest at bottom right (cluster 4), for CD9, CD36, CD69, CD137, CD142, CXCR4, CCR4, and CCR6. Gradient highest at bottom left (cluster 1): CD11c, CD127, CD152 and CXCR3. Cluster 4 is CD9+CD36+CCR2+. The others are labeled INT (C1), (C2) and (C3), respectively. (FIG. 2E) Nonclassical monocytes (NCM): CD2 gradient across all clusters, highest in bottom left (cluster 2). CD127, CD152 and CXCR3 has a gradient across all clusters, highest at bottom left and top right, low in the middle. Cluster 1 is CD9−CD16highCD36−. Cluster 4 is CD9+CD36+ with CD69 and CXCR4 expressions. The others are labeled as NCM (C2) and (C3), respectively. (FIG. 2F) B cells: Cluster 1 is pre-plasmablast (CD38dim) with CXCR4 and CCR6 expressions. Cluster 2 is resting memory (CD27+CXCR3+). Cluster 3 is naïve. Cluster 4 is plasmablast (CD38high). Cluster 5 is CD11c+ memory with high CD20 expression. Cluster 6 is activated memory with CD25 expression. Cluster 7 is a mix of myeloid markers. (FIG. 2G) NK cells: Cluster 1, 2 and 3 are mature NK cell (CD16+CD56+). Cluster 1 is labeled mature1 (CD2+CD152+), Cluster 2 is labeled mature2 (CD2-CD38+). Cluster 3 is labeled mature3 (CD2+CD11b+CD38+CD152+). Cluster 4 is immature NK cell (CD16−CD56bright) with CD25, CD27, CD137 and LAG3 expressions.

FIGS. 3A to 3F show significantly differential gene expression of cells in each cluster, compared to all other clusters of the parent cell type. Expression of 485 transcripts was determined by targeted amplification (BD Rhapsody system). Significant genes have adjusted p<0.05 and log 2 fold change >1. Dot plot: fraction of cells in cluster expressing each gene shown by size of circle and level of expression shown from white (=0) to dark (=max, log 2 scale). Bars indicate genes that were significantly differentially expressed in each cluster. (FIG. 3A) CD4 T cells, (FIG. 3B) CD8 T cells, (FIG. 3C) monocytes (Classical monocytes; CM, Intermediate monocytes; INT, Nonclassical monocytes; NCM), (FIG. 3D) B cells. Cluster description is as in FIG. 2 and table 2. Abbreviation: EM; effector memory, Tfh; follicular helper T, EMRA; terminally differentiated EM.

FIGS. 4A to 4H show significant correlations between all genes and all surface markers. All correlations between all genes and all surface markers were calculated and filtered for those that were statistically (Spearman's correlation test) significant and whose |r|>0.25 for the individual cell types (0.5 for all cells pooled). The r value of each correlation was color-coded from blue (=mostly negative correlation) to red (mostly positive correlation, as per color bar in each panel). (FIG. 4A) All cells, (FIG. 4B) CD4 T cells, (FIG. 4C) CD8 T cells, (FIG. 4D) Classical monocytes, (FIG. 4E) Intermediate monocytes, (FIG. 4F) Nonclassical monocytes, (FIG. 4G) B cells, (FIG. 4H) NK cells.

FIGS. 5A to 5Q show, Volcano plots comparing gene expression in single cells from WIHS participant types in each cluster. All 3 meaningful comparisons were calculated, but this figure is focused on HIV+CVD− vs HIV+CVD+, and HIV+CVD+ vs HIV+CVD+ with cholesterol medication; all clusters in which at least 10 genes were statistically significant. Colored dots (HIV+CVD− (left), HIV+CVD+ (center panels), and HIV+CVD+ with cholesterol medication (right) indicate 1 significantly differentiated expressed genes (FDR<0.05 and |log 2FC|>2). 3 CD4 T and 7 CD8 T cell clusters, 5 CM and 1 each INT and B cell clusters met these criteria. Dashed line indicates adjusted p-value of 0.05.

FIG. 6 shows cell proportions in women with 1) HIV, 2) HIV and CVD, 3) HIV and CVD treated with cholesterol-lowering drugs and 4) women without HIV or CVD (healthy). 8 samples per group. Proportions of cells in each cluster calculated as percent of the parent cell type as indicated in the title of each panel. Only clusters with significant differences (*, p<0.05, **, P<0.01, *** p<0.001, **** p<0.0001) in cell proportions between (by log odds ratio) are shown. Abbreviation: EM; effector memory, EMRA; terminally differentiated EM.

### DETAILED DESCRIPTION OF THE INVENTION

While the making and using of various embodiments of the present invention are discussed in detail below, it should be appreciated that the present invention provides many applicable inventive concepts that can be embodied in a wide variety of specific contexts. The specific embodiments discussed herein are merely illustrative of specific ways to make and use the invention and do not delimit the scope of the invention.

To facilitate the understanding of this invention, a number of terms are defined below. Terms defined herein have meanings as commonly understood by a person of ordinary skill in the areas relevant to the present invention. Terms such as “a”, “an” and “the” are not intended to refer to only a singular entity, but include the general class of which a specific example may be used for illustration. The terminology herein is used to describe specific embodiments of the invention, but their usage does not delimit the invention, except as outlined in the claims.

The present disclosure describes experimental results and line of reasoning supports the development of a diagnosis assay/methods of diagnosing, and/or treatment approach, for cardiovascular disease, including subclinical cardiovascular disease, more so than what has been previously described.

As used herein, a “blood sample” refers to a biological sample derived from blood, preferably peripheral (or circulating) blood, e.g., whole blood. In certain embodiments, whole blood is preferred as the source for the lymphocytes used to detect the biomarkers as the samples are readily available and often obtained for other sampling, is stable, and requires less processing, thus making it ideal for locations with little to refrigeration or electricity, is easily transportable, and is commonly handled by medical support staff.

As used herein, the terms “normal,” “control,” and “healthy,” refer generally to a subject or individual who does not have, is not, and/or has not been diagnosed with, and/or is asymptomatic for a particular disease or disorder, specifically, a cardiovascular disorder, or one or more symptoms of cardiovascular disease. As such, a normal subject is a subject that does not have a cardiovascular disease. The terms can also refer to a sample obtained from such subject or individual. The cardiovascular disease or disorder under analysis or comparison is determinative of whether the subject is a “control” in that situation and for that cardiovascular disease or disorder. For example, where the level of a particular biomarker is obtained from an individual known to have cardiovascular disease, that subject can be the “cardiovascular disease subject.” The level of the marker thus obtained from the cardiovascular disease subject can be compared to a level of that same biomarker from a subject who is not diagnosed with cardiovascular disease and who is known not to have prevalent cardiovascular disease, i.e., a “normal subject.” Thus, “normal subject” in this example refers to a subject that does not have a cardiovascular disorder or cardiovascular disease. A “normal” individual or a sample from a “normal” individual can also refer to quantitative data, qualitative data, or both from an individual who has or would be assessed by a physician as not having a disease, e.g., a cardiovascular disease. Often, a “normal” individual is also age-matched within a range of 1, 2, 3, 4, 5, 6, 7, 8, 9 or 10 years with the sample of the individual to be assessed.

As used herein, the term “treatment” refers to the alleviation, amelioration, and/or stabilization of symptoms, as well as delay in progression of symptoms of a particular disorder. For example, “treatment” of cardiovascular disease includes any one or more of: (1) elimination of one or more symptoms or biochemical markers of cardiovascular disease, (2) reduction of one or more symptoms or biochemical markers of cardiovascular disease, (4) stabilization of the symptoms or biochemical markers of cardiovascular disease (e.g., failure to progress to more advanced stages of cardiovascular disease), and (5) delay in onset of one or more symptoms or biochemical markers of cardiovascular disease delay in progression (i.e., worsening) of one or more symptoms of cardiovascular disease; and (6) delay in progression (i.e., worsening) of one or more symptoms or biochemical markers of cardiovascular disease.

As used herein, a “statistical sample representative of the subject” or a “statistical sample representative of the patient” refers to a statistical sample comprising one or more of the following groups of individuals: (1) individuals with a family history of cardiovascular disease; (2) individuals with no family history of cardiovascular disease; (3) individuals who have cardiovascular disease; and (4) individuals who do not have cardiovascular disease.

As used herein, an “effective amount” refers to a monoamine oxidase inhibitor, an antioxidant, or both, in an amount necessary or sufficient to treat a subject, or to provide improvement, benefit, or enhancement to health or performance. An improvement, benefit, or enhancement to health or performance can include, e.g., preventing or reducing cardiovascular disease symptoms related to cardiovascular disease. It can also include increase the length of time in which a subject does not need additional treatment for symptoms of cardiovascular disease such as treatment with a statin. The effective amount can vary depending on such factors as the size and weight of the subject, the type of result or outcome, or the particular quercetin compound. For example, the choice of a statin can affect what constitutes an “effective amount”. One of ordinary skill in the art would be able to study the aforementioned factors and make the determination regarding the effective amount of the quercetin compound without undue experimentation.

As used herein, aa “predictive biomarker” refers to a biomarker used to identify individuals who are more likely than similar patients without the biomarker to experience a favorable or unfavorable effect from a specific intervention or exposure.

Cardiovascular disease (CVD) is the leading cause of death worldwide, but it is subclinical and remains undetected for a long time, often years. Current biomarkers of CVD are LDL cholesterol, blood pressure, BNP and galectin-3. They explain some of the CVD risk, but are not sufficient. To assess CVD risk, people undergo expensive MRI, CT or ultrasound scans. Commonly used tests are coronary calcium scores (CAC), carotid ultrasound (IMT or plaque count) or coronary angiography scores (SYNTAX or Gensini). Inflammatory CVD risk can be measured by CRP or IL-6; they are not widely used. The immune risk for atherosclerosis is well documented, but is not currently measurable. The estimated immune risk for CVD is 30-50% of the total risk. Here, the discovery of immune cells in blood that indicate subclinical CVD (carotid plaque count) is disclosed. These cells are easily measurable by flow cytometry. As such, embodiments of the present invention are drawn to a method of diagnosing CVD in a subject, the method comprising testing a subject's sample for one or more of the biomarkers listed herein.

As embodied and broadly described herein, the present disclosure relates to method to determine a risk of a cardiovascular disease (CVD) or a subclinical CVD (sCVD) in a subject, method for treating a cardiovascular disease (CVD) or a subclinical CVD (sCVD) in a subject, method of diagnosing CVD a cardiovascular disease (CVD) or a subclinical CVD (sCVD) in a subject, method of reducing or eliminating an immune response against CVD or subclinical CVD in a subject, in which the methods comprise contacting cells of the subject with an effective amount of an agent capable of treating or preventing cardiovascular disease. In certain embodiments, an immune response against CVD or subclinical CVD is induced, enhanced, or sustained in the subject subsequent to detection in the subject the presence or absence of intermediate monocyte cell populations. The method generally includes the steps of: (a) obtaining a sample from the subject; (b) isolating immune cells from the sample; (c) detecting a first (INT1), second (INT2), third (INT3) and fourth (INT4) subset of intermediate monocyte cell populations in the immune cells, wherein a percentage of the INT1, INT2, INT3, and INT4 subsets as compared to a total intermediate monocyte cell population present in the isolated immune cells is determined; (d) comparing a proportion of the INT1, INT2, INT3, and INT4 subsets subset of intermediate monocyte cell populations determined in (c) with a statistical sample representative of the proportion of equivalent INT1, INT2, INT3, and INT4 subsets in the total intermediate monocyte cell populations from a subject that does not have a cardiovascular disease; and (e) determining that the subject has an increased risk for cardiovascular disease where the subject has a decrease in the INT2 and INT3 subsets, has an increase in the INT4 subset, or both, as compared to the statistical sample. In one embodiment, if the subject is at increased risk based on the comparing step, then further treating the subject for cardiovascular disease. Examples of intermediate monocyte cell population detected from the isolated immune cells are those gated for CD3−CD19−CD14+CD16+CD56− immune cells.

In certain non-limiting examples, the proportion of the INT2 subset decreases by 5, 10, 15, 20, 30, 40, 50, 60, 70, 80, 90, or 100% from the normal levels of INT2, that is, a subject or population of subjects that does not have cardiovascular disease (CVD) or subclinical CVD (sCVD). Alternatively, the fold-decrease of INT2 cells can be 1.1, 1.2, 1.3, 1.4, 1.5, 2, 5, 10, 20, 25, 50, 75 or 100-fold. In one example, the INT2 is about 40% of a total CD3−CD19−CD14+CD16+CD56− INT population in the normal statistical sample, and less than 20, 15, 10, or 5% of a total CD3−CD19−CD14+CD16+CD56− INT population in the subject. The subject is determined to have a decrease in the INT2 subset as compared to the statistical sample where there is one-half, one-third, one-fourth, one-fifth, or less of the amount of INT2 cells present in the subject as compared to the statistical sample.

In certain non-limiting examples, the proportion of the INT3 subset decreases by 5, 10, 15, 20, 30, 40, 50, 60, 70, 80, 90, or 100% from the normal levels of INT3, that is, a subject or population of subjects that does not have cardiovascular disease (CVD) or subclinical CVD (sCVD). The subject is determined to have a decrease in the INT3 subset as compared to the statistical sample, where the proportion of the INT3 subset is 30% of a total CD3−CD19−CD14+CD16+CD56− INT population in the normal statistical sample, and 10, 9, 8, 7, 6, 5, 4, 3, 2, or 1% of the total CD3−CD19−CD14+CD16+CD56− INT population in the subject. Alternatively, the fold-decrease of INT3 cells can be 1.1, 1.2, 1.3, 1.4, 1.5, 2, 5, 10, 20, 25, 50, 75 or 100-fold. The subject is determined to have a decrease in the INT3 subset as compared to the statistical sample where there is one-third, one-fourth, one-fifth, or less of the amount of INT3 cells present in the subject as compared to the statistical sample.

In certain non-limiting examples, the proportion of the INT4 subset increases by 5, 10, 15, 20, 30, 40, 50, 60, 70, 80, 90, or 100% from the normal levels of INT4, that is, a subject or population of subjects that does not have cardiovascular disease (CVD) or subclinical CVD (sCVD). Alternatively, the fold-increase in INT4 cells can be 1.1, 1.2, 1.3, 1.4, 1.5, 2, 5, 10, 20, 25, 50, 75 or 100-fold. The subject is determined to have an increase in the INT4 subset as compared to the statistical sample, where the proportion of the INT4 subset is 12% of a total CD3−CD19−CD14+CD16+CD56− INT population in the normal statistical sample and 15, 20, 25, 30, 35, or 40% of a total CD3−CD19−CD14+CD16+CD56− INT population in the subject. The subject is determined to have an increase in the INT4 subset as compared to the statistical sample where there is roughly one, one and a half, two, three, four or more times the amount INT4 cells present in the subject as compared to the statistical sample. The one or more biomarkers are obtained from a blood, serum, or plasma sample, and the biomarkers are detected as a protein or a nucleic acid.

The INT2 subset is detected by gating the isolated immune cells for IL6RA+, CD69+, CD86+, CD9+, CD141+, CD142+, CD163+, CCR2+, CCR4+, CCR5+, CD36+, CD38+, CD45RO+, CD206+. The INT3 subset is detected by gating the isolated immune cells for IL6RA, CD141+, CD142+, CD163+, CCR2+, CD36+, CD38+, CD69+, CD86+CD9+, CD154, CCR4+, CD69+, and optionally LAG3+, CD206+, CD137+, CCR4+, CCR5+, CD206, CD45RO+, CD4. The INT4 subset is detected by gating the isolated immune cells for IL6RA_{low}, CD137^{lower than INT 2 and 3}, CD154−, CD163^{low}, CCR2^{low}, CCR4^{low}, CCR5^{low}, CD206^{low}, LAG3−, CD36^{lower than INT 2 and 3}, CD38^{low}, and optionally CD45RA, CD9+. The subject is determined to have a decrease in the INT2 subset as compared to the statistical sample, where the proportion of the INT2 subset is 40% of a total CD3−CD19−CD14+CD16+CD56− INT population in the statistical sample and less than 20% of a total CD3−CD19−CD14+CD16+CD56− INT population in the subject.

The subject is determined to have a decrease in the INT2 subset as compared to the statistical sample where there is one half of the amount of INT2 cells present in the subject as compared to the statistical sample. The subject is determined to have a decrease in the INT3 subset as compared to the statistical sample, where the proportion of the INT3 subset is 30% of a total CD3−CD19−CD14+CD16+CD56− INT population in the statistical sample and 10% of the total CD3−CD19−CD14+CD16+CD56− INT population in the subject. The subject is determined to have a decrease in the INT3 subset as compared to the statistical sample where there is one-third of the amount of INT3 cells present in the subject as compared to the statistical sample. The subject is determined to have an increase in the INT4 subset as compared to the statistical sample, where the proportion of the INT4 subset is 12% of a total CD3−CD19−CD14+CD16+CD56− INT population in the statistical sample and 40% of a total CD3−CD19−CD14+CD16+CD56− INT population in the subject. The subject is determined to have an increase in the INT4 subset as compared to the statistical sample where there is roughly four times the amount INT4 cells present in the subject as compared to the statistical sample. The one or more biomarkers are obtained from a blood, serum, or plasma sample, and the biomarkers are detected as a protein or a nucleic acid.

As embodied and broadly described herein, the present disclosure relates to a method of inducing, enhancing, or sustaining an immune response against CVD or subclinical CVD in a subject, the method comprising contacting cells of the subject with an effective amount of an agent capable of treating or preventing cardiovascular disease. In certain embodiments, an immune response against CVD or subclinical CVD is induced, enhanced, or sustained in the subject subsequent to detection in the subject the presence or absence of the biomarkers disclosed herein.

In some embodiments, the detecting in the subject the presence or absence of biomarkers comprises detecting the presence or absence of the cell surface markers, or the activity or expression of their corresponding genes as follows: The subset of lymphocytes consisting essentially of T cells and the one or more biomarkers are selected from 1, 2, 3, 4, 5, 6, 7, 8, 9, 10, 15, 20, 25, 30, 40, or 50 biomarkers selected from: SELPLG; IFITM3; MYC; FYB; JUNB; IL32; SELL; CCR7; GIMAP5; TCF7; FAS; BCL6; TNFSF10; STAT3; CD27; BIRC3; IKZF1; PTPRC; CYTIP; IFITM2; CD2; LCK; IL7R; FOXO1; TRAC; CCA; CD69; ICOS; SLC2A3; LEF1; RGS1; IL4R; CD52; HOPX; GZMA; KLRG1; KLRD1; TIGIT; STAT1; KLRC1; LGALS1; KLRC4; CD63; SAMD3; S100A10; KLRK1; IL23R; CCL5; APOBEC3G; CD160; TARP; CD3D; CD8A; KLRD1; ITGA4; CX3CR1; VNN2; GZMA; CH3L2; or GIMAP5, and optionally the biomarkers are selected in the order listed. In certain embodiments, the expression or activity of one or more of these biomarkers in a subject or sample is measured against a baseline level of expression or activity in a statistical sample, e.g., a sample from a healthy patient. In some embodiments, an increase in the expression or activity of the one or more biomarkers in the subject or sample as compared to the statistical sample is indicative of the patient being at risk of having or developing a cardiovascular disease (CVD) or a subclinical CVD (sCVD) and/or needing treatment for CVD or sCVD. In certain embodiments, the increase in the expression or activity of the biomarkers can be a 1.1×, 1.2×, 1.3×, 1.4×, 1.5×, 2×, 3×, 5×, 10×, 20×, 25×, 50×, 75× or 100× change.

The subset of immune cells consisting essentially of classical monocytes and the one or more biomarkers are selected from 1, 2, 3, 4, 5, 6, 7, 8, 9, 10, 15, 20, 25, 30, or 40 biomarkers selected from: CCL4; CCL3; SLC2A3; SOD2; SELPLG; CD14; LAP3; FYB; LYN; CD80; TNFSF10; TNFSF8; CSF3; CSF3R; CD36; NAIP; SELL; CLEC4E; IL8R; PTPRC; VCAN; ADGRE1; MNDA; DUSP1; FYB; DUSP2; LYZ; JUNB; SCREP1; DOCK8; CD300A; LAP3; TNFSF13; SDCP; MDX1; FYN; STAT6; IL1B; NAMPT; STAT3; IL6; IER3; TLR2; CD83; FYB; S100A9; TNFSF13B; CLEC4E; or ICAM1, and optionally the biomarkers are selected in the order listed. In certain embodiments, the expression or activity of one or more of these biomarkers in a subject or sample is measured against a baseline level of expression or activity in a statistical sample, e.g., a sample from a healthy patient. In some embodiments, an increase in the expression or activity of the one or more biomarkers in the subject or sample as compared to the statistical sample is indicative of the patient being at risk of having or developing a cardiovascular disease (CVD) or a subclinical CVD (sCVD) and/or needing treatment for CVD or sCVD.

In certain embodiments, the increase in the expression or activity of the biomarkers can be a 1.1×, 1.2×, 1.3×, 1.4×, 1.5×, 2×, 3×, 5×, 10×, 20×, 25×, 50×, 75× or 100× change.

The subset of immune cells consisting essentially of intermediate monocytes and the one or more biomarkers are selected from 1, 2, 3, 4, 5, 6, 7, 8, 9, or 10 biomarkers selected from: PRDM1; TNF; CCL4; LYZ; DUSP1; IL1B; DUSP2; CCL3; IER3; or ICAM1, and optionally the biomarkers are selected in the order listed. In certain embodiments, the expression or activity of one or more of these biomarkers in a subject or sample is measured against a baseline level of expression or activity in a statistical sample, e.g., a sample from a healthy patient. In some embodiments, an increase in the expression or activity of the one or more biomarkers in the subject or sample as compared to the statistical sample is indicative of the patient being at risk of having or developing a cardiovascular disease (CVD) or a subclinical CVD (sCVD) and/or needing treatment for CVD or sCVD. In certain embodiments, the increase in the expression or activity of the biomarkers can be a 1.1×, 1.2×, 1.3×, 1.4×, 1.5×, 2×, 3×, 5×, 10×, 20×, 25×, 50×, 75× or 100× change.

The subset of lymphocytes consisting essentially of B cells and the one or more biomarkers are selected from 1, 2, 3, 4, 5, 6, 7, 8, or 9 biomarkers selected from: CD74; IGHD; CD79A; LYN; CD83; R3HDM4; CD79B; CXCR5; or SLC2A3, and optionally the biomarkers are selected in the order listed. In certain embodiments, the expression or activity of one or more of these biomarkers in a subject or sample is measured against a baseline level of expression or activity in a statistical sample, e.g., a sample from a healthy patient. In some embodiments, an increase in the expression or activity of the one or more biomarkers in the subject or sample as compared to the statistical sample is indicative of the patient being at risk of having or developing a cardiovascular disease (CVD) or a subclinical CVD (sCVD) and/or needing treatment for CVD or sCVD. In certain embodiments, the increase in the expression or activity of the biomarkers can be a 1.1×, 1.2×, 1.3×, 1.4×, 1.5×, 2×, 3×, 5×, 10×, 20×, 25×, 50×, 75× or 10× change.

In alternative embodiments, the detecting in the subject the presence or absence of biomarkers comprises detecting the presence or absence of the cell surface markers, or the activity or expression of their corresponding genes. In further embodiments, the detecting in the subject the presence or absence of biomarkers comprises detecting the presence or absence of the cell surface markers, or the activity or expression of their corresponding genes.

In one non-limiting embodiment, the contacting includes administrating the effective amount of the composition to the subject.

In one non-limiting embodiment, the herein described biological sample can be obtained by any known technique, for example by drawing, by non-invasive techniques, or from sample collections or banks, etc.

In one non-limiting embodiment, the herein described methods and/or kits may employ, for example, a dipstick, a membrane, a chip, a disk, a test strip, a filter, a microsphere, a slide, a multi-well plate, an optical fiber, and the like, or any other variant available to the person skilled in the art without departing from the present disclosure. For example, a test strip may be used where a sample to be tested can be added dropwise to a sample application pad present on the test strip, and the presence or absence of at least one or more biomarker disclosed herein is made based on an immunodetection method which detects presence of the at least one such biomarker. Such immunodetection method may include an immunochromatographic test, an ELISA or ELISPOT or variant thereof, and the like, or any other suitable method available to the person skilled in the art without departing from the present disclosure.

As embodied and broadly described herein, the present disclosure also relates to a kit comprising of reagents capable of detecting a biomarker or combination of the biomarkers disclosed in the present disclosure and instructions for use. The instructions for use may be to implement any one of the herein described methods, for example subsequent administration of a therapeutic or preventative vaccination against CVD.

As embodied and broadly described herein, the present disclosure further relates to an in vitro method for detecting an CVD or subclinical CVD in a subject. The method comprises providing a biological sample from the subject, the biological sample comprising cells from the subject. The method further comprises contacting the sample with a composition of the present disclosure, wherein the composition is capable of detecting a biomarker or combination of the biomarkers disclosed in the present disclosure. The method also comprises processing the sample to detect the presence of the biomarkers, and detecting the presence or absence of the biomarkers. The presence of the biomarkers being indicative that the subject has CVD or subclinical CVD. The method may further include causing a transmission of electronic notification data conveying information indicative of whether the subject has tested positive for such biomarkers, or CVD or subclinical CVD.

In one non-limiting embodiment, the electronic notification data is transmitted to a computing device associated with a particular user, which can be a medical expert or the subject. In some specific practical implementations, the computing device associated with the particular medical expert may include a smartphone, a tablet, a general purpose computer and/or any other suitable computing device and the electronic notification data may convey an e-mail message, an SMS message and/or or any other suitable electronic message.

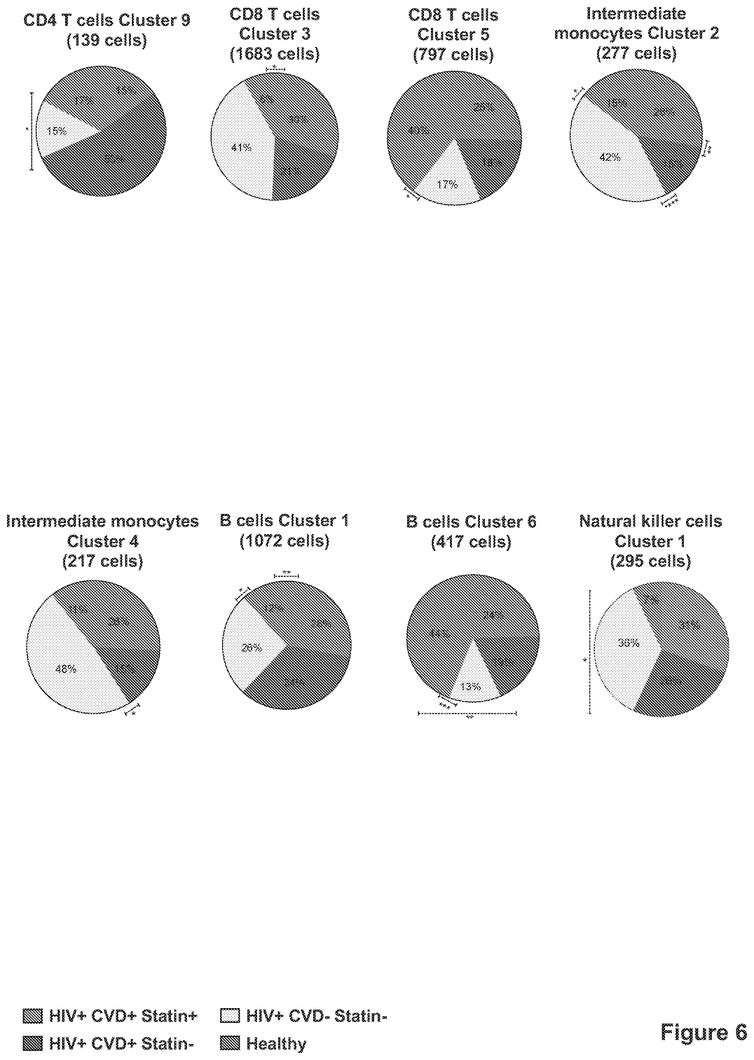

In one non-limiting example, people living with HIV have a significantly increased risk of cardiovascular disease (CVD) due to atherosclerosis. The Women's Interagency HIV Study (WIHS) collected peripheral blood mononuclear cells (PBMCs) from women living with HIV and data on subclinical CVD defined by carotid artery B mode ultrasound, viral load and many clinical parameters. Disclosed herein, 32 multiplexed PBMC samples using single cell (sc) RNA sequencing combined with cellular indexing of transcriptional epitope sequencing (CITE-Seq) of women with 1) HIV, 2) HIV and CVD, 3) HIV and CVD treated with cholesterol-lowering drugs and 4) women without HIV or CVD. Expression of 40 surface markers enabled detailed analysis of all major known cell types (CD4 T cells, CD8 T cells, B cells, NK cells, classical, intermediate and nonclassical monocytes), yielding unprecedented resolution (50 distinct clusters) in almost 42,000 single cells. The cell number in eight of these clusters showed statistically significant differences among the 4 groups, including one CD4 T cell, 2 CD8 T cell, 2 B cell, 2 intermediate monocyte and 1 NK cell cluster (FIG. 6). Identity and function of these cell types was inferred from both surface markers and transcriptomes. The PBMC biomarkers discovered in this disclosure are defined by unique combinations of cell surface proteins. Thus, these new biomarkers and effects of preventative or therapeutic interventions can be monitored by inexpensive flow cytometry in readily available blood samples. The inventor hereby incorporates by reference the publication entitled, “Combined protein and transcript single cell RNA sequencing reveals cardiovascular disease and HIV signatures”, published in BioRxiv, Sep. 12, 2020, www.biorxiv.org/content/10.1101/2020.09.10.292086v1.full, and all text, figures, tables and supplemental information therewith, in its entirety.

The biomarker cell subsets were discovered by BD Rhapsody. Embodiments of the present disclosure are directed to a method that provides information on the surface phenotype and transcriptome of many single cells. Specifically, 496 genes were examined using 40 antibodies. 41,661 cells passed all quality controls and were successfully analyzed.

To understand which cell types might be informative for the effects of living with HIV (HIV), cardiovascular disease (CVD) and management of cardiovascular disease (LDL cholesterol medication), the inventors first compared the proportions (cell percentages) for each of the 50 clusters. Statistical significance was determined by log odds ratio and ANOVA.

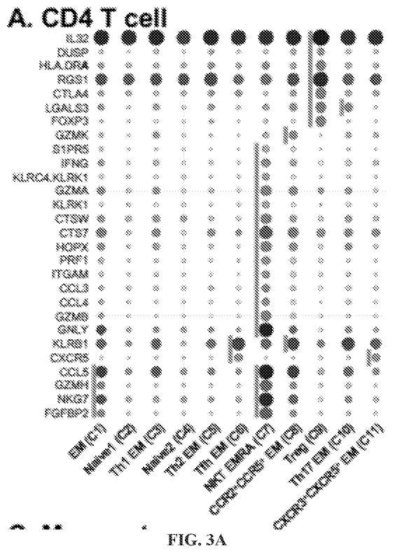

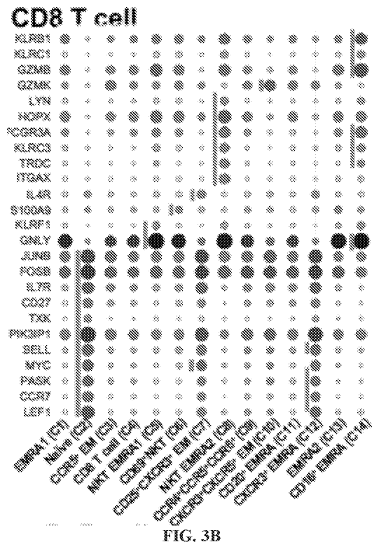

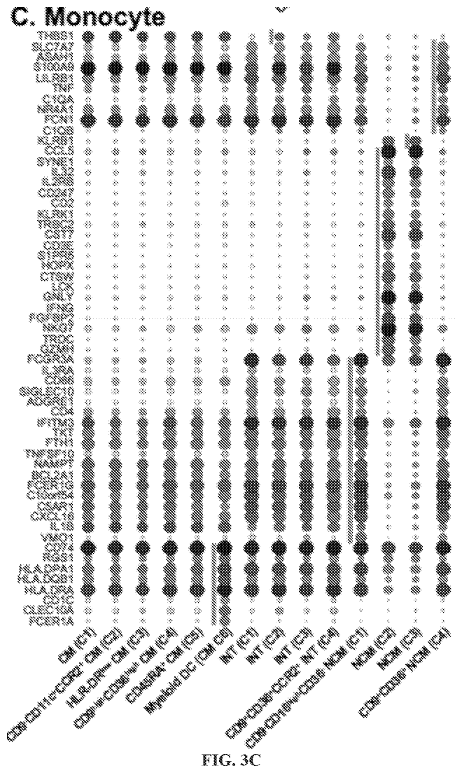

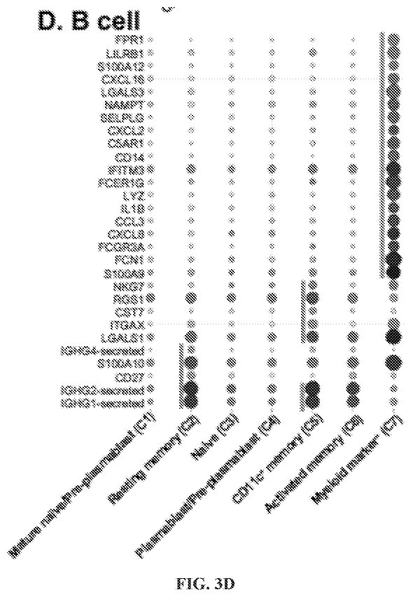

Three comparisons are most informative: healthy vs HIV+ (reflecting the HIV signature), HIV+ vs HIV+CVD+ (reflecting the CVD signature), and HIV+CVD+ vs HIV+CVD+ treated with LDL cholesterol-lowering drugs (reflecting the cholesterol control signature). FIGS. 3A-H show the proportions of cells for those clusters in which at least one group was significantly different from the other three (significance level indicated). Below the proportions, the expression of all cell surface markers is shown as a violin plot for each of the seven clusters that showed significant differences.

CD4 T cell cluster 9 (C9) was significantly elevated in women with sCVD. Cluster 9 contains Tregs. Early in atherosclerosis, Tregs are known to be elevated, which is consistent with a subclinical CVD diagnosis based on carotid ultrasound. Notably, these women (HIV+CVD+) had no clinical symptoms, consistent with very early atherosclerosis. CD8 T cell cluster 3 (C3) has a significantly higher cell proportion in participants that are HIV+CVD+ and receive cholesterol-lowering drugs compared to healthy. Cluster 3 contains CCR5+ effector memory CD8 T cells. CD8 T cell cluster 5 (C5) cells are significantly more abundant in healthy individuals than in individuals with HIV. These are NKT cells. Intermediate monocyte clusters 2 and 4 (C2, C4) are much more abundant in individuals with HIV than in healthy women. Cluster 2 (C2) returns to a proportion identical to healthy in women with HIV who also have sCVD. Remarkably, this is significantly reversed by treatment with cholesterol-lowering drugs. Cluster 4 (C4), which is tissue factor (CD142) positive, shows a very similar pattern. B cell clusters 1 and 6 (FIGS. 3F, 3G, respectively) show diametrically opposite patterns: cluster 1 cells increase in individual with HIV and cluster 6 cells decrease. Cluster 1 B cells further increase with CVD and this difference becomes significant from healthy in individuals who also receive cholesterol-lowering drugs. Finally, NK cell cluster 1 (FIG. 3H) was increased in patients HIV+CVD+ compared to healthy participants. The overall percentages of these cell types by population are shown in FIG. 6.

The inventors have found transcriptomes and cell surface phenotypes, disclosed herein, of almost peripheral blood mononuclear cells (PBMCs) using targeted scRNA-Seq (BD Rhapsody platform), simultaneously providing surface phenotype and transcriptomes in the said cells. PBMCs from 32 subjects were studied using 40 oligonucleotide-tagged monoclonal antibodies; 485 transcripts were amplified.

All features of exemplary embodiments which are described in this disclosure and are not mutually exclusive can be combined with one another. Elements of one embodiment can be utilized in the other embodiments without further mention. Other aspects and features of the present invention will become apparent to those ordinarily skilled in the art upon review of the following description of specific embodiments in conjunction with the accompanying figures.

### EXAMPLES

### Example 1: Population and Cells

The 32 participants in the Women's Interagency HIV Study (WIHS) were aged 40-74, all (ex)smokers, most black or Hispanic. Matched groups of 8 women each were selected for sCVD and HIV status: 1) sCVD-HIV+(living with HIV); 2) sCVD+HIV+statin− (living with HIV, evidence of CVD); 3) sCVD+HIV+statin+ (living with HIV, evidence of CVD, treated with cholesterol-lowering drugs) and 4) sCVD-HIV− (healthy). PBMC tubes were shipped from the central repository on liquid N2, thawed and processed according to standard operating procedures (see methods). Cell viability was 88±5%. To avoid batch effects, all cells were hash-tagged for multiplexing, with 4 samples run per 250,000-well plate (total of 8 plates). The pooled cells were labeled with 40 titrated oligonucleotide-tagged mAbs. After quality controls and three-stage doublet removal (see methods), 41,611 transcriptomes of single cells from 31 WIHS participants (one sample was lost in hashtagging) were successfully analyzed.

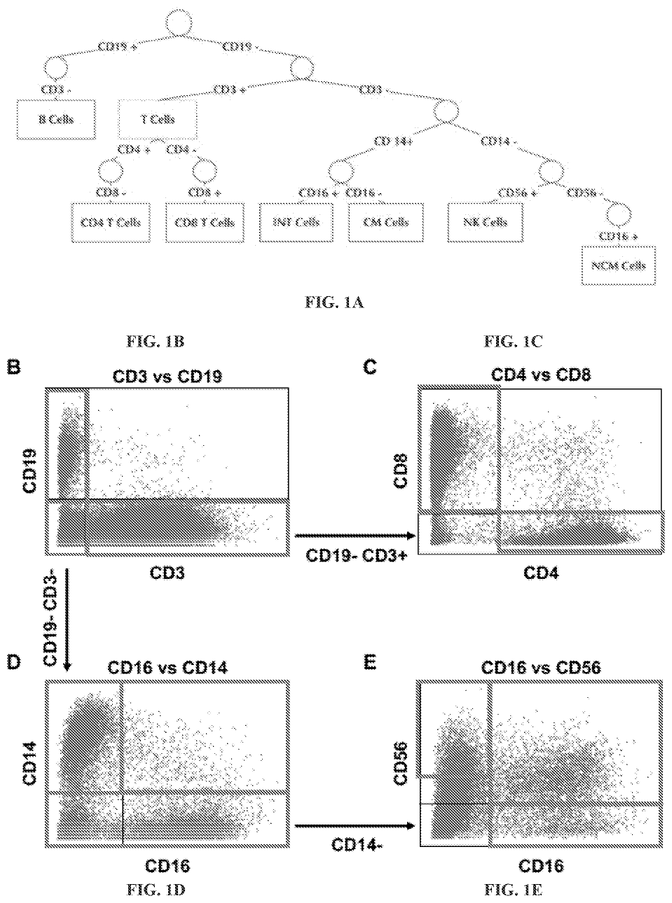

Cell types. First, known and abundant cell types were identified by 8 antibody markers using biaxial gating, corresponding to established gating schemes for human PBMCs.³⁹⁻⁴¹ CD3 and CD19 expression are mutually exclusive and specific for T and B cells, respectively (FIG. 1A). Thus, B cells were identified as CD19+CD3− and T cells as CD19−CD3+(FIG. 1B). Among the CD3+CD19− T cells, CD4 T cells were identified as CD19−CD3+CD4+CD8− and CD8 T cells as CD19−CD3+CD4−CD8+ (FIG. 1C). From the CD19−CD3− cells, standard monocyte gating by CD14 and CD1642,43 was used to identify monocytes (FIG. 1D). Classical monocytes (CM) were identified as CD3−CD19−CD14+CD16− and intermediate monocytes as CD3−CD19−CD14+CD16+. The CD16+ cells also contain NK cells, which were identified by CD56 and defined as CD56+CD14−CD20−CD123−CD206− (FIG. 1E).44 The remaining CD3−CD19−CD56−CD16+ cells were identified as nonclassical monocytes.

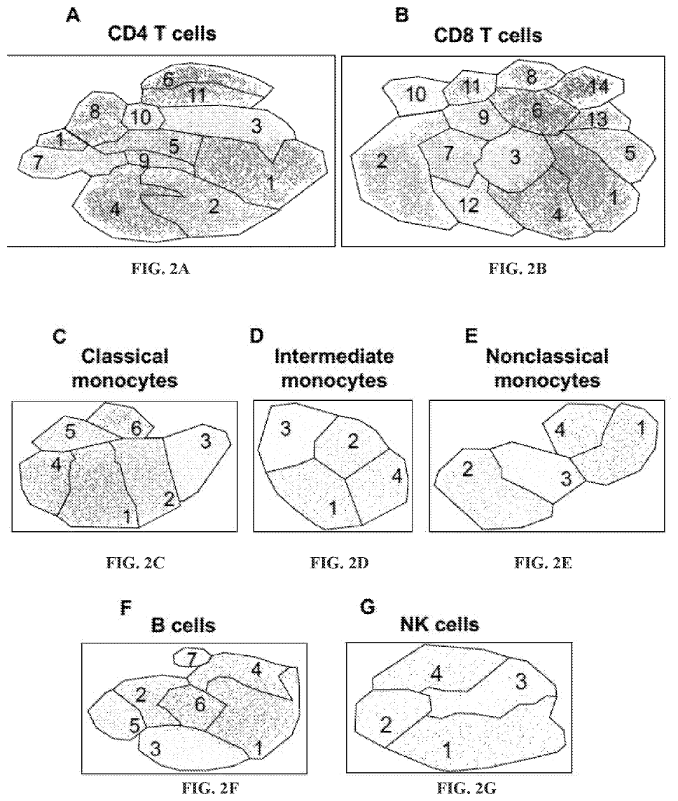

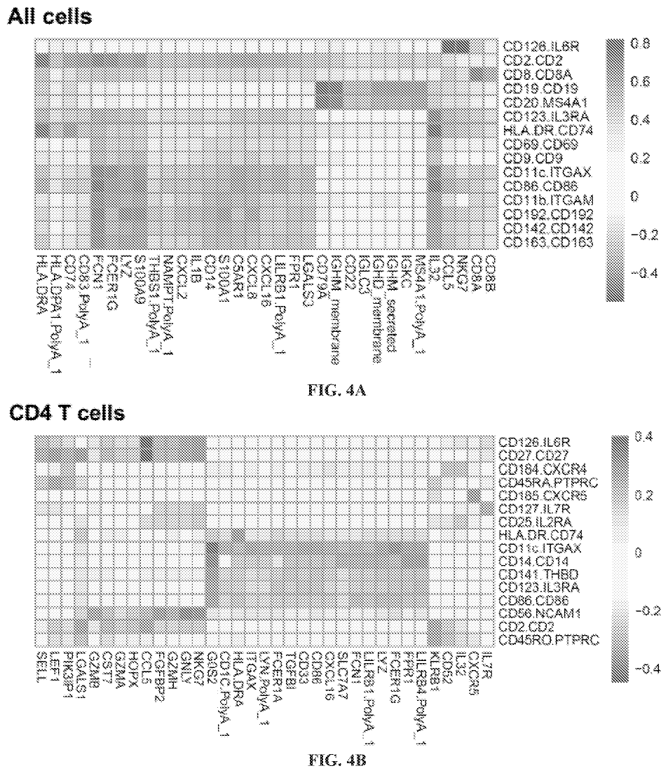

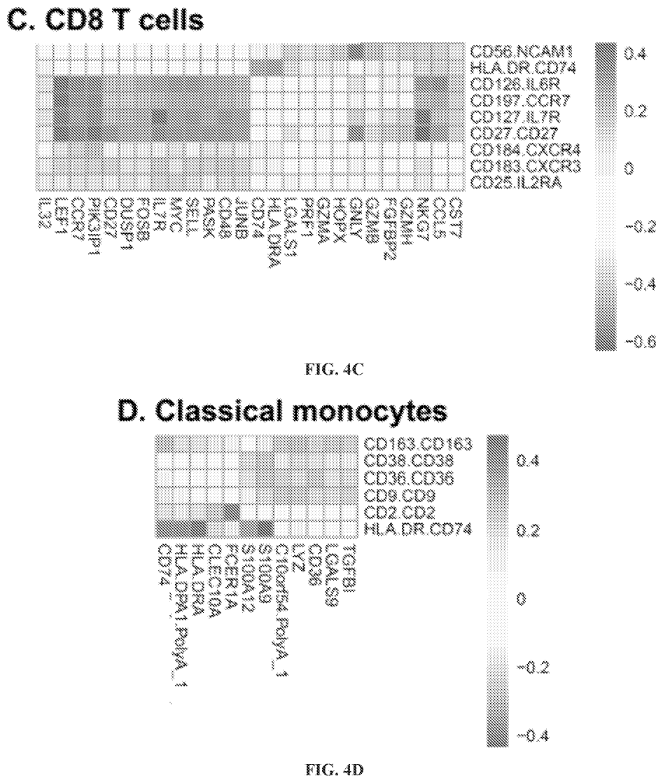

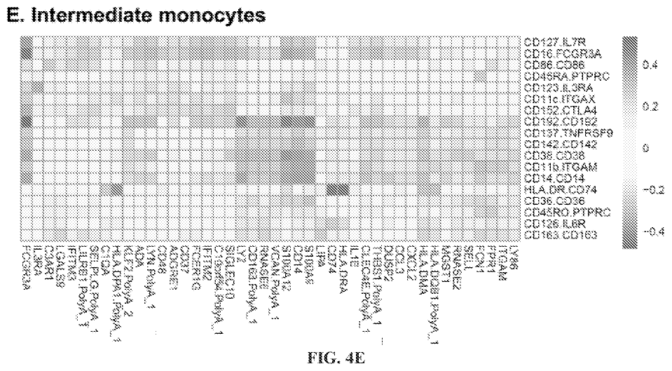

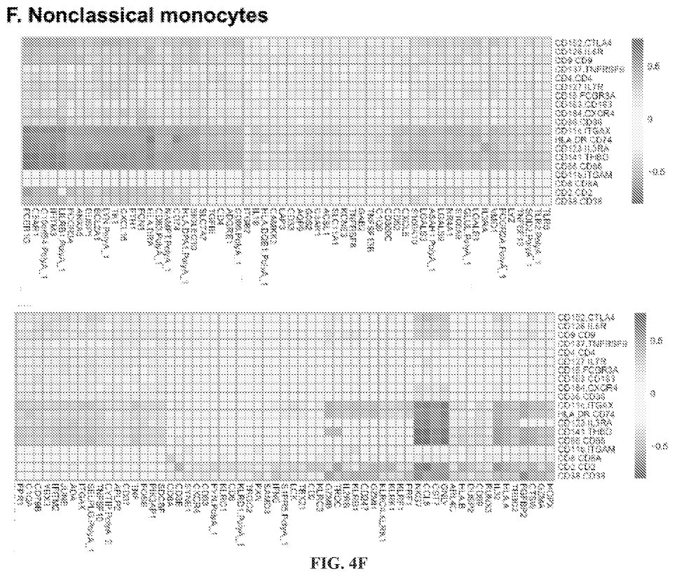

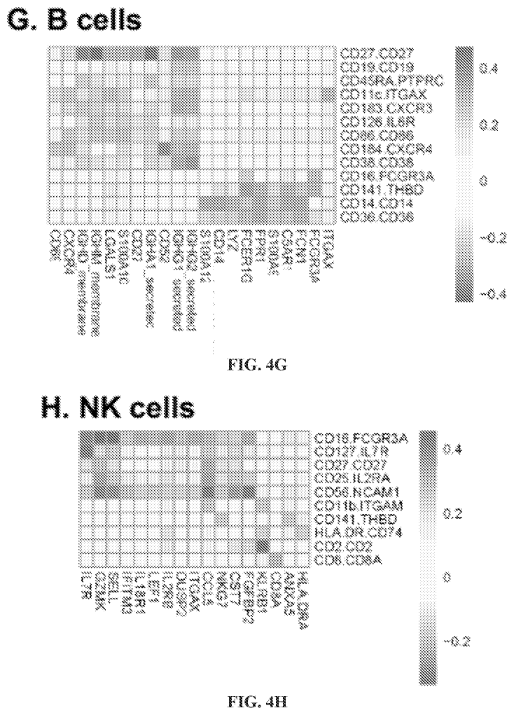

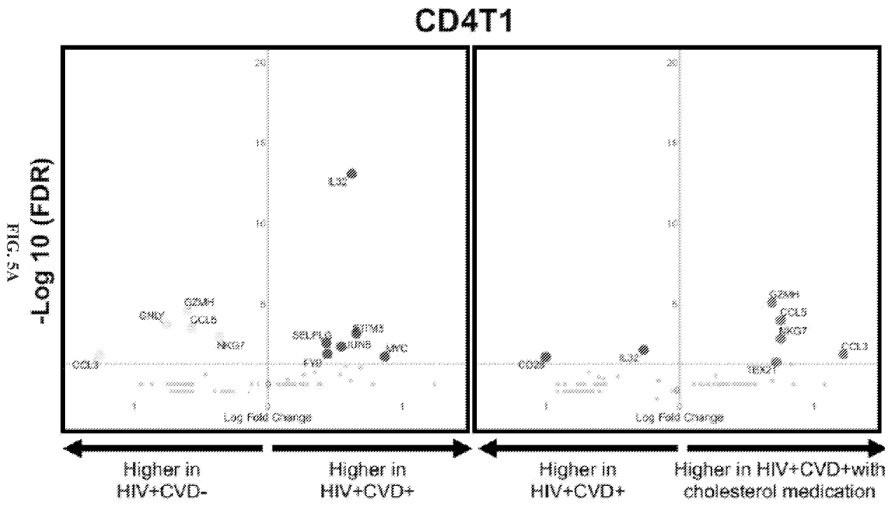

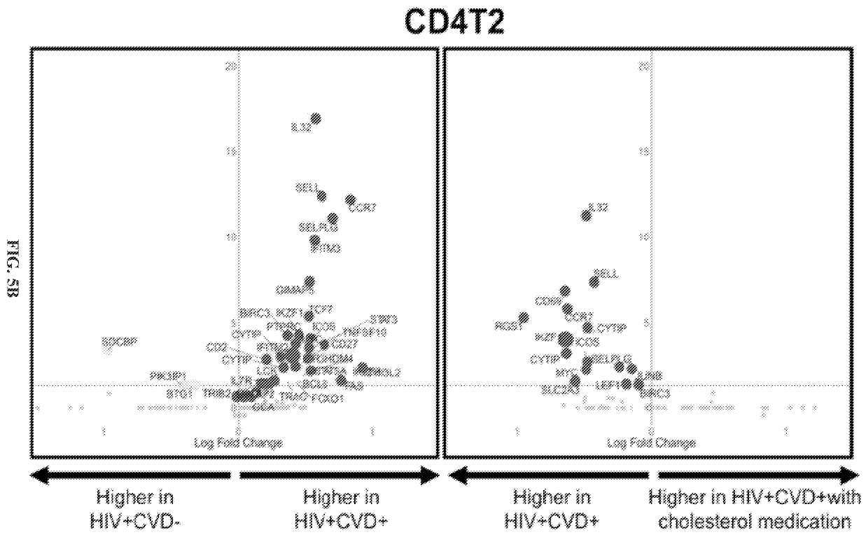

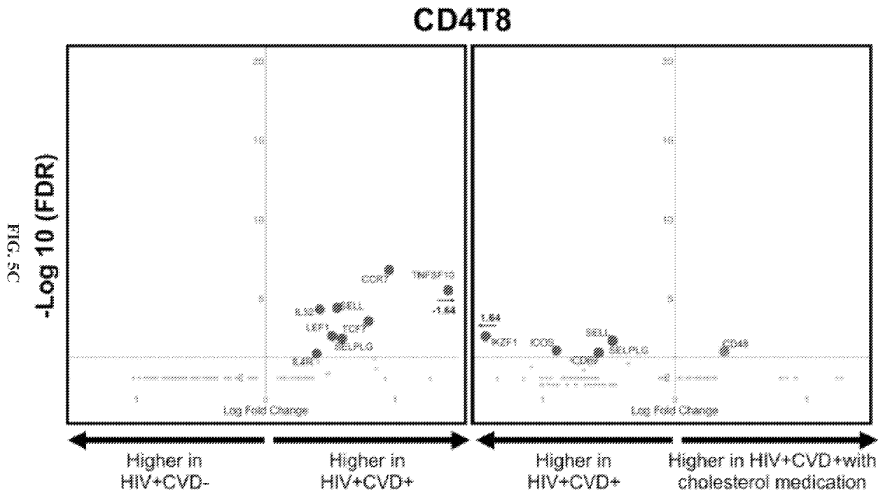

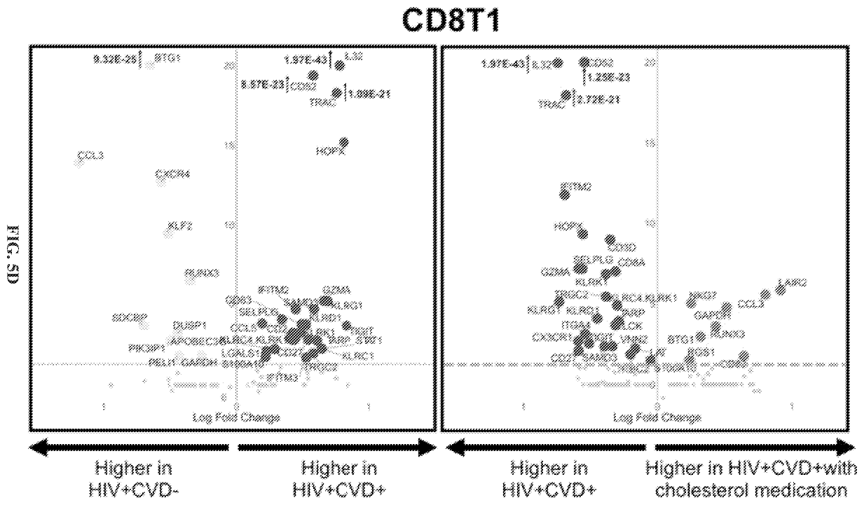

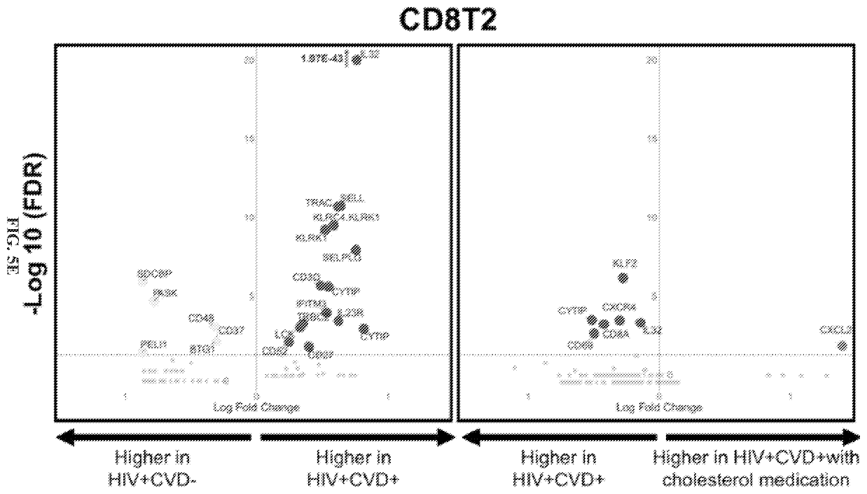

This resulted in unequivocal identification of 7 well-known PBMC leukocyte types (CD4 T, CD8 T, B, NK cells and classical, intermediate and nonclassical monocytes). Each of these cell types were clustered into a UMAP using all non-negative antibodies, followed by Louvain45 clustering (FIGS. 2A to 2G). Gates were overlaid for clarity and used in subsequent figures where antibody or gene expression is shown. In total, 11 CD4 T cell clusters (FIG. 2A), 14 CD8 T cell clusters (FIG. 2B), 6 classical (FIG. 2C), 4 intermediate (FIG. 2D) and 4 nonclassical monocyte (FIG. 2E) clusters, 7 B cell clusters (FIG. 2F) and 4 NK cell clusters (FIG. 2G) were analyzed.

### Example 2: PBMC Subsets and Clusters

The expression of all 40 surface markers was projected onto the 7 UMAPs, retaining the gates from FIG. 2A to 2G for clarity. Log 2 (expression) was encoded from 0 (=no expression) to the maximum for each antibody, resulting in 280 UMAPs. Most clusters could be called based on the expression of cell surface molecules, using known T, B, NK and monocyte subsets.

Among CD4 T cells, CD2 was expressed in almost all cells, but at levels that varied over a factor of 4. The high affinity IL2 receptor IL2RA (CD25) was expressed in about a third of the CD4 T cells and was strikingly high in cluster 9. Cluster 9 was negative for IL7 receptor (CD127), identifying cluster 9 as regulatory T cells (Tregs). The TNF superfamily member CD27 was broadly expressed. CD45RA and RO were mutually exclusive, identifying naive and antigen-experienced CD4 T cells. CD56 was only expressed in cluster 7, identifying these cells as CD4+ NKT cells.46 IL-6R (CD126) and IL-7R (CD127) were broadly expressed in more than half of the CD4 T cells. CD152 (CTLA4) was expressed in about one fifth of CD4 T cells, including in Tregs. CXCR3 (CD183) identifies T-helper cells in human PBMCs47 and was highly expressed in clusters 3 and 11. Cluster 11, but not 3, co-expressed CXCR5 (CD185). Cluster 6 expressed CXCR5 as the only chemokine receptor, identifying it as putatively containing follicular helper (TFH) T cells. CCR2 and CCR5, which are thought to identify highly activated T cells, were only expressed in cluster 8. CCR4, typical of Th2 cells, was expressed in cluster 5. CCR7 expression in cluster 4 identifies these cells as naive, because they also express CD45RA. Based on this information, all CD4 T cell clusters were called.

Like in CD4 T cells, almost all CD8 T cells expressed CD2. Cluster 6 exclusively expressed CD9 and CD36. Together with CD69 expression, this identifies cluster 6 as CD8+ NKT cells.48,49 Cluster 8 exclusively expressed CD11c, showing NKT cells with a CD45RA+ memory (TEMRA) phenotype. CD25 was exclusively limited to cluster 7, whereas CD27 was expressed in about half of the CD8 T cells. CD45RA and RO were mutually exclusive. CD56 expression identifies cluster 5 as NKT cells. IL-6R (CD126) and IL-7R (CD127) were largely co-expressed in CD8 T cells, as were CD137 (4-1BB) and CD152 (CTLA-4). CXCR5 was exclusively expressed in cluster 10, which carries other effector memory markers and also expresses CXCR3. CCR7 expression identifies cluster 2 as naive CD8 T cells. Based on these markers, all CD8 T cell clusters but cluster 4 were called.

Classical monocytes formed 6 clusters. All were CD11b+. There were gradients of CD9, CD69, CD137, CD142 (tissue factor), CD152 and CD163 (hemoglobin-haptoglobin receptor) expression, being highest in cluster 4 and lowest in cluster 3. The CD9 gradient has previously been described in a mass cytometry study.42 CD36 and CD86 were expressed in all classical monocytes. IL-7R (CD127) was very high in about half of the cells in cluster 4. CXCR3 was only expressed in cluster 4. Expression of this receptor has been controversial.50,51 Finding it in only a small subset may help resolve this controversy. CXCR4 and CCR4 were expressed in about half of the classical monocytes. Based on these markers, 5 of the 6 classical monocyte subsets were called and related to subsets described by mass cytometry.42

Intermediate CD14+CD16+ monocytes are considered pro-inflammatory by many investigators.52,53 Indeed, they all expressed the inflammation-induced costimulatory molecule CD86. Cluster 4 highly expressed CD142 (tissue factor), which has previously been implicated in people living with HIV.37 CD137 formed a gradient, highest in intermediate monocyte cluster 4 and lowest in cluster 3. Intermediate monocytes formed 4 clusters. Since subsets of intermediate monocytes have not been described before, this is a new finding that awaits confirmation by future studies.

Nonclassical monocytes have specialized roles and are considered anti-inflammatory by some investigators.54 They formed 4 clusters. Strikingly, CD11c, CD86, CD141, CD152, CD183 (CXCR3) and HLA-DR (CD74) were confined to clusters 1 and 4. MHC-II+ nonclassical monocytes have been described before.42 Expression of CD9, CD36 and CXCR4 was limited to cluster 4, providing that this cluster corresponds to the previously described CD9+CD36+ nonclassical monocytes.42

CD20 was expressed in all B cells, although at different levels. Cluster 1, based on the high expression of CD38 could potentially be a pre-plasmablast,⁵⁵ but also has features of naive B cells (CD27− and positive for IgM and IgD transcripts, (not shown)).^{55,56} Cluster 2 B cell expresses CD27 and CXCR3, indicating a resting memory B cell. Cluster 3 has marker expression consistent with naïve B cells. Cluster 3 is negative for CD27 with high transcript expression of IgM and IgD (not shown). Cluster 4 is another cluster with CD38+ expression. However, there is a partial part of this cluster with CD27+ expression. Therefore, it is likely that cluster 4 is a mixture between pre-plasmablasts (CD38+CD27−) and plasmablasts (CD38+CD27+).^{55,56} CXCR4 was confined to clusters 1, 4 and 7. CXCR4 in B cells has recently received attention as a possible marker of anti-atherosclerotic B cells.⁵⁷ All B cells express CD74 (the MHC-II molecule HLA-DR), identifying them as antigen-presenting cells. In B cells, CD137 (4-1BB) was broadly expressed. Almost all B cells except cluster 5 expressed CXCR5. Cluster 5 is negative for CD27 and expresses CXCR3, CCR6 and high CD11c which could possibly be a CD11c+ memory B cell. CD11c+ B cells increase in number in HIV infected patients as well as patients with autoimmune disease (SLE, RA, CVID), but mechanistic roles of these B cells in these disease processes remain unclear.³⁴ B cell cluster 6 expressed CD25 which is a known marker for activation for B cell proliferation and exhaustion.^{58,59} It also expresses CD69 and CD27 representing probably an activated memory B cell. Cluster 7 expressed CD36 and other myeloid markers. However, these cells were gated on CD19+CD3−, identifying them as bona fide B cells. Cluster 7 also expressed CD20. Based on these markers, 6 of the 7 B cell clusters were called.

NK cells were clearly separated into immature (CD56brightCD16−, cluster 4) and mature (CD56dim/CD16+, clusters 1, 2 and 3) subsets. Cluster 3 was CD11b+ and parts of cluster 3 were also CD11c+. A few CD11c+ cells were also found in cluster 4, as were some CD25+ and CD27+ immature NK cells. CD38 was expressed in some immature NK cells and confined to cluster 3 of the mature NK cells. CD137 (4-1BB) was found at modest levels on about half of the NK cells. A few NK cells express CD142 (tissue factor), identifying them as pro-thrombotic. CD152 (CTLA-4) was expressed at moderate levels in more than half of the NK cells. A few NK cells in cluster 1 expressed CD195 (CCR5), some of them at high levels.

### Example 3: Biomarker PBMC Subsets

The present disclosure provides that CD4 T cells are significantly reduced in people living with HIV (median [25-75 percentile]: 41% [31-53%] of all T cells compared to 66% [59-72%] in healthy participants). To understand which cell types might be informative for understanding the effects of living with HIV (HIV), cardiovascular disease (CVD) and management of cardiovascular disease (LDL cholesterol medication), the proportions (cell percentages) for each of the 50 clusters were compared. Statistical significance was determined by log odds ratio and ANOVA.

Three comparisons are most informative: healthy vs HIV+ (reflecting the HIV signature), HIV+ vs HIV+CVD+ (reflecting the CVD signature), and HIV+CVD+ vs HIV+CVD+ treated with LDL cholesterol-lowering drugs (reflecting the cholesterol control signature). The proportions of cells for those clusters in which at least one group was significantly different from the other three. Below the proportions, the expression of all cell surface markers is shown as a violin plot for each of the eight clusters that showed significant differences.

CD4 T cell cluster 9 was significantly elevated in women with sCVD. Cluster 9 contains Tregs. Early in atherosclerosis, Tregs are known to be elevated,62-64 which is consistent with a subclinical CVD diagnosis based on carotid ultrasound. Notably, these women (HIV+CVD+) had no clinical symptoms, consistent with early atherosclerosis. CD8 T cell cluster 3 has a significantly higher cell proportion in participants that are HIV+CVD+ and receive cholesterol-lowering drugs compared to healthy. Cluster 3 contains CCR5+ effector memory CD8 T cells. CD8 T cell cluster 5 cells are significantly more abundant in healthy individuals than in individuals with HIV. These are likely NKT cells. Little is known about CD8 NKT cells in HIV.

Intermediate monocyte clusters 2 and 4 are much more abundant in individuals with HIV than in healthy women. Cluster 2 returns to a proportion similar to healthy in women with HIV who also have sCVD. Remarkably, this is significantly reversed by treatment with cholesterol-lowering drugs. Cluster 4, which is tissue factor (CD142) positive, shows a very similar pattern. B cell clusters 1 and 6 show diametrically opposite patterns: cluster 1 cells increase in individual with HIV and cluster 6 cells decrease.32 Cluster 1 B cells further increase with CVD and this difference becomes significant from healthy in individuals who also receive cholesterol-lowering drugs. Finally, NK cell cluster 1 was increased in patients HIV+CVD+ compared to healthy participants.

### Example 4: Gene Expression

Next, gene expression in each cluster was analyzed and it was determined which genes were significantly differentially expressed from all other clusters of the respective cell type. The average single cell transcriptomes are represented by dot plots (FIGS. 3A to 3D), where the size of each dot indicates the percentage of positive cells for that gene in the respective cluster and the color shows its log 2 expression level. The lines highlight the genes that were significantly overexpressed in the respective cell type. The log 2 values, percentages and p-values for all genes in each of the cell types was determined.

All CD4 T cells highly expressed IL32, but it was highest in Tregs (cluster 9) (FIG. 3A). Other genes overexpressed in Tregs include CTLA4, RGS1, and the hallmark transcription factor FOXP3. The CD4 T cells with the most distinct transcriptome were the NK T cells (cluster 7). They significantly overexpressed typical NKT genes including GZMH, GZMA, NKG7 and KLRC4, but also many pro-inflammatory chemokines including CCL3, CCL4 and CCL5 and the Th1 cytokine IFNG (interferon-γ). Effector memory CD4 T cells (cluster 1) also overexpressed CCL5, but not CCL3 or CCL4. A cluster identified as effector memory TFH (cluster 6) significantly overexpressed the TFH-characteristic chemokine receptor CXCR5 and KLRB1. A second cluster of CXCR5+ CD4 T cells (cluster 11) also overexpressed CXCR3, the hallmark chemokine receptor of Th1 cells. The subset of CCR5+CCR2+ effector memory CD4 T cells (cluster 8), likely highly activated, overexpressed KLRB1 and GZMK. Th17 overexpressed LGALS3, which encodes galectin-3, a known biomarker for CVD.65,66

Among CD8 T cells (FIG. 3B), the naive subset had the most significantly different transcriptome from the other subsets. They overexpress the transcription factor subunits JUNB and FOSB, IL7R and CD27. Typical naive T cell (cluster 2) markers significantly overexpressed in their transcriptome are the chemokine receptor CCR7 and the adhesion molecule SELL (L-selectin). They also overexpress the PI3 kinase PIK3IP1. Among CD8 T cells, three clusters of NKT cells were found (cluster 5, 6 and 8), all with an effector memory phenotype. Cluster 5 significantly overexpressed GNLY and KLRF1, and cluster 8 significantly overexpressed the Fc receptor FCGR3A, KLRC3 and the integrin ITGAX (CD11c). A third NKT cell subset (cluster 6) was CD69+ and significantly overexpressed S100A9, which is also found in neutrophils. A CD16+ effector memory subset of CD8 T cells (cluster 14) significantly overexpressed KLRB1 and KLRC1, GZMB, FCGR3A, which encodes one isoform of CD16, and GNLY. An unexpected CD20+ CD8 T cell subset with an effector memory phenotype significantly overexpressed PASK and CCR7.

Among monocytes (FIG. 3C), the most interesting gene signatures were seen in the nonclassical monocytes (4 clusters). The CD9−CD16hiCD36− subset (cluster 1) significantly overexpressed many pro-inflammatory genes including IL1B encoding IL-1β, C5AR1 (CD88), TNFSF10 as well as the antigen presenting genes CD74 and CD86. A second cluster of nonclassical monocytes (cluster 2) was characterized by significant overexpression of CCL5, IL32 and IFNG (interferon-γ). The CD9+CD36+ cluster of nonclassical monocytes (cluster 4) overexpressed S100A9, TNF and C1Q. The myeloid DC cluster significantly overexpressed CD74, CD1C and the MHC-II genes HLADP, HLADQ and HLADR.

Among the B cells (FIG. 3D), the CD27+CXCR3+ subsets (cluster 2) significantly overexpressed IGHG1, IGHG2 and IGHG4, encoding IgG1, 2 and 4, as well as S100A10. Plasma cells significantly overexpressed LGALS1 encoding galectin-1, ITGAX encoding CD11c, RGS1 and NKG7. Cluster 6, although clearly a B cell (CD19+CD3−) expressed many myeloid genes. Among NK cells, cluster 4 significantly overexpressed GZMH.

### Example 5: Correlation Between Gene and Cell Surface Marker Expression

In immunology, surface markers are widely used to define and distinguish cell types.⁶⁷⁻⁶⁹ Arguably, flow cytometry, which measures expression of surface markers, is the discipline-defining method of immunology.⁷⁰ Many surface markers are composed of just one gene product, so it can be expected that the expression level of each gene would correspond to the expression of its corresponding surface marker.

However, cell surface proteins have a long journey from mRNA to expression. They must reach the Golgi, be glycosylated, packaged into vesicles, and reach the plasma membrane. Many cell surface proteins are also modified by proteases and other enzymes.⁷⁰ The antibodies used for detecting surface markers are monoclonal, which means they see just one epitope. Different isoforms of surface markers exist, which could be caused by splice variants,⁷¹ differential glycosylation⁷² or differential processing.⁷³ Finally, the stability and turnover of cell surface proteins varies widely and is controlled by transporters, fusion molecules and often other cell surface molecules.⁷⁴ Thus, the correlation between cell surface protein and mRNA expression is generally quite weak.⁷⁵ scRNA-Seq without CITE-Seq tries to identify all cell types based on mRNA. This has led to much frustration in the field.^{67,76,77} In fact, it is difficult to call cell types based on gene expression data.

Gene expression with cell surface expression was correlated for 41 pairs of genes and proteins. The best correlations between gene and surface protein expression was found for MHC-II. The signal from the antibody to CD74 was very highly correlated with the expression of both the CD74 and the HLA-DR genes. This was true for all cells (correlation coefficient r=0.48; 0.48), but also in each individual cell type, especially in monocytes (r=0.50; 0.51 for intermediate, 0.39; 0.49 for classical, 0.31; 0.39 for nonclassical), CD4 T cells (r=0.39; 0.44). The correlation was somewhat lower for CD8 T cells (r=0.25; 0.20) and low for B cells (r=0.09; 0.12) and NK cells (r=−0.01; 0.21).

CD141 surface expression was highly correlated with the THBD gene expression in intermediate monocytes (0.56). In intermediate monocytes, CD16 protein and gene expression were well correlated (0.42), as were CD123 and IL3RA (0.32), CD127 and IL7RA (0.32). Interestingly, this was not the case in classical or nonclassical monocytes. Across all cells, CD4 surface and gene expression were reasonably well correlated (0.30). CD8 surface expression was highly correlated with CD8A, but not B gene expression in nonclassical monocytes (0.45).

Thus, less than half of the surface markers measured are reasonably well correlated with the mRNA of their encoding genes. This illustrates how CITE-Seq adds enormous value to sc-RNA-Seq, because it fills in the missing information for the half of the surface markers that cannot be predicted by message.

### Example 6: Matrix Correlation Among all Genes and Surface Markers

Since more half of the cell surface markers cannot be predicted from the expression of their encoding genes, other correlations were examined to find surrogate markers. Such markers can be used in experiments where information on cell surface expression is not available. All correlations between all genes and all surface markers were calculated and filtered for those that were statistically (Spearman Correlation test) significant and whose |r|>0.25 for the individual cell types (0.5 for all cells pooled).

Across all cells (FIG. 4A), FCN1 gene expression is highly correlated with CD11c and CD86 surface expression (r=0.8) and well correlated with CD11b, CD172 (CCR2), CD142 (tissue factor) and CD163 (hemoglobin-haptoglobin receptor). Also, IGHM membrane and CD79A gene expression are excellent surrogates for CD19 and CD20 cell surface expression, because they all define B cells. Other immunoglobulin genes are also well correlated (IGHD, IGHM secreted, IGKC).

In CD4 T cells (FIG. 4B), GOS2 is highly correlated with CD11c surface expression and still well correlated with CD14, CD141 and CD123 surface expression. KLRB1 gene expression is positively correlated with CD2 and CD45RO surface expression. This information is useful, because CD45RO is not encoded by a specific gene and thus not accessible in standard scRNA-Seq data. CD56 surface expression is positively correlated with NKG7 and GNLY gene expression, useful for the identification of NK T cells. A useful and strong negative correlation is found between CCL5 (RANTES) and CD126 and CD27 surface expression.

In CD8 T cells (FIG. 4C), LEF1 gene expression is well correlated with surface expression of CD126 (IL-6R), CD197 (CCR7), CD127 (IL-7R) and CD27. In fact, a whole block of other genes is also positively correlated with this same group of cell surface markers: PIK3IP1, DUSP1, FOSB, JUNB, MYC, SELL, PASK and CD48. All these markers are typically found in naive CD8 T cells. For CD8 NKT cells, surface expression of CD56 is highly correlated with GNLY gene expression.

In classical monocytes (FIG. 4D), CD2 surface expression was well correlated with FCER1A gene expression. In intermediate monocytes (FIG. 4C), a cluster of genes (S100A9, S100A12, LYZ, CD163, RNASE6, VCAN) is well correlated with the surface markers CD192 (CCR2), CD137 (4-1BB), CD142 (tissue factor), CD38, CD11b (Mac-1) and CD14. In nonclassical monocytes (FIG. 4F), a large cluster of genes (FCER1G, C5AR1, C10Orf54, IFITM3, LILRB1, FCGR3A, ANXA5, DUSP1, BCL2A1, LYN, TKT, CXCL16, FTH1, FCN1, CD83, NAMP, SIGLEC10, SLC7A7, TGFB1 and ADGRE1) is highly to moderately correlated with CD11c, CD74 (HLA-DR), CD123, and CD141 (THBD) surface expression. Another group of genes (NKG7, CCL5, CST7, GNLY) is positively correlated with CD2 and CD38 surface expression. By contrast, FGFBP2 is highly correlated with CD38, but not CD2.

In B cells (FIG. 4G), IGHA1-secreted is highly correlated with CD27. IGHG1-secreted and IGHG2-secreted expression are both are well correlated with CD27, CD11c and CD183 surface markers. A cluster of genes (S100A 12, CD14, LYZ) was well correlated with CD14 and CD36 surface expression. A second cluster of genes (FCER1G, FPR1, C5AR1, FCN1 and, to a lesser degree, S100A9 correlated with CD14, CD36 and CD141 surface expression.

In NK cells (FIG. 4H), SELL and GZMK were well correlated with CD56 surface expression, providing that they are more expressed in immature (CD16−) NK cells.

### Example 6: Results

sc-RNA-Seq with CITE-Seq was used to identify 50 clusters of PBMCs in 31 participants of the WIHS study. The most diversity was in CD8 T cells (14 clusters) and monocytes (14 clusters: 6 classical, 4 intermediate and 4 nonclassical). CD4 T cells were resolved into 11 clusters, B cells into 7 clusters and NK cells into 4 clusters. Importantly, 8 of these clusters showed significant differences when comparing the cell abundance among the 4 types of participants. The most exciting subset is the intermediate monocyte cluster 2, which was highly significantly (p<0.0001) reduced in participants with CVD. Other subsets that changed cell proportions with CVD were the intermediate monocyte cluster 4 and the B cell cluster 1, which is not affected by cholesterol control. In contrast, the CD4 cluster 9 is highly expanded by CVD and its proportion is corrected by cholesterol control.

In peripheral blood, CD4 T cells counts are significantly lower in people living with HIV. HIV directly infects CD4 T cells and causes their numbers to drop, a hallmark of HIV disease.⁷⁸ CD8 cluster 3 (CCR5+ EM CD8 T) is high in HIV+CVD+ participants that receive cholesterol-controlling medication. It is possible that CD8 cluster 3 may drive the previously reported association between CD8 T cell numbers and cardiovascular disease risk.^{79,80} The number of cells in CD8 cluster 5 (CD8 NKT cells), is significantly decreased in patients with HIV. It has been shown that individuals with HIV present a considerable depletion of NKT cells81 with poor functionality.^{82,83}

INT monocytes cluster 2 and cluster 4 (which is positive for tissue factor) are high in HIV compared to HIV+CVD+. The intermediate monocytes in cluster 2 seem to rebound under cholesterol control, to levels similar to HIV+CVD− participants. Elevated circulating leukocytes are associated with increased CVD risk, with this association primarily driven by monocytes and neutrophils (not contained in PBMCs). Monocyte abundance is an independent risk factor for CVD, with monocytosis causally linked to both the acceleration of atherosclerotic lesion progression and impaired lesion regression.⁸⁴ In the Apoe−/− mouse model of atherosclerosis, combined inhibition of CCL2, CX3CR1 and CCR5 results in low monocytes and a 90% reduction in atherosclerotic lesion burden.85 Among monocytes, classical (CM, CD14+CD16−) and intermediate (INT, CD14+CD16+) monocytes are considered pro-atherogenic,⁵² whereas nonclassical monocytes (NCM, CD14−CD16+) have been shown to dampen atherosclerosis in animal models.^{54,86}

B cell clusters 1 and 6 show opposite patterns. It is not known how these B cell subsets are related to atherosclerosis. The proportion of cells in cluster 1 (likely mixed pre-plasmablasts and naive) was significantly greater in HIV+ patients compared with healthy subjects, results consistent with prior studies.^{32,33} Cluster 6 (activated memory) was highly significantly reduced in number in HIV+ subjects. Although prior studies have demonstrated that HIV infection leads to an increase in number of activated memory B cells, antiretroviral therapy (ART) can reverse the phenomenon and decreases number of activated B cells significantly.⁸⁷

Most HIV+ patients in this study were on ART. In addition to these significant changes, there was a trend for an increase (p=0.053) in cluster 5 (CD11c+ B cells) in HIV+ compared to healthy participants, which is consistent with findings from prior studies that CD11c+ pathologic B cells are prevalent in HIV patients.³⁴

NK cluster 1 cells are more abundant in HIV+CVD+ participants compared to healthy participants. CD56bright NK cells accumulate in human atherosclerotic lesions, possibly contributing to plaque instability.⁸⁹

Example 7: The present invention includes methods to determine a risk of a cardiovascular disease (CVD) or a subclinical CVD (sCVD), the likelihood of CVD or sCVD, diagnosing CVD or sCVD, or treating CVD or sCVD in a subject, by detecting a subset of intermediate monocyte cell populations in the immune cells, wherein a percentage of the subsets as compared to a total intermediate monocyte cell population present in the isolated immune cells is determined; comparing a proportion of the subsets subset of intermediate monocyte cell populations determined with a statistical sample representative of a proportion of equivalent subsets in a total intermediate monocyte cell populations from a subject that does not have a cardiovascular disease; and determining that the subject has an increased risk for cardiovascular disease where the subject has a decrease in the certain subsets, has an increase in another subset, or both, as compared to the statistical sample.

Gating of intermediate monocyte (INT) subpopulations: INT are gated as CD3−CD19−CD14+CD16+CD56−. The defining markers for each INT subpopulation is provided as follows:

INT2 some IL6RA, some CD137, CD141 positive, CD142 positive, CD163 positive, CCR2 positive, some even high, most are CCR4+, CCR5+, few express CD206, all CD36+ and CD38+, almost all CD45RO+, all CD69+, CD86+CD9+

INT3 similar to INT2, but some express CD154, all CCR2+, all CCR4+, most CD206+, most LAG3+, some express CD4, all CD69+

INT4 is low in IL6RA, lower in CD137 than INT 2 and 3, does not express CD154, CD163 low, CCR2 low, CCR4 low, C CR5 low, CD206 low, LAG3 negative, CD36 lower than INT2 and 3, CD38 low, express some CD45RA, CD9+.

In one embodiment, the present invention provides a method of gating an intermediate monocyte population to find these cells and measure their proportions. In such an embodiment, the “biomarker” for determining whether a patient has, it as risk of developing, or should be treated for CVD or subclinical CVD is the change in proportions of intermediate monocyte subsets between the subject and a healthy patient or statistical sample from a healthy patient.

Example 8: The present invention includes methods to determine a risk of a cardiovascular disease (CVD) or a subclinical CVD (sCVD), the likelihood of CVD or sCVD, diagnosing CVD or sCVD, or treating CVD or sCVD in a subject, by detecting a subset biomarkers in one or more immune cells of the subject, wherein the expression or activity of said biomarkers as compared to a statistical sample representative of a proportion of equivalent biomarkers in the immune cells from a subject that does not have a cardiovascular disease; and determining that the subject has an increased risk for cardiovascular disease where the subject has an increase in the expression or activity of one or more biomarkers as compared to the statistical sample. The biomarkers and their related immune cell subsets are set forth on Tables A to G, herein.

Tables A to G. Significantly differentially expressed genes for each cell type. Differentially expressed genes are from one cluster compared against the rest in the same cell type. gene: gene name, p_val: raw p value, avg_logFC: average log 2 fold change, pct.1: percent of cells in hat cluster that express this gene, pct.2: percent of cells in all other clusters that express this gene, p_val_adj: p value adjusted by Benjamini-Hochberg for multiple comparisons, cluster: cluster number as noted in UMAP. (A) CD4 T cells, (B) CD8 T cells, (C) Classical monocytes, (D) Intermediate monocytes, (E) Nonclassical monocytes, (F) Nonclassical monocytes, (G) B cells, (F) NK cells.

(A) CD4 T cells

| gene | p_val | avg_logFC | pct.1 | pct.2 | p_val_adj | cluster |
|---|---|---|---|---|---|---|
| LEF1 | 0 | 2.0329031 | 0.513 | 0.082 | 0 | 2 |
| CCR7 | 0 | 1.8002173 | 0.341 | 0.051 | 0 | 2 |
| PASK | 1.70E−261 | 1.7203148 | 0.342 | 0.073 | 8.23E−259 | 2 |
| MYC | 6.88E−221 | 1.5423473 | 0.343 | 0.086 | 3.34E−218 | 2 |
| SELL | 3.42E−243 | 1.4967887 | 0.399 | 0.109 | 1.66E−240 | 2 |
| PIK3IP1 | 0 | 1.4927414 | 0.761 | 0.347 | 0 | 2 |
| TXK | 1.53E−138 | 1.3697898 | 0.137 | 0.019 | 7.43E−136 | 2 |
| CD27 | 2.09E−133 | 1.3251843 | 0.267 | 0.08 | 1.01E−130 | 2 |
| IL7R | 1.57E−185 | 1.2455184 | 0.427 | 0.154 | 7.60E−183 | 2 |
| FOSB | 7.52E−163 | 1.1060644 | 0.675 | 0.434 | 3.65E−160 | 2 |
| JUNB | 1.55E−149 | 1.0762996 | 0.61 | 0.361 | 7.52E−147 | 2 |
| GNLY | 5.77E−237 | 1.4319902 | 0.895 | 0.409 | 2.80E−234 | 5 |
| KLRF1 | 9.47E−48 | 1.05414 | 0.143 | 0.036 | 4.59E−45 | 5 |
| S100A9 | 1.21E−07 | 1.3499789 | 0.112 | 0.068 | 5.85E−05 | 6 |
| MYC | 9.92E−78 | 1.2199142 | 0.31 | 0.108 | 4.81E−75 | 7 |
| IL4R | 1.29E−53 | 1.0908685 | 0.215 | 0.074 | 6.25E−51 | 7 |
| ITGAX | 2.36E−123 | 1.8119865 | 0.313 | 0.028 | 1.14E−120 | 8 |
| TRDC | 1.12E−106 | 1.7834228 | 0.247 | 0.02 | 5.45E−104 | 8 |
| KLRC3 | 5.57E−48 | 1.3154248 | 0.256 | 0.045 | 2.70E−45 | 8 |
| FCGR3A | 1.13E−38 | 1.2871792 | 0.374 | 0.103 | 5.50E−36 | 8 |
| HOPX | 1.97E−25 | 1.0842147 | 0.502 | 0.222 | 9.55E−23 | 8 |
| LYN | 1.44E−26 | 1.0442727 | 0.181 | 0.038 | 7.00E−24 | 8 |
| GZMK | 4.68E−27 | 1.2761059 | 0.372 | 0.141 | 2.27E−24 | 10 |
| CCR7 | 1.84E−51 | 1.1932127 | 0.253 | 0.083 | 8.91E−49 | 12 |
| LEF1 | 2.27E−72 | 1.0909454 | 0.373 | 0.13 | 1.10E−69 | 12 |
| PASK | 1.16E−45 | 1.0581898 | 0.274 | 0.102 | 5.62E−43 | 12 |
| SELL | 1.87E−51 | 1.0235325 | 0.344 | 0.139 | 9.08E−49 | 12 |
| TRDC | 1.15E−35 | 1.6849589 | 0.117 | 0.021 | 5.56E−33 | 14 |
| FCGR3A | 6.24E−80 | 1.4403569 | 0.388 | 0.098 | 3.03E−77 | 14 |
| GZMB | 1.31E−89 | 1.2254516 | 0.6 | 0.196 | 6.34E−87 | 14 |
| KLRC3 | 1.25E−30 | 1.1715095 | 0.168 | 0.045 | 6.07E−28 | 14 |
| KLRC1 | 1.75E−41 | 1.1458533 | 0.154 | 0.03 | 8.47E−39 | 14 |
| KLRB1 | 4.88E−45 | 1.1248609 | 0.383 | 0.14 | 2.37E−42 | 14 |
| GNLY | 7.98E−101 | 1.0651549 | 0.902 | 0.424 | 3.87E−98 | 14 |

(B) CD8 T cells

| gene | p_val | avg_logFC | pct.1 | pct.2 | p_val_adj | cluster |
|---|---|---|---|---|---|---|
| LEF1 | 0 | 2.0329031 | 0.513 | 0.082 | 0 | 2 |
| CCR7 | 0 | 1.8002173 | 0.341 | 0.051 | 0 | 2 |
| PASK | 1.70E−261 | 1.7203148 | 0.342 | 0.073 | 8.23E−259 | 2 |
| MYC | 6.88E−221 | 1.5423473 | 0.343 | 0.086 | 3.34E−218 | 2 |
| SELL | 3.42E−243 | 1.4967887 | 0.399 | 0.109 | 1.66E−240 | 2 |
| PIK3IP1 | 0 | 1.4927414 | 0.761 | 0.347 | 0 | 2 |
| TXK | 1.53E−138 | 1.3697898 | 0.137 | 0.019 | 7.43E−136 | 2 |
| CD27 | 2.09E−133 | 1.3251843 | 0.267 | 0.08 | 1.01E−130 | 2 |
| IL7R | 1.57E−185 | 1.2455184 | 0.427 | 0.154 | 7.60E−183 | 2 |
| FOSB | 7.52E−163 | 1.1060644 | 0.675 | 0.434 | 3.65E−160 | 2 |
| JUNB | 1.55E−149 | 1.0762996 | 0.61 | 0.361 | 7.52E−147 | 2 |
| GNLY | 5.77E−237 | 1.4319902 | 0.895 | 0.409 | 2.80E−234 | 5 |
| KLRF1 | 9.47E−48 | 1.05414 | 0.143 | 0.036 | 4.59E−45 | 5 |
| S100A9 | 1.21E−07 | 1.3499789 | 0.112 | 0.068 | 5.85E−05 | 6 |
| MYC | 9.92E−78 | 1.2199142 | 0.31 | 0.108 | 4.81E−75 | 7 |
| IL4R | 1.29E−53 | 1.0908685 | 0.215 | 0.074 | 6.25E−51 | 7 |
| ITGAX | 2.36E−123 | 1.8119865 | 0.313 | 0.028 | 1.14E−120 | 8 |
| TRDC | 1.12E−106 | 1.7834228 | 0.247 | 0.02 | 5.45E−104 | 8 |
| KLRC3 | 5.57E−48 | 1.3154248 | 0.256 | 0.045 | 2.70E−45 | 8 |
| FCGR3A | 1.13E−38 | 1.2871792 | 0.374 | 0.103 | 5.50E−36 | 8 |
| HOPX | 1.97E−25 | 1.0842147 | 0.502 | 0.222 | 9.55E−23 | 8 |
| LYN | 1.44E−26 | 1.0442727 | 0.181 | 0.038 | 7.00E−24 | 8 |
| GZMK | 4.68E−27 | 1.2761059 | 0.372 | 0.141 | 2.27E−24 | 10 |
| CCR7 | 1.84E−51 | 1.1932127 | 0.253 | 0.083 | 8.91E−49 | 12 |
| LEF1 | 2.27E−72 | 1.0909454 | 0.373 | 0.13 | 1.10E−69 | 12 |
| PASK | 1.16E−45 | 1.0581898 | 0.274 | 0.102 | 5.62E−43 | 12 |
| SELL | 1.87E−51 | 1.0235325 | 0.344 | 0.139 | 9.08E−49 | 12 |
| TRDC | 1.15E−35 | 1.6849589 | 0.117 | 0.021 | 5.56E−33 | 14 |
| FCGR3A | 6.24E−80 | 1.4403569 | 0.388 | 0.098 | 3.03E−77 | 14 |
| GZMB | 1.31E−89 | 1.2254516 | 0.6 | 0.196 | 6.34E−87 | 14 |
| KLRC3 | 1.25E−30 | 1.1715095 | 0.168 | 0.045 | 6.07E−28 | 14 |
| KLRC1 | 1.75E−41 | 1.1458533 | 0.154 | 0.03 | 8.47E−39 | 14 |
| KLRB1 | 4.88E−45 | 1.1248609 | 0.383 | 0.14 | 2.37E−42 | 14 |
| GNLY | 7.98E−101 | 1.0651549 | 0.902 | 0.424 | 3.87E−98 | 14 |

(C) Classical monocytes

| gene | p_val | avg_logFC | pct.1 | pct.2 | p_val_adj | cluster |
|---|---|---|---|---|---|---|
| FCER1A | 1.93E−230 | 2.94537682 | 0.368 | 0.011 | 9.38E−228 | 6 |
| CLEC10A | 6.74E−87 | 2.40551253 | 0.472 | 0.08 | 3.27E−84 | 6 |
| CD1C | 2.32E−48 | 1.81098677 | 0.25 | 0.038 | 1.12E−45 | 6 |
| HLA.DRA | 4.01E−45 | 1.32941853 | 0.906 | 0.789 | 1.95E−42 | 6 |
| HLA.DQB1 | 3.31E−22 | 1.2525502 | 0.67 | 0.426 | 1.61E−19 | 6 |
| HLA.DPA1 | 2.17E−32 | 1.25169794 | 0.764 | 0.516 | 1.05E−29 | 6 |
| RGS1 | 2.01E−21 | 1.0365779 | 0.481 | 0.214 | 9.74E−19 | 6 |
| CD74 | 2.64E−48 | 1.00823372 | 0.981 | 0.913 | 1.28E−45 | 6 |

(D) Intermediate monocytes

| gene | p_val | avg_logFC | pct.1 | pct.2 | p_val_adj | cluster |
|---|---|---|---|---|---|---|
| THBS1 | 2.83E−06 | 1.08098 | 0.271 | 0.153 | 0.00137 | 2 |

(E) Nonclassical monocytes

| gene | p_val | avg_logFC | pct.1 | pct.2 | p_val_adj | cluster |
|---|---|---|---|---|---|---|
| VMO1 | 5.33E−54 | 2.54831 | 0.402 | 0.04 | 2.58E−51 | 1 |
| IL1B | 1.28E−15 | 2.00858 | 0.211 | 0.055 | 6.21E−13 | 1 |
| CXCL16 | 2.69E−65 | 1.77535 | 0.615 | 0.122 | 1.31E−62 | 1 |
| C5AR1 | 1.80E−89 | 1.67038 | 0.772 | 0.148 | 8.74E−87 | 1 |
| C10orf54 | 3.54E−73 | 1.60862 | 0.789 | 0.246 | 1.72E−70 | 1 |
| FCER1G | 4.73E−92 | 1.56885 | 0.882 | 0.243 | 2.29E−89 | 1 |
| BCL2A1 | 1.47E−60 | 1.55039 | 0.632 | 0.152 | 7.15E−58 | 1 |
| NAMPT | 6.08E−52 | 1.54449 | 0.584 | 0.154 | 2.95E−49 | 1 |
| TNFSF10 | 1.51E−21 | 1.52514 | 0.278 | 0.068 | 7.34E−19 | 1 |
| FTH1 | 2.63E−63 | 1.49438 | 0.64 | 0.137 | 1.27E−60 | 1 |
| TKT | 1.02E−62 | 1.46395 | 0.683 | 0.181 | 4.95E−60 | 1 |
| IFITM3 | 3.06E−73 | 1.43648 | 0.902 | 0.459 | 1.49E−70 | 1 |
| CD4 | 1.89E−31 | 1.41956 | 0.334 | 0.065 | 9.19E−29 | 1 |
| ADGRE1 | 5.40E−26 | 1.40699 | 0.287 | 0.059 | 2.62E−23 | 1 |
| SIGLEC10 | 2.18E−49 | 1.38855 | 0.525 | 0.109 | 1.06E−46 | 1 |
| HLA.DRA | 5.68E−60 | 1.34353 | 0.817 | 0.327 | 2.75E−57 | 1 |
| CD86 | 1.06E−30 | 1.30777 | 0.32 | 0.061 | 5.15E−28 | 1 |
| IL3RA | 3.77E−25 | 1.2905 | 0.228 | 0.032 | 1.83E−22 | 1 |
| HLA.DPA1 | 2.34E−48 | 1.2877 | 0.812 | 0.423 | 1.14E−45 | 1 |
| FCGR3A | 3.79E−50 | 1.25738 | 0.885 | 0.589 | 1.84E−47 | 1 |
| GZMH | 1.05E−58 | 1.58999 | 0.566 | 0.11 | 5.09E−56 | 2 |
| TRDC | 4.25E−61 | 1.52704 | 0.59 | 0.11 | 2.06E−58 | 2 |
| NKG7 | 8.29E−111 | 1.47187 | 0.988 | 0.348 | 4.02E−108 | 2 |
| FGFBP2 | 2.74E−65 | 1.44339 | 0.669 | 0.15 | 1.33E−62 | 2 |
| IFNG | 2.01E−28 | 1.44164 | 0.301 | 0.056 | 9.73E−26 | 2 |
| GNLY | 1.34E−91 | 1.38708 | 0.937 | 0.278 | 6.50E−89 | 2 |
| LCK | 5.16E−25 | 1.349 | 0.294 | 0.063 | 2.50E−22 | 2 |
| CTSW | 3.80E−56 | 1.34552 | 0.636 | 0.155 | 1.84E−53 | 2 |
| HOPX | 1.45E−42 | 1.2465 | 0.499 | 0.113 | 7.04E−40 | 2 |
| S1PR5 | 4.81E−22 | 1.22691 | 0.268 | 0.059 | 2.34E−19 | 2 |
| CD3E | 7.70E−24 | 1.22206 | 0.291 | 0.065 | 3.74E−21 | 2 |
| CST7 | 1.52E−70 | 1.18106 | 0.823 | 0.224 | 7.37E−68 | 2 |
| TRBC2 | 5.79E−38 | 1.13973 | 0.545 | 0.166 | 2.81E−35 | 2 |
| KLRK1 | 2.35E−41 | 1.13969 | 0.452 | 0.09 | 1.14E−38 | 2 |
| CD2 | 3.83E−24 | 1.13957 | 0.261 | 0.047 | 1.86E−21 | 2 |
| CD247 | 3.49E−38 | 1.12918 | 0.485 | 0.119 | 1.69E−35 | 2 |
| IL2RB | 4.85E−36 | 1.11253 | 0.445 | 0.103 | 2.35E−33 | 2 |
| IL32 | 5.49E−67 | 1.10702 | 0.776 | 0.206 | 2.66E−64 | 2 |
| SYNE1 | 8.27E−18 | 1.09947 | 0.219 | 0.05 | 4.01E−15 | 2 |
| CCL5 | 1.40E−86 | 1.05872 | 0.958 | 0.299 | 6.79E−84 | 2 |
| KLRB1 | 3.03E−10 | 1.07071 | 0.415 | 0.219 | 1.47E−07 | 3 |
| C1QB | 2.23E−05 | 1.61387 | 0.127 | 0.039 | 0.010813 | 4 |
| FCN1 | 1.34E−31 | 1.47473 | 0.72 | 0.238 | 6.49E−29 | 4 |
| NR4A1 | 2.45E−10 | 1.3837 | 0.441 | 0.181 | 1.19E−07 | 4 |
| C1QA | 8.54E−10 | 1.31751 | 0.22 | 0.062 | 4.14E−07 | 4 |
| TNF | 2.10E−05 | 1.28729 | 0.297 | 0.146 | 0.010195 | 4 |
| LILRB1 | 6.42E−32 | 1.20804 | 0.78 | 0.267 | 3.11E−29 | 4 |
| S100A9 | 5.47E−19 | 1.11651 | 0.602 | 0.215 | 2.65E−16 | 4 |
| ASAH1 | 2.69E−12 | 1.07052 | 0.475 | 0.19 | 1.30E−09 | 4 |
| SLC7A7 | 7.26E−20 | 1.05582 | 0.432 | 0.11 | 3.52E−17 | 4 |

(F) B cells

| gene | p_val | avg_logFC | pct.1 | pct.2 | p_val_adj | cluster |
|---|---|---|---|---|---|---|
| IGHG1-secreted | 8.73E−61 | 1.51618418 | 0.636 | 0.186 | 4.24E−58 | 2 |
| IGHG2-secreted | 1.19E−56 | 1.28478718 | 0.701 | 0.237 | 5.79E−54 | 2 |
| CD27 | 3.69E−26 | 1.15671581 | 0.169 | 0.03 | 1.79E−23 | 2 |
| S100A10 | 7.58E−38 | 1.10672805 | 0.548 | 0.209 | 3.68E−35 | 2 |
| IGHG4-secreted | 1.37E−20 | 1.09287357 | 0.107 | 0.015 | 6.62E−18 | 2 |
| LGALS1 | 3.12E−15 | 1.46625862 | 0.418 | 0.195 | 1.52E−12 | 5 |
| ITGAX | 9.33E−38 | 1.3608377 | 0.254 | 0.037 | 4.53E−35 | 5 |
| CST7 | 1.87E−14 | 1.23481632 | 0.113 | 0.02 | 9.06E−12 | 5 |
| IGHG2-secreted | 1.09E−51 | 1.15766919 | 0.74 | 0.249 | 5.31E−49 | 5 |
| IGHG1-secreted | 1.15E−47 | 1.10469771 | 0.65 | 0.199 | 5.56E−45 | 5 |
| RGS1 | 1.19E−14 | 1.09977091 | 0.458 | 0.228 | 5.79E−12 | 5 |
| NKG7 | 5.54E−12 | 1.00166839 | 0.169 | 0.048 | 2.69E−09 | 5 |
| S100A9 | 2.28E−36 | 3.87625476 | 0.692 | 0.069 | 1.11E−33 | 7 |
| FCN1 | 1.51E−84 | 3.7410929 | 0.846 | 0.041 | 7.32E−82 | 7 |
| FCGR3A | 2.04E−46 | 3.58040718 | 0.385 | 0.015 | 9.90E−44 | 7 |
| CXCL8 | 1.63E−24 | 3.56990364 | 0.5 | 0.052 | 7.91E−22 | 7 |
| CCL3 | 5.08E−31 | 3.45008068 | 0.462 | 0.034 | 2.47E−28 | 7 |
| IL1B | 2.50E−21 | 3.28631602 | 0.346 | 0.028 | 1.21E−18 | 7 |
| LYZ | 4.22E−60 | 3.05706018 | 0.538 | 0.022 | 2.05E−57 | 7 |
| FCER1G | 3.03E−89 | 3.01443852 | 0.731 | 0.027 | 1.47E−86 | 7 |
| IFITM3 | 2.05E−18 | 3.00967893 | 0.692 | 0.138 | 9.94E−16 | 7 |
| CD14 | 1.12E−76 | 2.93965149 | 0.423 | 0.009 | 5.45E−74 | 7 |
| C5AR1 | 4.10E−76 | 2.91219048 | 0.5 | 0.014 | 1.99E−73 | 7 |
| CXCL2 | 3.60E−18 | 2.90409027 | 0.308 | 0.026 | 1.75E−15 | 7 |
| SELPLG | 3.61E−53 | 2.76283159 | 0.462 | 0.018 | 1.75E−50 | 7 |
| LGALS1 | 4.23E−18 | 2.66061676 | 0.808 | 0.203 | 2.05E−15 | 7 |
| NAMPT | 5.72E−16 | 2.51698383 | 0.462 | 0.066 | 2.78E−13 | 7 |
| LGALS3 | 1.86E−71 | 2.37633178 | 0.654 | 0.027 | 9.00E−69 | 7 |
| CXCL16 | 7.25E−20 | 2.31859618 | 0.385 | 0.035 | 3.52E−17 | 7 |
| S100A12 | 1.07E−32 | 2.1651655 | 0.269 | 0.01 | 5.17E−30 | 7 |
| LILRB1 | 1.43E−32 | 2.13972361 | 0.5 | 0.037 | 6.94E−30 | 7 |
| FPR1 | 2.41E−100 | 2.09784743 | 0.423 | 0.006 | 1.17E−97 | 7 |

(G) NK cells

| gene | p_val | avg_logFC | pct.1 | pct.2 | p_val_adj | cluster |
|---|---|---|---|---|---|---|
| GZMH | 2.85E−06 | 1.02055 | 0.503 | 0.341 | 0.00138 | 4 |

Methods. Study characteristics and sample selection. The Women's Interagency HIV Study (WIHS) was initiated in 1994 at six (now expanded to ten) U.S. locations.^{22,36} It is an ongoing prospective cohort study of over 4,000 women with or at risk of HIV infection. Recruitment in the WIHS occurred in four time points (1994-1995, 2001-2002, 2010-2012, and 2013-2015) from HIV primary care clinics, hospital-based programs, community outreach and support groups. Briefly, the WIHS involves semi-annual follow-up visits, during which participants undergo similar detailed examinations, specimen collection, and structured interviews assessing health behaviors, medical history, and medication use. All participants provided informed consent, and each site's Institutional Review Board approved the studies.

Participants from the current analysis were part of a vascular substudy nested within the WIHS.^{100,101} The baseline visit for the vascular substudy occurred between 2004 and 2006, and a follow-up visit occurred on average seven years later. Participants underwent high-resolution B-mode carotid artery ultrasound to image six locations in the right carotid artery: the near and far walls of the common carotid artery, carotid bifurcation, and internal carotid artery. A standardized protocol was used at all sites,^{92,102} and measurements of carotid artery focal plaque, a marker of subclinical atherosclerosis, were obtained at a centralized reading center (U. of Southern California). Subclinical CVD (sCVD) was defined based on the presence of one or more carotid artery lesions.⁹²

A two-by-two factorial design based on HIV, CVD and cholesterol treatment (e.g., statins) was used. CVD was defined as presence of carotid artery focal plaque at either vascular substudy visit to define four groups of eight participants each: 1) HIV, 2) HIV and CVD, 3) HIV and CVD treated with cholesterol-lowering drugs and 4) women without HIV or CVD (healthy). HIV infection status was ascertained by enzyme-linked immunosorbent assay (ELISA) and confirmed by Western blot. C+ participants either had one or more plaques at each vascular substudy visit, or more than one plaque at a single visit. C− participants with self-reported coronary heart disease or current lipid-lowering therapy use were excluded. The participants within the group were matched by race/ethnicity, age at the baseline vascular substudy visit (within 5 years), smoking history, and date of specimen collection (within 1 year). Demographic, clinical, and laboratory variables were assessed from the same study visit using standardized protocols. The median age at the baseline study visit was 45 years (IQR 40-51), and 96% of participants were either of black race or Hispanic ethnicity. Most (86%) reported a history of smoking. Substance use was highly prevalent, with 43% of HIV+ and 50% of HIV-participants reporting either a history of injection drug use; current use of crack, cocaine, or heroin; or alcohol use (>14 drinks per week). Among HIV+ participants, over 80% reported use of HAART at the time PBMCs were obtained, and 59% reported an undetectable HIV-1 RNA level. The median CD4+ T-cell count was 585 cells/μL (IQR 382-816) in HIV+ women without sCVD and 535 cells/μL (IQR 265-792) in HIV+ women with sCVD.

Preparation of PBMC samples for CITE-seq. On two separate days, 16 tubes of PBMCs were thawed using a 37° C. water bath and tubes filled with 8 mL of complete RPMI-1640 solution (cRPMI-1640 contains Human Serum Albumin, HEPES, Sodium pyruvate, MEM-NEAA, Penicillin-Streptomicyn, GlutaMax and Mercaptoethanol). The tubes were centrifuged at 400 g for 5 minutes and then resuspended in cold staining buffer (SB: 2% fetal bovine serum (FBS) in in phosphate-buffered saline (PBS)). Manual cell counting was performed by diluting cell concentration to the point of 100-400 cells per hemocytometer was obtained. The cells were then aliquoted to a total count of 1 million cells each and incubated on ice with Fc Block at a 1:20 dilution. These tubes were then centrifuged at 400 g for 5 minutes and then resuspended in 180 uL of SB and transferred to their respective Sample Multiplexing Kit tubes. The cells were incubated for 20 minutes at room temperature. The cells were then transferred to 5 mL polystyrene tubes and washed with 3 mL of SB then centrifuged at 400 g for 5 minutes. The addition of 2 mL of SB to the tubes and centrifugation was repeated 2 more times for a total of 3 washes. The cells were resuspended in 400 uL of SB and a 2 uL of DRAQ7 and Calcein AM were added to each tube. The viability and cell count of each tube was determined using the BD Rhapsody Scanner (Scanner). Tubes were then pooled in equal proportions with total cell counts not to exceed 1 million cells. The tubes were then centrifuged at 400 g for 5 minutes and resuspended in a cocktail of 40 AbSeq antibodies (2 uL each and 20 uL of SB) on ice for 30-60 minutes per manufacturer's recommendations. The tubes were then washed with 2 mL of SB followed by centrifugation at 400 g for 5 minutes. This was repeated two more times for a total of 3 washes. The cells were then counted again using the Scanner.

Library preparation and pre-sequencing quality control (QC). Once the cells were within the ideal loading concentration of ˜800 cells/uL but no more than 1000 cells/uL, the primed plate was loaded. The plate was primed and then loaded and unloaded per the User Guide described by BD when using a Scanner. The lysis buffer that was collected was removed by having the beads isolated with a magnet and the supernatant removed. Reverse Transcription was performed at 37° C. on a thermomixer at 1200 rpm for 20 minutes. Exonuclease I was then performed at 37° C. on a thermomixer at 1200 rpm for 30 minutes then immediately placed on a heat block at 80° C. for 20 minutes. The tube was then placed on ice followed by supernatant removal while beads were on a magnet. The beads were resuspended in Bead Resuspension solution (provide in BD kit). The tubes were then stored in 4° C. until further process could be performed. Per BD's protocol the reagents for PCR1 including the Human Immune Response Panel and a Customer panel of ˜100 genes were added to the beads and they were aliquoted into four 0.2 mL strip PCR tubes and incubated for 10 cycles according to BD's protocol for PCR1. A double size selection was performed using AMPure XPre beads at a ratio of 0.7×. The supernatant was retained and transferred to a new tube where an additional 100 uL of AMPure XP beads was added to it. The first tube was then washed twice with 500 uL of 80% ethanol while the second tube had the 440 uL of supernatant removed followed by two washes with 500 uL of 80% ethanol. The cDNA was eluted off the beads using 30 uL of BD elution buffer and then transferred to a 1.5 mL tube. The first tube contained the mRNA and the second contained the Sample Tags and AbSeq Libraries.

A QC/quantification check was performed on the tube containing AbSeq and Sample Tags using Agilent TapeStation high sensitivity D1000 screentape. 5 uL from each tube (mRNA and Ab/ST) was then added to their respective tubes containing the reagents for PCR2. mRNA had the reagents required for amplifying the Human Immune Response Panel and the Custom panel, while the Sample Tags had the reagents required for amplifying the Sample Tags. Each tube had 12 cycles of PCR performed according to BD′ User Guide. Each tube was then cleaned with AMPure XP beads with the following ratios 0.8× for mRNA and 1.2× for ST. Two 200 uL washes were performed during the clean-up using 80% ethanol per sample. The cDNA was eluted off using BD elution buffer.

A QC/quantification check was performed using Agilent TapeStation high sensitivity D1000 screen tape and Qubit double stranded high sensitivity DNA test kit. The mRNA was then diluted, if necessary, to a concentration of 1.2-2.7 ng/uL and the Ab/ST tube as well as the Sample Tag library from PCR2 were diluted, if necessary, to a concentration of 0.5-1.1 ng/uL. From each sample 3 uL was added to a volume of 47 uL of reagents for PCR3 as described by BD's User Guide and followed the protocol and number of cycles listed, except for AbSeq, which had 9 cycles of PCR performed as described by previous optimization. The three libraries were then cleaned with AMPure XP beads at the following ratios: mRNA 0.7× AbSeq and Sample Tag 0.8×. These samples were washed twice with 200 uL of 80% ethanol. The cDNA was eluted off the beads using BD's elution buffer. Then a final QC and quantification check was performed using the aforementioned TapeStation and Qubit kits and reagents.

Sequencing. The samples were pooled according and sequenced to the following nominal depth recommended by BD: AbSeq: n×1000 reads per cell, where n is the plexity of AbSeq used; mRNA: 20,000 reads per cell; Sample Tags: 600 reads per cell. A total of 60600 reads per cell was required for sequencing on the NovaSeq. The samples and specifications for pooling and sequencing depth, along with number of cells loaded onto each plate was optimized for S1 and S2 100 cycle kits (Illumina) with the configuration of 67×8×50 bp. Once sequencing was complete, a FASTA file was generated by BD as a reference for the AbSeq and Genes targeted with these assays. The FASTA file and FASTQ files generated by the NovaSeq were uploaded to Seven Bridged Genomics pipeline, where the data was filter in matrices and csv files. This analysis generated draft transcriptomes and surface phenotypes of 54,078 cells (496 genes, 40 antibodies). 11 genes were not expressed, i.e. had exactly 0 total reads in all cells combined. These genes were removed, leaving 485 genes for analysis.

Doublet Removal. Based on the 4 sample tags used per plate, 8,359 doublets were removed. The remaining 45,719 cells were analyzed using the Doublet Finder package on R (https://github.com/chris-mcginnis-ucsf/DoubletFinder) with the default doublet formation rate (7.5%). This removed another 3,322 doublets, leaving 42,397 Cells. Finally, all cells that had less than 128 (27) antibody molecules sequenced were removed. This removed 786 noisy cells, resulting in 41,611 cell transcriptomes. All antibody data were CLR (centered log-ratio) normalized and converted to log 2 scale.

Identifying Major Cell Types. To identify the major known cell types, antibodies to CD3, CD19, CD4, CD8, CD14, CD16 and CD56 were used. Cell type definitions:
B cells: CD19+ and CD3−
T cells: CD19− and CD3+ as T cells. (To find CD4T and CD8T further ahead)
CD4 T cells: CD4+ and CD8− T cells
CD 8 T cells: CD8+ and CD4− T cells
Monocytes and NK cells from CD19−CD3− non-B non-T cells
Classical monocytes (CM): CD14+CD16− non-B non-T cells
Intermediate monocytes (INT): CD14+CD16+ non-B non-T cells
Natural killer cells (NK): CD56+ CD14− CD20− CD123− CD206− non-B non-T cells
Nonclassical monocytes (NCM): CD14−CD16+CD56− non-B non-T cells

As is standard in the NK cell field,¹⁰³ the CD16− immature NK cells were gated to a higher level of CD56. The mature NK cells were CD19−CD3−CD16+CD56+. This resulted in 2919 B cells, 11,103 CD4 T cells, 12,843 CD8 T cells, 5,145 CM, 1009 INT, 1,108 NCM and 729 NK cells.

Thresholding. Each antibody threshold was obtained by determining its expression in a known negative cell. The populations were clearly identifiable and the thresholds were set to best separate the positive populations from the noise. In CITE-Seq, non-specific background staining is mostly caused by oligonucleotide-tagged antibody being trapped in the nanowell.¹³

Clustering. Clustering was performed using UMAP (Uniform Manifold Approximation and Projection) and Louvain clustering.⁴⁵ UMAP is a manifold learning technique for dimensionality reduction. It is based on the neighborhood graphs which captures the local relationship in the data. Because of this UMAP is able to maintain local structure and also preserve global distances in the reduced dimension, i.e., the cells that are similar in the high dimension remain close-by in the 2 dimensions and the cells that different are apart in the 2 dimensions. There are a few parameters that define the dimensionality reduction when using UMAP; 1) N_neighbours: This is used to create the neighborhood graph. It controls how the UMAP balances the local and the global structure. It gives the size of the local neighborhood the algorithm looks at while trying to obtain the lower dimensional manifold. Larger values give a more global view while smaller values give more on local view. 2) n_pcs: This gives the number of principal components of the data to consider while creating the neighborhood graph. 3) min_dist: This parameter provides the minimum distance between embedded points. Smaller values result in more dense embedding while larger values result in a more spread-out embedding. 4) Spread: This parameter determines the scale at which the points are spread out. Together with min_dist, it determines the closeness of points in the cluster. The clustering parameters used were: n_neighbors=100, n_pcs=50, min_dist=1, spread=1, random state=42. Louvain resolution was set at 0.8. Subclustering of each major cell type was based on all non-negative antibodies:
B Cells->All except CD3
CD4T Cells->All except CD19, CD8
CD8T Cells->All except CD19, CD4
CM Cells->All except CD3, CD16, CD19
NCM Cells->All except CD3, CD14, CD19, CD56
INT Cells->All except CD3, CD19
All NK Cells->All except CD4, CD14, CD20, CD123, CD206

Manual Gating. Louvain clusters produced five clusters with clearly bimodal expression of at least one cell surface marker. In CD4 T cell, 3 of the initial clusters were further divided based on the expression of CD11c, CD25, CCR6 and CXCR3. CD8 T cell had four clusters that were divided based on CCR7, CD11b, CD11c and CXCR5 surface marker expression. Nonclassical monocytes presented one cluster where the expression of CD36 was clearly not homogeneous and it was further in two. In B cells, two of the clusters were split because they showed differential expression of CD25 and CD9 within each of the clusters.

Comparing Cell Proportions. To find potential biomarkers that would be practical for clinical use, cell numbers for each participant in each cluster were identified. Statistical differences in cell proportions was calculated by log odds ratio followed by ANOVA and Tukey's multiple comparison test.

Correlation Analysis. Each antibody was correlated to its corresponding gene(s) using Spearman rank correlation and significance (R package). For each combination of gene-antibody, cells that had values below the corresponding threshold for that antibody, as well as cells with zero counts for that gene, were discarded. After this filter, any gene-antibody combination that had 10 cells or less was deemed insignificant. Finally, all non-significant (p-value>0.05) were designated a nominal value of zero as the Spearman rank correlation coefficient and only those genes or antibodies that had at least one correlation whose coefficient >=0.25 or whose coefficient <=−0.25 were selected. To uncover genes that could act as surrogates for antibody expression, all antibodies were correlated to all genes and applied the same aforementioned filtration strategy, with the exception that only those genes or antibodies that had at least one correlation whose coefficient >=0.5 or whose coefficient <=−0.5 were selected.

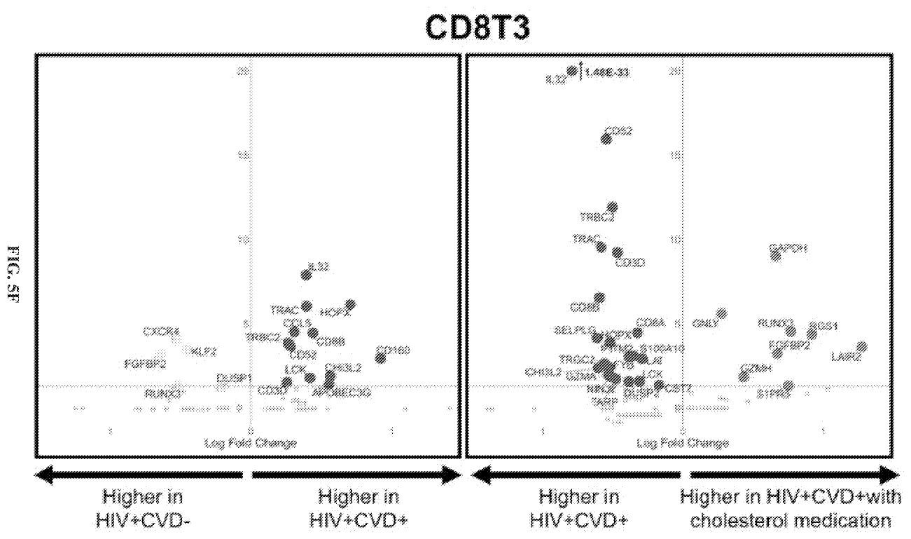

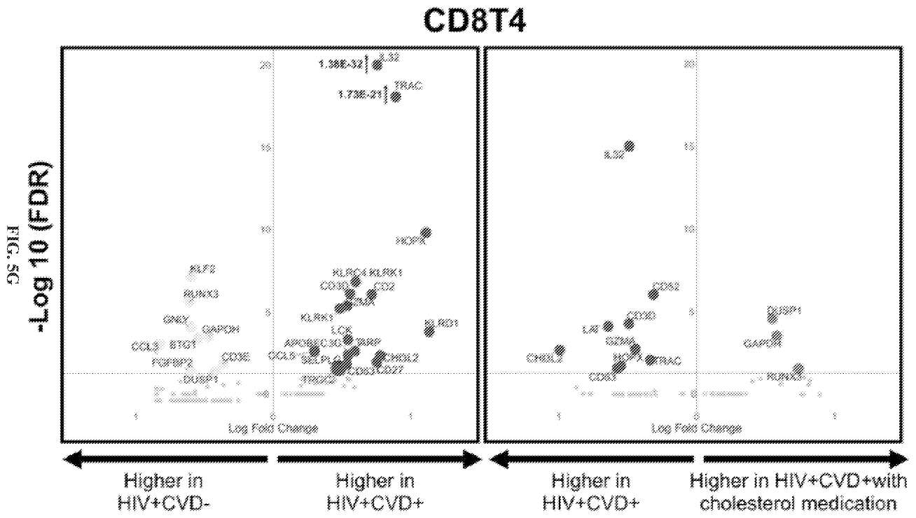

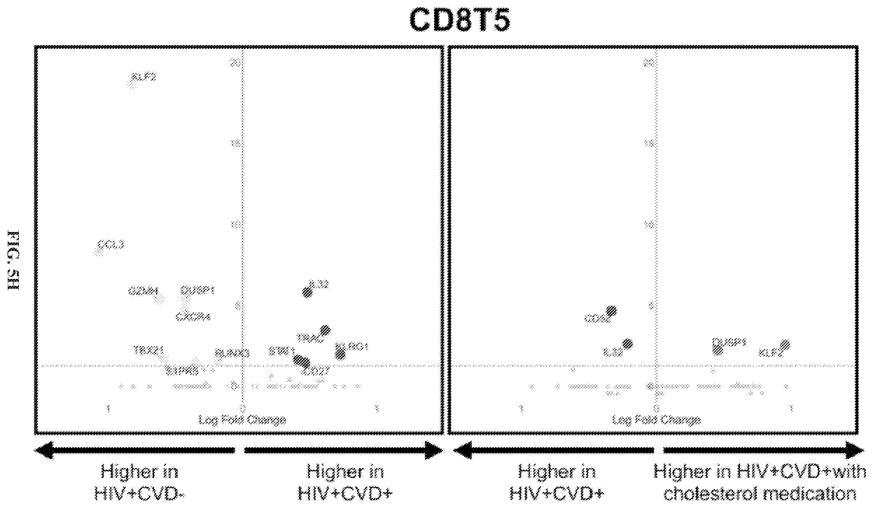

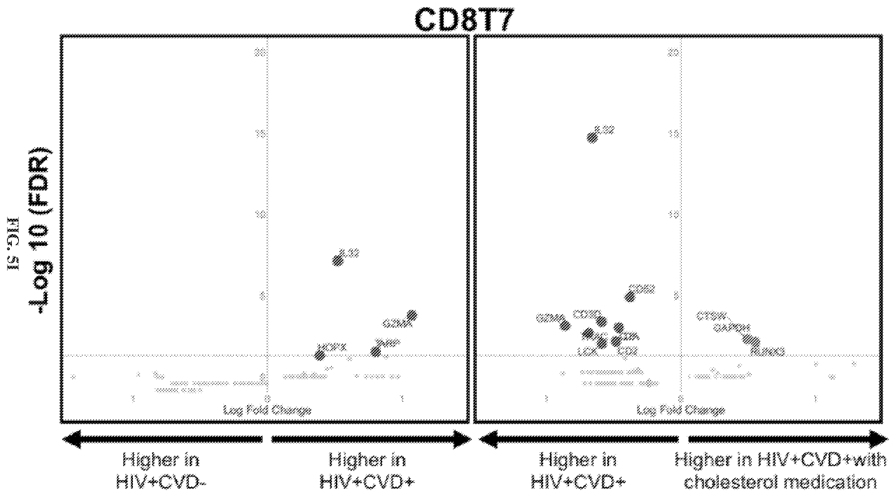

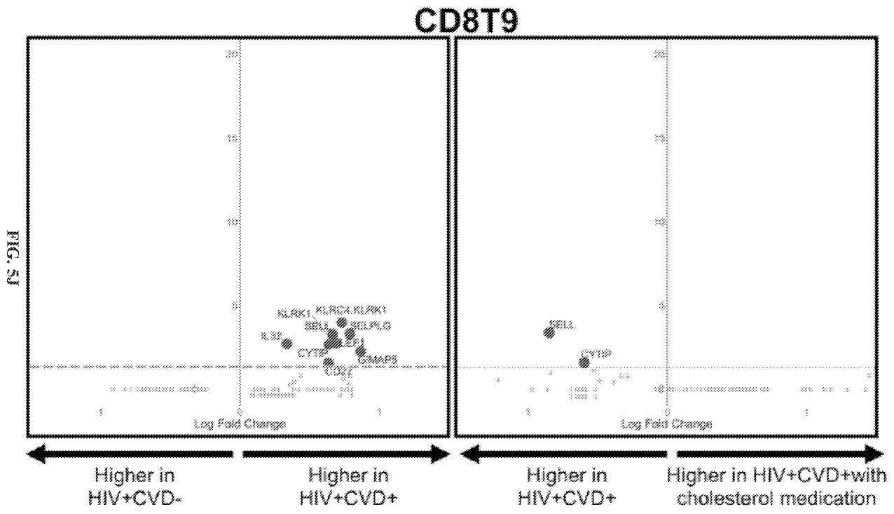

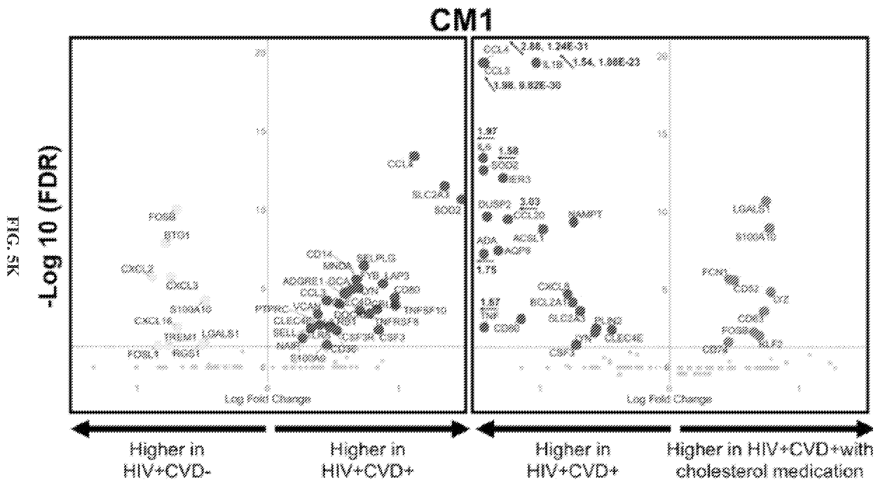

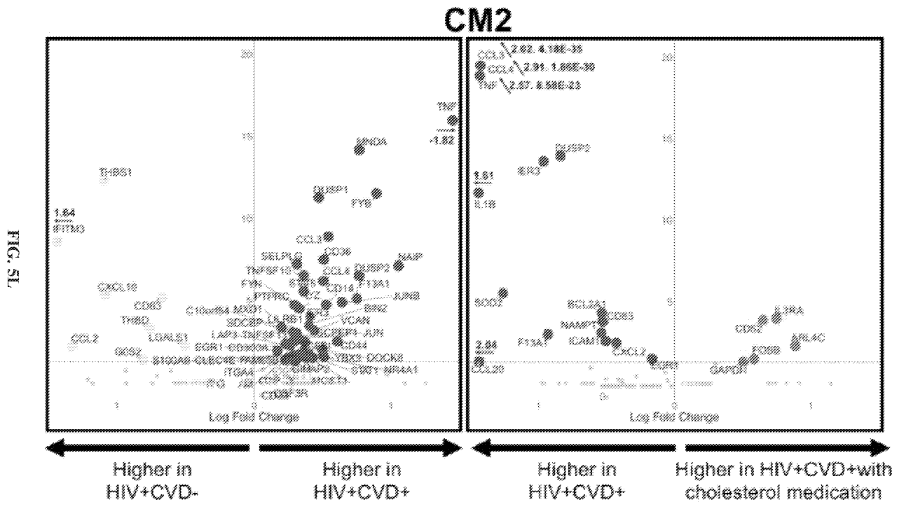

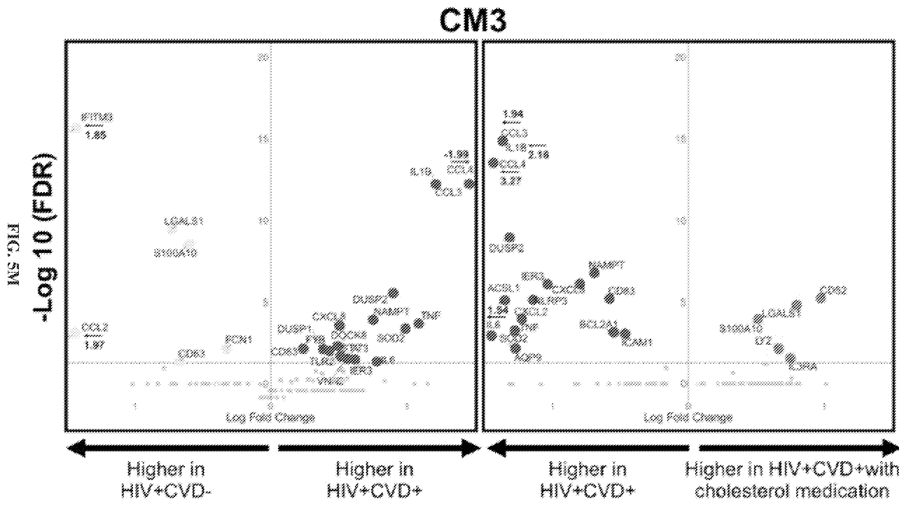

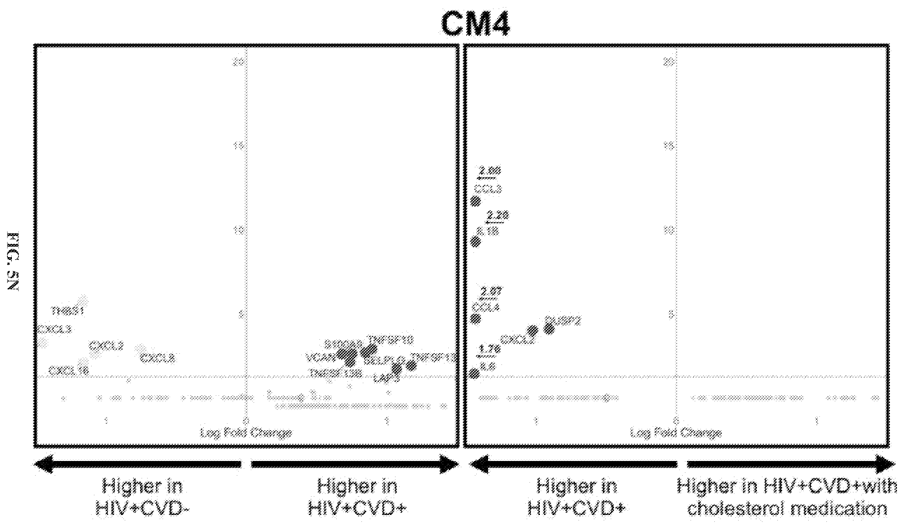

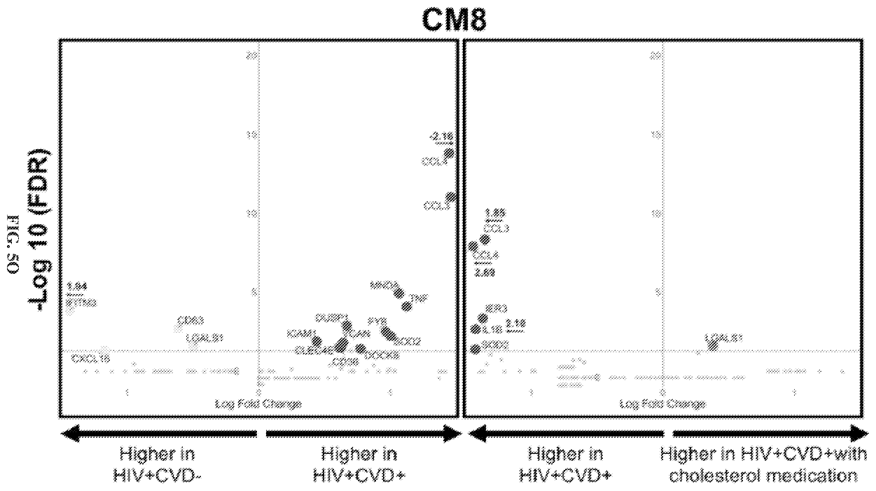

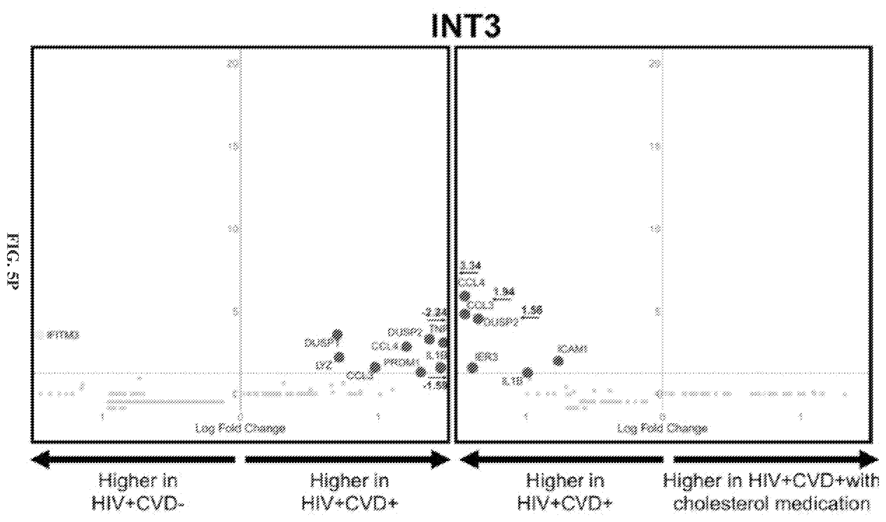

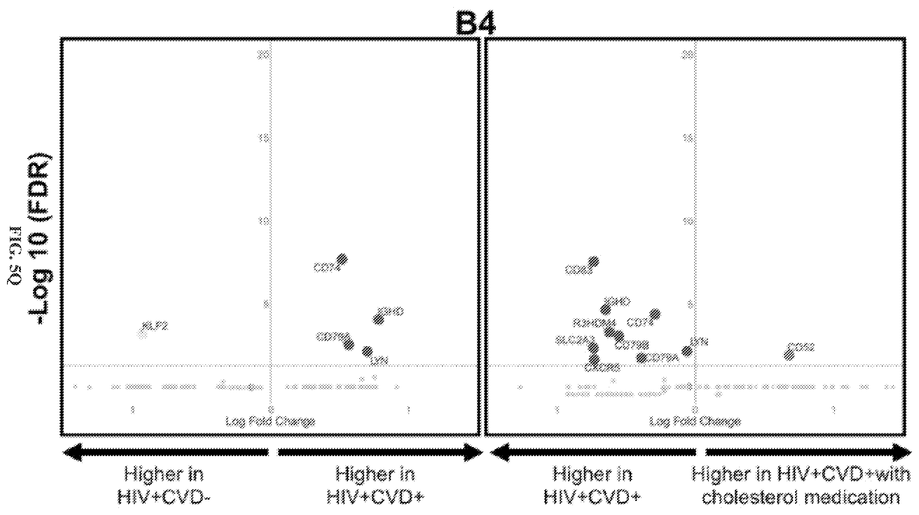

FIGS. 5A to 5Q show, Volcano plots comparing gene expression in single cells from WIHS participant types in each cluster. All 3 meaningful comparisons were calculated, but this figure is focused on HIV+CVD− vs HIV+CVD+, and HIV+CVD+ vs HIV+CVD+ with cholesterol medication; all clusters in which at least 10 genes were statistically significant. Colored dots (HIV+CVD− (left), HIV+CVD+ (center panels), and HIV+CVD+ with cholesterol medication (right) indicate 1 significantly differentiated expressed genes (FDR<0.05 and |log 2FC|>2). 3 CD4 T and 7 CD8 T cell clusters, 5 CM and 1 each INT and B cell clusters met these criteria. Dashed line indicates adjusted p-value of 0.05. It is contemplated that any embodiment discussed in this specification can be implemented with respect to any method, kit, reagent, or composition of the invention, and vice versa. Furthermore, compositions of the invention can be used to achieve methods of the invention.

It will be understood that particular embodiments described herein are shown by way of illustration and not as limitations of the invention. The principal features of this invention can be employed in various embodiments without departing from the scope of the invention. Those skilled in the art will recognize, or be able to ascertain using no more than routine experimentation, numerous equivalents to the specific procedures described herein. Such equivalents are considered to be within the scope of this invention and are covered by the claims.

All publications and patent applications mentioned in the specification are indicative of the level of skill of those skilled in the art to which this invention pertains. All publications and patent applications are herein incorporated by reference to the same extent as if each individual publication or patent application was specifically and individually indicated to be incorporated by reference.

The use of the word “a” or “an” when used in conjunction with the term “comprising” in the claims and/or the specification may mean “one,” but it is also consistent with the meaning of “one or more,” “at least one,” and “one or more than one.” The use of the term “or” in the claims is used to mean “and/or” unless explicitly indicated to refer to alternatives only or the alternatives are mutually exclusive, although the disclosure supports a definition that refers to only alternatives and “and/or.” Throughout this application, the term “about” is used to indicate that a value includes the inherent variation of error for the device, the method being employed to determine the value, or the variation that exists among the study subjects.

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps. In embodiments of any of the compositions and methods provided herein, “comprising” may be replaced with “consisting essentially of” or “consisting of”. As used herein, the phrase “consisting essentially of” requires the specified integer(s) or steps as well as those that do not materially affect the character or function of the claimed invention. As used herein, the term “consisting” is used to indicate the presence of the recited integer (e.g., a feature, an element, a characteristic, a property, a method/process step or a limitation) or group of integers (e.g., feature(s), element(s), characteristic(s), propertie(s), method/process steps or limitation(s)) only.

The term “or combinations thereof” as used herein refers to all permutations and combinations of the listed items preceding the term. For example, “A, B, C, or combinations thereof” is intended to include at least one of: A, B, C, AB, AC, BC, or ABC, and if order is important in a particular context, also BA, CA, CB, CBA, BCA, ACB, BAC, or CAB. Continuing with this example, expressly included are combinations that contain repeats of one or more item or term, such as BB, AAA, AB, BBC, AAABCCCC, CBBAAA, CABABB, and so forth. The skilled artisan will understand that typically there is no limit on the number of items or terms in any combination, unless otherwise apparent from the context.

As used herein, words of approximation such as, without limitation, “about”, “substantial” or “substantially” refers to a condition that when so modified is understood to not necessarily be absolute or perfect but would be considered close enough to those of ordinary skill in the art to warrant designating the condition as being present. The extent to which the description may vary will depend on how great a change can be instituted and still have one of ordinary skilled in the art recognize the modified feature as still having the required characteristics and capabilities of the unmodified feature. In general, but subject to the preceding discussion, a numerical value herein that is modified by a word of approximation such as “about” may vary from the stated value by at least 1, 2, 3, 4, 5, 6, 7, 10, 12 or 15%.

Additionally, the section headings herein are provided for consistency with the suggestions under 37 CFR 1.77 or otherwise to provide organizational cues. These headings shall not limit or characterize the invention(s) set out in any claims that may issue from this disclosure. Specifically and by way of example, although the headings refer to a “Field of Invention,” such claims should not be limited by the language under this heading to describe the so-called technical field. Further, a description of technology in the “Background of the Invention” section is not to be construed as an admission that technology is prior art to any invention(s) in this disclosure. Neither is the “Summary” to be considered a characterization of the invention(s) set forth in issued claims. Furthermore, any reference in this disclosure to “invention” in the singular should not be used to argue that there is only a single point of novelty in this disclosure. Multiple inventions may be set forth according to the limitations of the multiple claims issuing from this disclosure, and such claims accordingly define the invention(s), and their equivalents, that are protected thereby. In all instances, the scope of such claims shall be considered on their own merits in light of this disclosure, but should not be constrained by the headings set forth herein.

All of the compositions and/or methods disclosed and claimed herein can be made and executed without undue experimentation in light of the present disclosure. While the compositions and methods of this invention have been described in terms of preferred embodiments, it will be apparent to those of skill in the art that variations may be applied to the compositions and/or methods and in the steps or in the sequence of steps of the method described herein without departing from the concept, spirit and scope of the invention. All such similar substitutes and modifications apparent to those skilled in the art are deemed to be within the spirit, scope and concept of the invention as defined by the appended claims.

To aid the Patent Office, and any readers of any patent issued on this application in interpreting the claims appended hereto, applicants wish to note that they do not intend any of the appended claims to invoke paragraph 6 of 35 U.S.C. § 112, U.S.C. § 112 paragraph (f), or equivalent, as it exists on the date of filing hereof unless the words “means for” or “step for” are explicitly used in the particular claim.

For each of the claims, each dependent claim can depend both from the independent claim and from each of the prior dependent claims for each and every claim so long as the prior claim provides a proper antecedent basis for a claim term or element.

### REFERENCES

1. Barcelo H, Faul J, Crimmins E, Thyagarajan B. A Practical Cryopreservation and Staining Protocol for Immunophenotyping in Population Studies. Curr Protoc Cytom. 2018; 84(1):e35. doi:10.1002/cpcy.35
2. Guillaumet-Adkins A, Rodriguez-Esteban G, Mereu E, et al. Single-cell transcriptome conservation in cryopreserved cells and tissues. Genome Biol. 2017; 18(1):45. doi:10.1186/s13059-017-1171-9
3. Williams J W, Winkels H, Durant C P, Zaitsev K, Ghosheh Y, Ley K. Single Cell RNA Sequencing in Atherosclerosis. Circ Res. 2020; In press. doi:10.1161/CIRCRESAHA.119.315940
4. Zheng C, Zheng L, Yoo J-K, et al. Landscape of Infiltrating T Cells in Liver Cancer Revealed by Single-Cell Sequencing. Cell. 2017; 169(7):1342-1356.e16. doi:10.1016/i.cell.2017.05.035
5. Zhang Y, Zheng L, Zhang L, Hu X, Ren X, Zhang Z. Deep single-cell RNA sequencing data of individual T cells from treatment-naive colorectal cancer patients. Sci data. 2019; 6(1):131. doi:10.1038/s41597-019-0131-5
6. Uniken Venema W T, Voskuil M D, Vila A V, et al. Single-Cell RNA Sequencing of Blood and Ileal T Cells From Patients With Crohn's Disease Reveals Tissue-Specific Characteristics and Drug Targets. Gastroenterology. 2019; 156(3):812-815.e22. doi:10.1053/j.gastro.2018.10.046
7. Reyes M, Vickers D, Billman K, et al. Multiplexed enrichment and genomic profiling of peripheral blood cells reveal subset-specific immune signatures. Sci Adv. 2019; 5(1):eaau9223. doi:10.1126/sciadv.aau9223
8. Kotliarov Y, Sparks R, Martins A J, et al. Broad immune activation underlies shared set point signatures for vaccine responsiveness in healthy individuals and disease activity in patients with lupus. Nat Med. 2020. doi:10.1038/s41591-020-0769-8
9. Fernandez D M, Rahman A H, Fernandez N F, et al. Single-cell immune landscape of human atherosclerotic plaques. Nat Med. 2019; 25(10):1576-1588.
10. Zheng G X Y, Terry J M, Belgrader P, et al. Massively parallel digital transcriptional profiling of single cells. Nat Commun. 2017; 8:14049. doi:10.1038/ncomms14049
11. Pizzolato G, Kaminski H, Tosolini M, et al. Single-cell RNA sequencing unveils the shared and the distinct cytotoxic hallmarks of human TCRVdelta1 and TCRVdelta2 gammadelta T lymphocytes. Proc Natl Acad Sci USA. 2019; 116(24):11906-11915. doi:10.1073/pnas.1818488116
12. Bossel Ben-Moshe N, Hen-Avivi S, Levitin N, et al. Predicting bacterial infection outcomes using single cell RNA-sequencing analysis of human immune cells. Nat Commun. 2019; 10(1):3266. doi:10.1038/s41467-019-11257-y
13. Stoeckius M, Hafemeister C, Stephenson W, et al. Simultaneous epitope and transcriptome measurement in single cells. Nat Methods. 2017; 14(9):865-868. doi:10.1038/nmeth.4380
14. Peterson V M, Zhang K X, Kumar N, et al. Multiplexed quantification of proteins and transcripts in single cells. Nat Biotechnol. 2017; 35(10):936-939. doi:10.1038/nbt.3973
15. Mair F, Erickson J R, Voillet V, et al. A Targeted Multi-omic Analysis Approach Measures Protein Expression and Low-Abundance Transcripts on the Single-Cell Level. Cell Rep. 2020; 31(1):107499. doi:10.1016/j.celrep.2020.03.063
16. Montaner J S G, Lima V D, Harrigan P R, et al. Expansion of HAART coverage is associated with sustained decreases in HIV/AIDS morbidity, mortality and HIV transmission: the “HIV Treatment as Prevention” experience in a Canadian setting. PLoS One. 2014; 9(2):e87872. doi:10.1371/journal.pone.0087872
17. Hanna D B, Lin J, Post W S, et al. Association of Macrophage Inflammation Biomarkers With Progression of Subclinical Carotid Artery Atherosclerosis in HIV-Infected Women and Men. J Infect Dis. 2017; 215(9):1352-1361. doi:10.1093/infdis/jix082
18. Mocroft A, Reiss P, Gasiorowski J, et al. Serious fatal and nonfatal non-AIDS-defining illnesses in Europe. J Acquir Immune Defic Syndr. 2010; 55(2):262-270. doi:10.1097/QAI.0b013e3181e9be6b
19. Neuhaus J, Angus B, Kowalska J D, et al. Risk of all-cause mortality associated with nonfatal AIDS and serious non-AIDS events among adults infected with HIV. AIDS. 2010; 24(5):697-706. doi:10.1097/QAD.0b013e3283365356
20. Wang Z, Usyk M, Sollecito C C, et al. Altered Gut Microbiota and Host Metabolite Profiles in HIV-infected Women. Clin Infect Dis. November 2019. doi:10.1093/cid/ciz1117
21. Hanna D B, Moon J-Y, Haberlen S A, et al. Carotid artery atherosclerosis is associated with mortality in HIV-positive women and men. AIDS. 2018; 32(16):2393-2403. doi:10.1097/QAD.0000000000001972
22. Hanna D B, Post W S, Deal J A, et al. HIV Infection Is Associated With Progression of Subclinical Carotid Atherosclerosis. Clin Infect Dis. 2015; 61(4):640-650. doi:10.1093/cid/civ325
23. Kuniholm M H, Hanna D B, Landay A L, Kaplan R C, Ley K. Soluble CD163 is associated with noninvasive measures of liver fibrosis in hepatitis C virus- and hepatitis C virus/human immunodeficiency virus-infected women. Hepatology. 2015; 61(2):734-735. doi:10.1002/hep.27303
24. Shaked I, Hanna D B, Gleissner C, et al. Macrophage inflammatory markers are associated with subclinical carotid artery disease in women with human immunodeficiency virus or hepatitis C virus infection. Arterioscler Thromb Vasc Biol. 2014; 34(5):1085-1092. doi:10.1161/ATVBAHA.113.303153
25. Mueller K A L, Hanna D B, Ehinger E, et al. Loss of CXCR4 on non-classical monocytes in participants of the Women's Interagency HIV Study (WIHS) with subclinical atherosclerosis. Cardiovasc Res. 2019; 115(6):1029-1040. doi:10.1093/cvr/cvy292
26. Westhorpe C L V, Maisa A, Spelman T, et al. Associations between surface markers on blood monocytes and carotid atherosclerosis in HIV-positive individuals. Immunol Cell Biol. 2014; 92(2):133-138. doi:10.1038/icb.2013.84
27. Saves M, Chene G, Ducimetiere P, et al. Risk factors for coronary heart disease in patients treated for human immunodeficiency virus infection compared with the general population. Clin Infect Dis. 2003; 37(2):292-298. doi:10.1086/375844
28. Kaplan R C, Kingsley L A, Sharrett A R, et al. Ten-year predicted coronary heart disease risk in HIV-infected men and women. Clin Infect Dis. 2007; 45(8):1074-1081. doi:10.1086/521935
29. Smit M, Brinkman K, Geerlings S, et al. Future challenges for clinical care of an ageing population infected with HIV: a modelling study. Lancet Infect Dis. 2015; 15(7):810-818. doi:10.1016/S1473-3099(15)00056-0
30. Twigg H L 3rd, Crystal R, Currier J, et al. Refining Current Scientific Priorities and Identifying New Scientific Gaps in HIV-Related Heart, Lung, Blood, and Sleep Research. AIDS Res Hum Retroviruses. 2017; 33(9):889-897. doi:10.1089/AID.2017.0026
31. Baker J V, Hullsiek K H, Singh A, et al. Immunologic predictors of coronary artery calcium progression in a contemporary HIV cohort. AIDS. 2014; 28(6):831-840. doi:10.1097/QAD.0000000000000145
32. Moir S, Fauci A S. B-cell responses to HIV infection. Immunol Rev. 2017; 275(1):33-48. doi:10.1111/imr.12502
33. Moir S, Fauci A S. B cells in HIV infection and disease. Nat Rev Immunol. 2009; 9(4):235-245. doi:10.1038/nri2524
34. Karnell J L, Kumar V, Wang J, Wang S, Voynova E, Ettinger R. Role of CD11c(+) T-bet(+) B cells in human health and disease. Cell Immunol. 2017; 321:40-45. doi:10.1016/j.cellimm.2017.05.008
35. Moir S, Fauci A S. B-cell exhaustion in HIV infection: the role of immune activation. Curr Opin HIV AIDS. 2014; 9(5):472-477. doi:10.1097/COH.0000000000000092
36. Hodis H N, Mack W J, Lobo R A, et al. Estrogen in the prevention of atherosclerosis. A randomized, double-blind, placebo-controlled trial. Ann Intern Med. 2001; 135(11):939-953. doi:10.7326/0003-4819-135-11-200112040-00005
37. Schechter M E, Andrade B B, He T, et al. Inflammatory monocytes expressing tissue factor drive SIV and HIV coagulopathy. Sci Transl Med. 2017; 9(405). doi:10.1126/scitranslmed.aam5441
38. Fan H C, Fu G K, Fodor SPA. Expression profiling. Combinatorial labeling of single cells for gene expression cytometry. Science. 2015; 347(6222). doi:10.1126/science.1258367
39. Ong S-M, Teng K, Newell E, et al. A Novel, Five-Marker Alternative to CD16−CD14 Gating to Identify the Three Human Monocyte Subsets. Front Immunol. 2019; 10:1761. doi:10.3389/fimmu.2019.01761
40. Spear T T, Nishimura M I, Simms P E. Comparative exploration of multidimensional flow cytometry software: a model approach evaluating T cell polyfunctional behavior. J Leukoc Biol. 2017; 102(2):551-561. doi:10.1189/jlb.6A0417-140R
41. Bryceson Y T, Fauriat C, Nunes J M, et al. Functional analysis of human NK cells by flow cytometry. Methods Mol Biol. 2010; 612:335-352. doi:10.1007/978-1-60761-362-6_23
42. Hamers A A J, Dinh H Q, Thomas G D, et al. Human Monocyte Heterogeneity as Revealed by High-Dimensional Mass Cytometry. Arterioscler Thromb Vasc Biol. 2019; 39(1):25-36. doi:10.1161/ATVBAHA.118.311022
43. Guilliams M, Mildner A, Yona S. Developmental and Functional Heterogeneity of Monocytes. Immunity. 2018; 49(4):595-613. doi:10.1016/j.immuni.2018.10.005
44. Spits H, Bernink J H, Lanier L. N K cells and type 1 innate lymphoid cells: partners in host defense. Nat Immunol. 2016; 17(7):758-764. doi:10.1038/ni.3482
45. Subelj L, Bajec M. Unfolding communities in large complex networks: combining defensive and offensive label propagation for core extraction. Phys Rev E Stat Nonlin Soft Matter Phys. 2011; 83(3 Pt 2):36103. doi:10.1103/PhysRevE.83.036103
46. Van Puijvelde G H M, Kuiper J. NKT cells in cardiovascular diseases. Eur J Pharmacol. 2017; 816(2017):47-57. doi:10.1016/j.ejphar.2017.03.052
47. Acosta-Rodriguez E V, Rivino L, Geginat J, et al. Surface phenotype and antigenic specificity of human interleukin 17-producing T helper memory cells. Nat Immunol. 2007; 8(6):639-646. doi:10.1038/ni1467
48. Emoto M, Zerrahn J, Miyamoto M, Perarnau B, Kaufmann S H. Phenotypic characterization of CD8(+)NKT cells. Eur J Immunol. 2000; 30(8):2300-2311. doi:10.1002/1521-4141(2000)30:8<2300::AID-IMMU2300>3.0.CO; 2-2
49. Seregin S S, Chen G Y, Laouar Y. Dissecting CD8+ NKT Cell Responses to Listeria Infection Reveals a Component of Innate Resistance. J Immunol. 2015; 195(3):1112-1120. doi:10.4049/jimmunol.1500084
50. Noels H, Weber C, Koenen R R. Chemokines as Therapeutic Targets in Cardiovascular Disease. Arterioscler Thromb Vasc Biol. 2019; 39(4):583-592. doi:10.1161/ATVBAHA.118.312037
51. Szentes V, Gazdag M, Szokodi I, Dezsi C A. The Role of CXCR3 and Associated Chemokines in the Development of Atherosclerosis and During Myocardial Infarction. Front Immunol. 2018; 9:1932. doi:10.3389/fimmu.2018.01932
52. SahBandar I N, Ndhlovu L C, Saiki K, et al. Relationship between Circulating Inflammatory Monocytes and Cardiovascular Disease Measures of Carotid Intimal Thickness. J Atheroscler Thromb. October 2019. doi:10.5551/jat.49791
53. Ruiz-Limon P, Ortega-Castro R, Barbarroja N, et al. Molecular Characterization of Monocyte Subsets Reveals Specific and Distinctive Molecular Signatures Associated With Cardiovascular Disease in Rheumatoid Arthritis. Front Immunol. 2019; 10:1111. doi:10.3389/fimmu.2019.01111
54. Narasimhan P B, Marcovecchio P, Hamers A A J, Hedrick C C. Nonclassical Monocytes in Health and Disease. Annu Rev Immunol. 2019; 37:439-456. doi:10.1146/annurev-immunol-042617-053119
55. Kaminski D A, Wei C, Qian Y, Rosenberg A F, Sanz I. Advances in human B cell phenotypic profiling. Front Immunol. 2012; 3:302. doi:10.3389/fimmu.2012.00302
56. Griffin D O, Rothstein T L. Human b1 cell frequency: isolation and analysis of human b1 cells. Front Immunol. 2012; 3:122. doi:10.3389/fimmu.2012.00122
57. Upadhye A, Srikakulapu P, Gonen A, et al. Diversification and CXCR4-Dependent Establishment of the Bone Marrow B-la Cell Pool Governs Atheroprotective IgM Production Linked to Human Coronary Atherosclerosis. Circ Res. 2019; 125(10):e55-e70. doi:10.1161/CIRCRESAHA.119.315786
58. Amu S, Tarkowski A, Dorner T, Bokarewa M, Brisslert M. The human immunomodulatory CD25+ B cell population belongs to the memory B cell pool. Scand J Immunol. 2007; 66(1):77-86. doi:10.1111/j.1365-3083.2007.01946.x
59. Moir S, Ho J, Malaspina A, et al. Evidence for HIV-associated B cell exhaustion in a dysfunctional memory B cell compartment in HIV-infected viremic individuals. J Exp Med. 2008; 205(8):1797-1805. doi:10.1084/jem.20072683
60. Bekele Y, Lakshmikanth T, Chen Y, et al. Mass cytometry identifies distinct CD4+ T cell clusters distinguishing HIV-1-infected patients according to antiretroviral therapy initiation. JCI insight. 2019; 4(3). doi:10.1172/jci.insight.125442
61. Kazer S W, Aicher T P, Muema D M, et al. Integrated single-cell analysis of multicellular immune dynamics during hyperacute HIV-1 infection. Nat Med. March 2020. doi:10.1038/s41591-020-0799-2
62. Albany C J, Trevelin S C, Giganti G, Lombardi G, Scotta C. Getting to the Heart of the Matter: The Role of Regulatory T-Cells (Tregs) in Cardiovascular Disease (CVD) and Atherosclerosis. Front Immunol. 2019; 10:2795. doi:10.3389/fimmu.2019.02795
63. Bjorkbacka H. Can Circulating Regulatory T Cells Predict Cardiovascular Disease? EBioMedicine. 2016; 11:15-16. doi:10.1016/j.ebiom.2016.08.044
64. Wigren M, Bjorkbacka H, Andersson L, et al. Low levels of circulating CD4+FoxP3+ T cells are associated with an increased risk for development of myocardial infarction but not for stroke. Arterioscler Thromb Vasc Biol. 2012; 32(8):2000-2004. doi:10.1161/ATVBAHA.112.251579
65. Jansen H, Koenig W, Jaensch A, et al. Prognostic Utility of Galectin-3 for Recurrent Cardiovascular Events During Long-term Follow-up in Patients with Stable Coronary Heart Disease: Results of the KAROLA Study. Clin Chem. 2016; 62(10):1372-1379. doi:10.1373/clinchem.2016.257550
66. Ghorbani A, Bhambhani V, Christenson R H, et al. Longitudinal Change in Galectin-3 and Incident Cardiovascular Outcomes. J Am Coll Cardiol. 2018; 72(25):3246-3254. doi:10.1016/j.jacc.2018.09.076
67. Winkels H, Ehinger E, Vassallo M, et al. Atlas of the immune cell repertoire in mouse atherosclerosis defined by single-cell RNA-sequencing and mass cytometry. Circ Res. 2018; 122(12):1675-1688. doi:10.1161/CIRCRESAHA.117.312513
68. Cole J E, Park I, Ahern D J, et al. Immune cell census in murine atherosclerosis: cytometry by time of flight illuminates vascular myeloid cell diversity. Cardiovasc Res. 2018; 114(10):1360-1371. doi:10.1093/cvr/cvy109
69. Spitzer M H, Nolan G P. Mass Cytometry: Single Cells, Many Features. Cell. 2016; 165(4):780-791. doi:10.1016/j.cell.2016.04.019
70. Robinson J P, Roederer M. HISTORY O F SCIENCE. Flow cytometry strikes gold. Science. 2015; 350(6262):739-740. doi:10.1126/science.aad6770
71. Roy B, Haupt L M, Griffiths L R. Alternative Splicing (A S) of Genes As An Approach for Generating Protein Complexity. Curr Genomics. 2013; 14(3):182-194. doi:10.2174/1389202911314030004
72. Moremen K W, Tiemeyer M, Naim A V. Vertebrate protein glycosylation: diversity, synthesis and function. Nat Rev Mol Cell Biol. 2012; 13(7):448-462. doi:10.1038/nrm3383
73. Battle A, Khan Z, Wang S H, et al. Genomic variation. Impact of regulatory variation from RNA to protein. Science. 2015; 347(6222):664-667. doi:10.1126/science.1260793
74. Hinkson I V, Elias J E. The dynamic state of protein turnover: It's about time. Trends Cell Biol. 2011; 21(5):293-303. doi:10.1016/j.tcb.2011.02.002
75. Liu Y, Beyer A, Aebersold R. On the Dependency of Cellular Protein Levels on mRNA Abundance. Cell. 2016; 165(3):535-550. doi:10.1016/j.cell.2016.03.014
76. Lundberg E, Fagerberg L, Klevebring D, et al. Defining the transcriptome and proteome in three functionally different human cell lines. Mol Syst Biol. 2010; 6:450. doi:10.1038/msb.2010.106
77. Cochain C, Vafadamejad E, Arampatzi P, et al. Single-Cell RNA-Seq Reveals the Transcriptional Landscape and Heterogeneity of Aortic Macrophages in Murine Atherosclerosis. Circ Res. 2018; 122(12):1661-1674. doi:10.1161/CIRCRESAHA.117.312509
78. Gao L, Wang Y, Li Y, et al. Genome-wide expression profiling analysis to identify key genes in the anti-HIV mechanism of CD4(+) and CD8(+) T cells. J Med Virol. 2018; 90(7):1199-1209. doi:10.1002/jmv.25071
79. Kolbus D, Ljungcrantz I, Andersson L, et al. Association between CD8+ T-cell subsets and cardiovascular disease. J Intern Med. 2013; 274(1):41-51. doi:10.1111/joim.12038
80. Cochain C, Zernecke A. Protective and pathogenic roles of CD8(+) T cells in atherosclerosis. Basic Res Cardiol. 2016; 111(6):71. doi:10.1007/s00395-016-0589-7
81. Fernandez C S, Kelleher A D, Finlayson R, Godfrey D I, Kent S J. NKT cell depletion in humans during early HIV infection. Immunol Cell Biol. 2014; 92(7):578-590. doi:10.1038/icb.2014.25
82. Moll M, Kuylenstierna C, Gonzalez V D, et al. Severe functional impairment and elevated PD-1 expression in CD1d-restricted NKT cells retained during chronic HIV-1 infection. Eur J Immunol. 2009; 39(3):902-911. doi:10.1002/eji.200838780
83. Vasan S, Poles M A, Horowitz A, Siladji E E, Markowitz M, Tsuji M. Function of NKT cells, potential anti-HIV effector cells, are improved by beginning HAART during acute HIV-1 infection. Int Immunol. 2007; 19(8):943-951. doi:10.1093/intimm/dxm055
84. Flynn M C, Pernes G, Lee M K S, Nagareddy P R, Murphy A J. Monocytes, Macrophages, and Metabolic Disease in Atherosclerosis. Front Pharmacol. 2019; 10:666. doi:10.3389/fphar.2019.00666
85. Combadiere C, Potteaux S, Rodero M, et al. Combined inhibition of CCL2, CX3CR1, and CCR5 abrogates Ly6C(hi) and Ly6C(lo) monocytosis and almost abolishes atherosclerosis in hypercholesterolemic mice. Circulation. 2008; 117(13):1649-1657. doi:10.1161/CIRCULATIONAHA.107.745091
86. Quintar A, McArdle S, Wolf D, et al. Endothelial Protective Monocyte Patrolling in Large Arteries Intensified by Western Diet and Atherosclerosis. Circ Res. 2017; 120(11):1789-1799. doi:10.1161/CIRCRESAHA.117.310739
87. Moir S, Buckner C M, Ho J, et al. B cells in early and chronic HIV infection: evidence for preservation of immune function associated with early initiation of antiretroviral therapy. Blood. 2010; 116(25):5571-5579. doi:10.1182/blood-2010-05-285528
88. Nour-Eldine W, Joffre J, Zibara K, et al. Genetic Depletion or Hyperresponsiveness of Natural Killer Cells Do Not Affect Atherosclerosis Development. Circ Res. 2018; 122(1):47-57. doi:10.1161/CIRCRESAHA.117.311743
89. Bonaccorsi I, Spinelli D, Cantoni C, et al. Symptomatic Carotid Atherosclerotic Plaques Are Associated With Increased Infiltration of Natural Killer (NK) Cells and Higher Serum Levels of NK Activating Receptor Ligands. Front Immunol. 2019; 10:1503. doi:10.3389/fimmu.2019.01503
90. Barkan S E, Melnick S L, Preston-Martin S, et al. The Women's Interagency HIV Study.

WIHS Collaborative Study Group. Epidemiology. 1998; 9(2):117-125.
91. Bacon M C, von Wyl V, Alden C, et al. The Women's Interagency HIV Study: an observational cohort brings clinical sciences to the bench. Clin Diagn Lab Immunol. 2005; 12(9):1013-1019. doi:10.1128/CDLI.12.9.1013-1019.2005
92. Kaplan R C, Kingsley L A, Gange S J, et al. Low CD4+ T-cell count as a major atherosclerosis risk factor in HIV-infected women and men. AIDS. 2008; 22(13):1615-1624. doi:10.1097/QAD.0b013e328300581d
93. Olson N C, Doyle M F, Sitlani C M, et al. Associations of Innate and Adaptive Immune Cell Subsets With Incident Type 2 Diabetes Risk: The MESA Study. J Clin Endocrinol Metab. 2020; 105(3). doi:10.1210/clinem/dgaa036
94. Bittner D O, Mayrhofer T, Puchner S B, et al. Coronary Computed Tomography Angiography-Specific Definitions of High-Risk Plaque Features Improve Detection of Acute Coronary Syndrome. Circ Cardiovasc Imaging. 2018; 11(8):e007657. doi:10.1161/CIRCIMAGING.118.007657
95. Gaemperli O, Kaufmann P A. PET and PET/C T in cardiovascular disease. Ann N Y Acad Sci. 2011; 1228:109-136. doi:10.1111/j.1749-6632.2011.06030.x
96. Berry J D, Dyer A, Cai X, et al. Lifetime risks of cardiovascular disease. N Engl J Med. 2012; 366(4):321-329. doi:10.1056/NEJMoa1012848
97. Fowkes F G R, Aboyans V, Fowkes F J I, McDermott M M, Sampson U K A, Criqui M H. Peripheral artery disease: epidemiology and global perspectives. Nat Rev Cardiol. 2017; 14(3):156-170. doi:10.1038/nrcardio.2016.179
98. Rosenberg A B, Roco C M, Muscat R A, et al. Single-cell profiling of the developing mouse brain and spinal cord with split-pool barcoding. Science. 2018; 360(6385):176-182. doi:10.1126/science.aam8999
99. Rohlenova K, Goveia J, Garcia-Caballero M, et al. Single-Cell RNA Sequencing Maps Endothelial Metabolic Plasticity in Pathological Angiogenesis. Cell Metab. 2020; 31(4):862-877.e14. doi:10.1016/j.cmet.2020.03.009
100. Trapnell C, Pachter L, Salzberg S L. TopHat: discovering splice junctions with RNA-Seq. Bioinformatics. 2009; 25(9):1105-1111. doi:10.1093/bioinformatics/btp120
101. Wang L, Wang S, Li W. RSeQC: quality control of RNA-seq experiments. Bioinformatics. 2012; 28(16):2184-2185. doi:10.1093/bioinformatics/bts356
102. Robinson M D, McCarthy D J, Smyth G K. edge a Bioconductor package for differential expression analysis of digital gene expression data. Bioinformatics. 2010; 26(1):139-140. doi:10.1093/bioinformatics/btp616
103. Milush J M, Long B R, Snyder-Cappione J E, et al. Functionally distinct subsets of human NK cells and monocyte/DC-like cells identified by coexpression of CD56, CD7, and CD4. Blood. 2009; 114(23):4823-4831. doi:10.1182/blood-2009-04-216374

What is claimed is:

1. A method to determine a risk of a cardiovascular disease (CVD) or a subclinical CVD (sCVD) in a subject, the method comprising:
(a) obtaining a sample from the subject;
(b) isolating immune cells from the sample;
(c) detecting a first (INT1), second (INT2), third (INT3) and fourth (INT4) subset of intermediate monocyte cell populations in the immune cells, wherein a percentage of the INT1, INT2, INT3, and INT4 subsets as compared to a total intermediate monocyte cell population present in the isolated immune cells is determined;
(d) comparing a proportion of the INT1, INT2, INT3, and INT4 subsets subset of intermediate monocyte cell populations determined in (c) with a statistical sample representative of a proportion of equivalent INT1, INT2, INT3, and INT4 subsets in a total intermediate monocyte cell populations from a subject that does not have a cardiovascular disease; and
(e) determining that the subject has an increased risk for cardiovascular disease where the subject has a decrease in the INT2 and INT3 subsets, has an increase in the INT4 subset, or both, as compared to the statistical sample.

2. The method of claim 1, wherein if the subject is at increased risk based on the comparing step, then further treating the subject for cardiovascular disease.

3. The method of claim 1, wherein the intermediate monocyte cell population is detected in the isolated immune cells by gating for CD3−CD19−CD14+CD16+CD56− immune cells.

4. The method of claim 1, wherein the INT2 subset is detected by gating the isolated immune cells for IL6RA+, CD69+, CD86+, CD9+, CD141+, CD142+, CD163+, CCR2+, CCR4+, CCR5+, CD36+, CD38+, CD45RO+, CD206+.

5. The method of claim 1, wherein the INT3 subset is detected by gating the isolated immune cells for IL6RA, CD141+, CD142+, CD163+, CCR2+, CD36+, CD38+, CD69+, CD86+CD9+, CD154, CCR4+, CD69+, and optionally LAG3+, CD206+, CD137+, CCR4+, CCR5+, CD206, CD45RO+, CD4.

6. The method of claim 1, wherein the INT4 subset is detected by gating the isolated immune cells for IL6RA_{low}, CD137^{lower than INT 2 and 3}, CD154−, CD163^{low}, CCR2^{low}, CCR4^{low}, CCR5^{low}, CD206^{low}, LAG3−, CD36^{lower than INT2 and 3}, CD38^{low}, and optionally CD45RA, CD9+.

7. The method of claim 1, wherein the subject is determined to have a decrease in the INT2 subset as compared to the statistical sample, where the proportion of the INT2 subset is 40% of a total CD3−CD19−CD14+CD16+CD56− INT population in the statistical sample and less than 20% of a total CD3−CD19−CD14+CD16+CD56− INT population in the subject.

8. The method of claim 1, wherein the subject is determined to have a decrease in the INT2 subset as compared to the statistical sample where there is one half of an amount of INT2 cells present in the subject as compared to the statistical sample.

9. The method of claim 1, wherein the subject is determined to have a decrease in the INT3 subset as compared to the statistical sample, where the proportion of the INT3 subset is 30% of a total CD3−CD19−CD14+CD16+CD56− INT population in the statistical sample and 10% of the total CD3−CD19−CD14+CD16+CD56− INT population in the subject.

10. The method of claim 1, wherein the subject is determined to have a decrease in the INT3 subset as compared to the statistical sample where there is one-third of an amount of INT3 cells present in the subject as compared to the statistical sample.

11. The method of claim 1, wherein the subject is determined to have an increase in the INT4 subset as compared to the statistical sample, where the proportion of the INT4 subset is 12% of a total CD3−CD19−CD14+CD16+CD56− INT population in the statistical sample and 40% of a total CD3−CD19−CD14+CD16+CD56− INT population in the subject.

12. The method of claim 1, wherein the subject is determined to have an increase in the INT4 subset as compared to the statistical sample where there is roughly four times an amount INT4 cells present in the subject as compared to the statistical sample.

13. The method of claim 1, wherein the sample is a blood, serum, or plasma sample, and biomarkers are detected as a protein or a nucleic acid.

14. A method for treating a cardiovascular disease (CVD) or a subclinical CVD (sCVD) in a subject, the method comprising:
(a) obtaining a sample from the subject;
(b) isolating immune cells from the sample;
(c) detecting a first (INT1), second (INT2), third (INT3) and fourth (INT4) subset of intermediate monocyte cell populations in the immune cells, wherein a percentage of the INT1, INT2, INT3, and INT4 subsets as compared to a total intermediate monocyte cell population present in the isolated immune cells is determined;
(d) comparing a proportion of the INT1, INT2, INT3, and INT4 subsets subset of intermediate monocyte cell populations determined in (c) with a statistical sample representative of the proportion of equivalent INT1, INT2, INT3, and INT4 subsets in the total intermediate monocyte cell populations from a subject that does not have a cardiovascular disease;
(e) determining that the subject has a CVD or subclinical CVD, wherein if the subject has a decrease in the INT2 and INT3 subsets, has an increase in the INT4 subset, or both, as compared to the statistical sample, it is determined that the subject has a CVD or a subclinical CVD; and
(g) where the subject has a CVD or a subclinical CVD based on the comparing step, then further providing the subject with a treatment that prevents or treats the cardiovascular disease.

15. The method of claim 14, wherein the intermediate monocyte cell population is detected in the isolated immune cells by gating for CD3−CD19−CD14+CD16+CD56− immune cells.

16. The method of claim 14, wherein the INT2 subset is detected by gating the isolated immune cells for IL6RA+, CD69+, CD86+, CD9+, CD141+, CD142+, CD163+, CCR2+, CCR4+, CCR5+, CD36+, CD38+, CD45RO+, CD206+.

17. The method of claim 14, wherein the INT3 subset is detected by gating the isolated immune cells for IL6RA, CD141+, CD142+, CD163+, CCR2+, CD36+, CD38+, CD69+, CD86+CD9+, CD154, CCR4+, CD69+, and optionally LAG3+, CD206+, CD137+, CCR4+, CCR5+, CD206, CD45RO+, CD4.

18. The method of claim 14, wherein the INT4 subset is detected by gating the isolated immune cells for IL6RA_{low}, CD137^{lower than INT 2 and 3}, CD154−, CD163^{low}, CCR2^{low}, CCR4^{low}, CCR5^{low}, CD206^{low}, LAG3−, CD36^{lower than INT2 and 3}, CD38^{low}, and optionally CD45RA, CD9+.

19. The method of claim 14, wherein the subject is determined to have a decrease in the INT2 subset as compared to the statistical sample, where the proportion of the INT2 subset is 40% of a total CD3−CD19−CD14+CD16+CD56− INT population in the statistical sample and less than 20% of a total CD3−CD19−CD14+CD16+CD56− INT population in the subject.

20. The method of claim 14, wherein the subject is determined to have a decrease in the INT2 subset as compared to the statistical sample where there is one half of an amount of INT2 cells present in the subject as compared to the statistical sample.

21. The method of claim 14, wherein the subject is determined to have a decrease in the INT3 subset as compared to the statistical sample, where the proportion of the INT3 subset is 30% of a total CD3−CD19−CD14+CD16+CD56− INT population in the statistical sample and 10% of the total CD3−CD19−CD14+CD16+CD56− INT population in the subject.

22. The method of claim 14, wherein the subject is determined to have a decrease in the INT3 subset as compared to the statistical sample where there is one-third of an amount of INT3 cells present in the subject as compared to the statistical sample.

23. The method of claim 14, wherein the subject is determined to have an increase in the INT4 subset as compared to the statistical sample, where the proportion of the INT4 subset is 12% of a total CD3−CD19−CD14+CD16+CD56− INT population in the statistical sample and 40% of a total CD3−CD19−CD14+CD16+CD56− INT population in the subject.

24. The method of claim 14, wherein the subject is determined to have an increase in the INT4 subset as compared to the statistical sample where there is roughly four times an amount INT4 cells present in the subject as compared to the statistical sample.

25. The method of claim 14, wherein the sample is a blood, serum, or plasma sample, and biomarkers are detected as a protein or a nucleic acid.

26. A method of diagnosing a cardiovascular disease (CVD) or a subclinical CVD (sCVD) in a subject, the method comprising:
(a) obtaining a sample from the subject;
(b) isolating immune cells from the sample;
(c) detecting a first (INT1), second (INT2), third (INT3) and fourth (INT4) subset of intermediate monocyte cell populations in the immune cells, wherein a percentage of the INT1, INT2, INT3, and INT4 subsets as compared to a total intermediate monocyte cell population present in the isolated immune cells is determined;
(d) comparing a proportion of the INT1, INT2, INT3, and INT4 subsets subset of intermediate monocyte cell populations determined in (c) with a statistical sample representative of the proportion of equivalent INT1, INT2, INT3, and INT4 subsets in the total intermediate monocyte cell populations from a subject that does not have a cardiovascular disease; and
(e) determining that the subject has a cardiovascular disease or subclinical CVD, wherein if the subject has a decrease in the INT2 and INT3 subsets, has an increase in the INT4 subset, or both, as compared to the statistical sample, it is determined that the subject will develop or has a cardiovascular disease.

27. The method of claim 26, wherein the intermediate monocyte cell population is detected in the isolated immune cells by gating for CD3−CD19−CD14+CD16+CD56− immune cells.

28. The method of claim 26, wherein the INT2 subset is detected by gating the isolated immune cells for IL6RA+, CD69+, CD86+, CD9+, CD141+, CD142+, CD163+, CCR2+, CCR4+, CCR5+, CD36+, CD38+, CD45RO+, CD206+.

29. The method of claim 26, wherein the INT3 subset is detected by gating the isolated immune cells for IL6RA, CD141+, CD142+, CD163+, CCR2+, CD36+, CD38+, CD69+, CD86+CD9+, CD154, CCR4+, CD69+, and optionally LAG3+, CD206+, CD137+, CCR4+, CCR5+, CD206, CD45RO+, CD4.

30. The method of claim 1, wherein the INT4 subset is detected by gating the isolated immune cells for IL6RA_{low}, CD137^{lower than INT 2 and 3}, CD154−, CD163^{low}, CCR2^{low}, CCR4^{low}, CCR5^{low}, CD206^{low}, LAG3−, CD36^{lower than INT2 and 3}, CD38^{low}, and optionally CD45RA, CD9+.

31. The method of claim 26, wherein the subject is determined to have a decrease in the INT2 subset as compared to the statistical sample, where the proportion of the INT2 subset is 40% of a total CD3−CD19− CD14+CD16+CD56− INT population in the statistical sample and less than 20% of a total CD3−CD19−CD14+CD16+CD56− INT population in the subject.

32. The method of claim 26, wherein the subject is determined to have a decrease in the INT2 subset as compared to the statistical sample where there is one half of an amount of INT2 cells present in the subject as compared to the statistical sample.

33. The method of claim 26, wherein the subject is determined to have a decrease in the INT3 subset as compared to the statistical sample, where the proportion of the INT3 subset is 30% of a total CD3−CD19−CD14+CD16+CD56− INT population in the statistical sample and 10% of the total CD3−CD19−CD14+CD16+CD56− INT population in the subject.

34. The method of claim 26, wherein the subject is determined to have a decrease in the INT3 subset as compared to the statistical sample where there is one-third of an amount of INT3 cells present in the subject as compared to the statistical sample.

35. The method of claim 26, wherein the subject is determined to have an increase in the INT4 subset as compared to the statistical sample, where the proportion of the INT4 subset is 12% of a total CD3−CD19−CD14+CD16+CD56− INT population in the statistical sample and 40% of a total CD3−CD19−CD14+CD16+CD56− INT population in the subject.

36. The method of claim 26, wherein the subject is determined to have an increase in the INT4 subset as compared to the statistical sample where there is roughly four times an amount INT4 cells present in the subject as compared to the statistical sample.

37. The method of claim 26, wherein the sample is a blood, serum, or plasma sample, and biomarkers are detected as a protein or a nucleic acid.

38. A method to determine whether a subject needs treatment for a cardiovascular disease (CVD) or a subclinical CVD (sCVD), the method comprising:
(a) obtaining a sample from the subject;
(b) isolating the immune cells from the sample;
(c) measuring the presence of one or more immune cell subpopulations from the isolated immune cells, and an expression level of one or more biomarkers in the one or more immune cell subpopulations;
(d) comparing the expression level of the one or more biomarkers in (c) with a statistical sample representative of the biomarkers present in an equivalent immune cell subpopulation of a subject not having a cardiovascular disease; and
(e) determining that the subject is to be treated for the cardiovascular disease based on the comparing step of (d), wherein if the expression level of the one or more biomarkers present in the subject sample is higher than a baseline level of the statistical sample, the subject is determined to need treatment for the CVD or subclinical CVD.

39. The method of claim 38, further comprising treating the subject in need of treatment for cardiovascular disease or subclinical CVD.

40. The method of claim 38, wherein the one or more immune cell subpopulations is selected from CD4 T cells or subpopulations thereof, CD8 T cells or subpopulations thereof, intermediate monocytes or subpopulations thereof, B cells or subpopulations thereof, and Natural Killer cells or subpopulations thereof.

41. The method of claim 38, wherein the sample is a blood, serum, or plasma sample, and the biomarkers are detected as a protein or a nucleic acid.

42. The method of claim 38, wherein the immune cell subpopulation consists essentially of T cells and the one or more biomarkers are selected from 1, 2, 3, 4, 5, 6, 7, 8, 9, 10, 15, 20, 25, 30, 40, or 50 biomarkers selected from: SELPLG; IFITM3; MYC; FYB; JUNB; IL32; SELL; CCR7; GIMAP5; TCF7; FAS; BCL6; TNFSF10; STAT3; CD27; BIRC3; IKZF1; PTPRC; CYTIP; IFITM2; CD2; LCK; IL7R; FOXO1; TRAC; CCA; CD69; ICOS; SLC2A3; LEF1; RGS1; IL4R; CD52; HOPX; GZMA; KLRG1; KLRD1; TIGIT; STAT1; KLRC1; LGALS1; KLRC4; CD63; SAMD3; S100A10; KLRK1; IL23R; CCL5; APOBEC3G; CD160; TARP; CD3D; CD8A; KLRD1; ITGA4; CX3CR1; VNN2; GZMA; CH3L2; or GIMAP5, and optionally the biomarkers are selected in the order listed.

43. The method of claim 38, wherein the immune cell subpopulation consists essentially of classical monocytes and the one or more biomarkers are selected from 1, 2, 3, 4, 5, 6, 7, 8, 9, 10, 15, 20, 25, 30, or 40 biomarkers selected from: CCL4; CCL3; SLC2A3; SOD2; SELPLG; CD14; LAP3; FYB; LYN; CD80; TNFSF10; TNFSF8; CSF3; CSF3R; CD36; NAIP; SELL; CLEC4E; IL8R; PTPRC; VCAN; ADGRE1; MNDA; DUSP1; FYB; DUSP2; LYZ; JUNB; SCREP1; DOCK8; CD300A; LAP3; TNFSF13; SDCP; MDX1; FYN; STAT6; IL1B; NAMPT; STAT3; IL6; IER3; TLR2; CD83; FYB; S100A9; TNFSF13B; CLEC4E; or ICAM1, and optionally the biomarkers are selected in the order listed.

44. The method of claim 38, wherein the immune cell subpopulation consists essentially of intermediate monocytes and the one or more biomarkers are selected from 1, 2, 3, 4, 5, 6, 7, 8, 9, or 10 biomarkers selected from: PRDM1; TNF; CCL4; LYZ; DUSP1; IL1B; DUSP2; CCL3; IER3; or ICAM1, and optionally the biomarkers are selected in the order listed.

45. The method of claim 38, wherein the immune cell subpopulation consists essentially of B cells and the one or more biomarkers are selected from 1, 2, 3, 4, 5, 6, 7, 8, or 9 biomarkers selected from: CD74; IGHD; CD79A; LYN; CD83; R3HDM4; CD79B; CXCR5; or SLC2A3, and optionally the biomarkers are selected in the order listed.

46. The method of claim 38, wherein the cardiovascular disease is selected from at least one of: coronary heart disease (CHD), heart failure (HF), coronary heart disease (CHD), cerebrovascular disease, peripheral arterial disease, atherosclerosis, or heart failure.

47. A method to prevent cardiovascular disease (CVD) in a subject with unknown CVD status, the method comprising:
(a) obtaining a sample from the subject;
(b) isolating immune cells from the sample;
(c) measuring a presence of one or more immune cell subpopulations from the isolated immune cells, and determining an expression level of one or more biomarkers in the one or more immune cell subpopulations;
(d) comparing the expression level of the one or more biomarkers in (c) with a statistical sample representative of the biomarkers in an equivalent immune cell subpopulation of a subject not having a cardiovascular disease;
(e) determining the CVD status of the subject based on the comparing step of (d), wherein if the expression level of the one or more biomarkers present in the subject sample is higher than a baseline level of the statistical sample, the subject is determined to need treatment to prevent CVD; and
(f) administering a treatment to prevent CVD in the subject.

48. The method of claim 47, wherein the treatment to prevent CVD comprises administering a statin.

49. The method of claim 47, wherein the one or more immune cell subpopulation is selected from CD4 T cells or subpopulations thereof, CD8 T cells or subpopulations thereof, intermediate monocytes or subpopulations thereof, B cells or subpopulations thereof, and Natural Killer cells or subpopulations thereof.

50. The method of claim 47, wherein the sample is a blood, serum, or plasma sample, and the biomarkers are detected as a protein or a nucleic acid.

51. The method of claim 47, wherein the immune cell subpopulation consists essentially of T cells and the one or more biomarkers are selected from 1, 2, 3, 4, 5, 6, 7, 8, 9, 10, 15, 20, 25, 30, 40, or 50 biomarkers selected from: SELPLG; IFITM3; MYC; FYB; JUNB; IL32; SELL; CCR7; GIMAP5; TCF7; FAS; BCL6; TNFSF10; STAT3; CD27; BIRC3; IKZF1; PTPRC; CYTIP; IFITM2; CD2; LCK; IL7R; FOXO1; TRAC; CCA; CD69; ICOS; SLC2A3; LEF1; RGS1; IL4R; CD52; HOPX; GZMA; KLRG1; KLRD1; TIGIT; STAT1; KLRC1; LGALS1; KLRC4; CD63; SAMD3; S100A10; KLRK1; IL23R; CCL5; APOBEC3G; CD160; TARP; CD3D; CD8A; KLRD1; ITGA4; CX3CR1; VNN2; GZMA; CH3L2; or GIMAP5, and optionally the biomarkers are selected in the order listed.

52. The method of claim 47, wherein the immune cell subpopulation consists essentially of classical monocytes and the one or more biomarkers are selected from 1, 2, 3, 4, 5, 6, 7, 8, 9, 10, 15, 20, 25, 30, or 40 biomarkers selected from: CCL4; CCL3; SLC2A3; SOD2; SELPLG; CD14; LAP3; FYB; LYN; CD80; TNFSF10; TNFSF8; CSF3; CSF3R; CD36; NAIP; SELL; CLEC4E; IL8R; PTPRC; VCAN; ADGRE1; MNDA; DUSP1; FYB; DUSP2; LYZ; JUNB; SCREP1; DOCK8; CD300A; LAP3; TNFSF13; SDCP; MDX1; FYN; STAT6; IL1B; NAMPT; STAT3; IL6; IER3; TLR2; CD83; FYB; S100A9; TNFSF13B; CLEC4E; or ICAM1, and optionally the biomarkers are selected in the order listed.

53. The method of claim 47, wherein the immune cell subpopulation consists essentially of intermediate monocytes and the one or more biomarkers are selected from 1, 2, 3, 4, 5, 6, 7, 8, 9, or 10 biomarkers selected from: PRDM1; TNF; CCL4; LYZ; DUSP1; IL1B; DUSP2; CCL3; IER3; or ICAM1, and optionally the biomarkers are selected in the order listed.

54. The method of claim 47, wherein the immune cell subpopulation consists essentially of B cells and the one or more biomarkers are selected from 1, 2, 3, 4, 5, 6, 7, 8, or 9 biomarkers selected from: CD74; IGHD; CD79A; LYN; CD83; R3HDM4; CD79B; CXCR5; or SLC2A3, and optionally the biomarkers are selected in the order listed.

55. The method of claim 47, wherein the cardiovascular disease is selected from at least one of: coronary heart disease (CHD), heart failure (HF), coronary heart disease (CHD), cerebrovascular disease, peripheral arterial disease, atherosclerosis, or heart failure.

56. A method of treating a subject that has or will develop a cardiovascular disease, the method comprising:
(a) obtaining a sample from the subject;
(b) isolating immune cells from the sample;
(c) measuring a presence of one or more immune cell subpopulations from the isolated immune cells, and an expression level of one or more biomarkers in the one or more immune cell subpopulations;
(d) comparing the expression level of the one or more biomarkers in (c) with a statistical sample representative of the biomarkers in an equivalent immune cell subpopulation of a subject not having a cardiovascular disease; and
(e) ruling-out the subject as having cardiovascular disease from a diagnostic test for cardiovascular disease, a treatment of cardiovascular disease, or a combination thereof, based on the comparing step of (d), wherein if the expression level of the one or more biomarkers present in the subject sample is lower than a baseline level of the statistical sample, the subject is determined not to have or be likely to develop CVD; or
(f) administering a preventive treatment for the cardiovascular disease, a treatment for cardiovascular disease, or a combination thereof to the subject who is at risk of developing the cardiovascular disease based on the comparing step of (d), wherein if the expression level of the one or more biomarkers present in the subject sample is higher than a baseline level of the statistical sample, the subject is to have or be likely to develop CVD.

57. The method of claim 56, wherein the one or more immune cell subpopulations is selected from CD4 T cells or subpopulations thereof, CD8 T cells or subpopulations thereof, intermediate monocytes or subpopulations thereof, B cells or subpopulations thereof, and Natural Killer cells or subpopulations thereof.

58. The method of claim 56, wherein the sample is a blood, serum, or plasma sample, and the biomarkers are detected as a protein or a nucleic acid.

59. The method of claim 56, wherein the immune cell subpopulation consists essentially of T cells and the one or more biomarkers are selected from 1, 2, 3, 4, 5, 6, 7, 8, 9, 10, 15, 20, 25, 30, 40, or 50 biomarkers selected from: SELPLG; IFITM3; MYC; FYB; JUNB; IL32; SELL; CCR7; GIMAP5; TCF7; FAS; BCL6; TNFSF10; STAT3; CD27; BIRC3; IKZF1; PTPRC; CYTIP; IFITM2; CD2; LCK; IL7R; FOXO1; TRAC; CCA; CD69; ICOS; SLC2A3; LEF1; RGS1; IL4R; CD52; HOPX; GZMA; KLRG1; KLRD1; TIGIT; STAT1; KLRC1; LGALS1; KLRC4; CD63; SAMD3; S100A10; KLRK1; IL23R; CCL5; APOBEC3G; CD160; TARP; CD3D; CD8A; KLRD1; ITGA4; CX3CR1; VNN2; GZMA; CH3L2; or GIMAP5, and optionally the biomarkers are selected in the order listed.

60. The method of claim 56, wherein the immune cell subpopulation consists essentially of classical monocytes and the one or more biomarkers are selected from 1, 2, 3, 4, 5, 6, 7, 8, 9, 10, 15, 20, 25, 30, or 40 biomarkers selected from: CCL4; CCL3; SLC2A3; SOD2; SELPLG; CD14; LAP3; FYB; LYN; CD80; TNFSF10; TNFSF8; CSF3; CSF3R; CD36; NAIP; SELL; CLEC4E; IL8R; PTPRC; VCAN; ADGRE1; MNDA; DUSP1; FYB; DUSP2; LYZ; JUNB; SCREP1; DOCK8; CD300A; LAP3; TNFSF13; SDCP; MDX1; FYN; STAT6; IL1B; NAMPT; STAT3; IL6; IER3; TLR2; CD83; FYB; S100A9; TNFSF13B; CLEC4E; or ICAM1, and optionally the biomarkers are selected in the order listed.

61. The method of claim 56, wherein the immune cell subpopulation consists essentially of intermediate monocytes and the one or more biomarkers are selected from 1, 2, 3, 4, 5, 6, 7, 8, 9, or 10 biomarkers selected from: PRDM1; TNF; CCL4; LYZ; DUSP1; IL1B; DUSP2; CCL3; IER3; or ICAM1, and optionally the biomarkers are selected in the order listed.

62. The method of claim 56, wherein the immune cell subpopulation consists essentially of B cells and the one or more biomarkers are selected from 1, 2, 3, 4, 5, 6, 7, 8, or 9 biomarkers selected from: CD74; IGHD; CD79A; LYN; CD83; R3HDM4; CD79B; CXCR5; or SLC2A3, and optionally the biomarkers are selected in the order listed.

63. The method of claim 56, wherein the cardiovascular disease is selected from at least one of: coronary heart disease (CHD), heart failure (HF), coronary heart disease (CHD), cerebrovascular disease, peripheral arterial disease, atherosclerosis, or heart failure.

64. The method of claim 56, wherein the method further comprises referring the subject to a specialist in cardiovascular disease.

65. The method of claim 38, claim 47, or claim 56, wherein step (b) is optionally omitted, and the immune cell subpopulations are measured directly from the sample.
